(19) United States
(12) Patent Application Publication
(10) Pub. No.: US 2007/0067004 A1
(43) Pub. Date: Mar. 22, 2007

(54) Methods and systems for modulating the vagus nerve (10th cranial nerve) to provide therapy for neurological, and neuropsychiatric disorders

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/602,776

(22) Filed: Nov. 21, 2006

Related U.S. Application Data
(63) Continuation-in-part of application No. 10/436,017, filed on May 11, 2003.
(63) Continuation-in-part of application No. 11/482,878, filed on Jul. 8, 2006.
Application No. 11/482,878 is a continuation-in-part of application No. 10/841,995, filed on May 8, 2004, now Pat. No. 7,076,307.
Application No. 10/841,995 is a continuation-in-part of application No. 10/195,961, filed on Jul. 16, 2002, now Pat. No. 7,062,330.
Application No. 10/195,961 is a continuation-in-part of application No. 10/142,298, filed on May 9, 2002.
Application No. 11/482,878 is a continuation-in-part of application No. 11/251,492, filed on Oct. 14, 2005.
Application No. 11/251,492 is a continuation-in-part of application No. 10/436,017, filed on May 11, 2003.

(51) Int. Cl. A61N 1/18

(52) U.S. Cl. 607/45

(57) ABSTRACT

Method and systems for neuromodulating vagus nerve(s) to provide therapy for neurological and neuropsychiatric disorders comprises implantable and external components. The pulsed electrical stimulation to vagus nerve(s) is used for disorders such as epilepsy, depression, anxiety disorders, neurogenic pain, compulsive eating disorders, obesity, dementia including Alzheimer's disease, and migraines. The pulsed electrical pulses to vagus nerve(s) may be provided using various forms of implanted pulse generators or various forms of implanted stimulus-receiver used with an external stimulator. The external components such as the programmer may communicate with the implanted pulse generator (IPG) utilizing magnetic inductive coupling or via wireless telemetry. Further, the external components such as the programmer or external stimulator may also comprise circuitry for networking with remote computers. The remote telemetry circuitry therefore allows for interrogation or programming of implanted device, from a remote location over a wide area network.

This Application is a Continuation-in-Part of application Ser. No. 10/436,017 filed May 11, 2003.

This Application is also a Continuation-in-Part of application Ser. No. 11/482,878 filed Jul. 08, 2006, which is a continuation-in-Part of application Ser. No. 10/841,995 which is a continuation-in-Part of application Ser. No. 10/195,961 filed Jul. 17, 2002, which is a continuation-in-Part of application Ser. No. 10/142,298 filed on May 9, 2002, and application Ser. No. 11/482,878 is also a CIP of application Ser. No. 11/251,492 filed on Oct. 14, 2005, which is a CIP of application Ser. No. 10/436,017 filed May 11, 2003. The above applications are incorporated herein in their entirety by reference, not inconsistent with this application, and priority is claimed from these applications.

### FIELD OF INVENTION

The present invention relates to neuromodulation, more specifically neuromodulation of vagus nerve with pulsed electrical stimulation, to provide therapy for neurological and neuropsychiatric disorders.

### BACKGROUND

The 10^{th} cranial nerve or the vagus nerve plays a role in mediating afferent information from visceral organs to the brain. The vagus nerve arises directly from the brain, but unlike the other cranial nerves extends well beyond the head. At its farthest extension it reaches the lower parts of the intestines. The vagus nerve provides an easily accessible, peripheral route to modulate central nervous system (CNS) function. Observations on the profound effect of electrical stimulation of the vagus nerve on central nervous system (CNS) activity extends back to the 1930's.

Experimental studies have indicated that afferent vagus nerve stimulation alters regional cerebral blood flow (rCBF) by increasing cerebral blood flow to certain areas of the brain, and decreasing cerebral blood flow to other areas of the brain. Although afferent vagus nerve stimulation has a very different mechanism of action, it reveals similarities in changes of rCBF to those associated with pharmacological treatment, in particular increase of rCBF to the middle frontal gyrus, and a reduction of rCBF in the limbic system and associated regions. Another important process that happens with afferent vagus nerve stimulation is an increase in release of neurochemicals namely serotonin, norepinephrine, and epinephrine. The effect of release of these chemicals is anti-depressant, as well as, anti-epileptogenic.

The present patent disclosure is primarily directed to methods and systems for electrical stimulation or neuromodulation of vagus nerve, for providing adjunct therapy for neurological and neuropsychiatric disorders such as depression, anxiety disorders, autism, epilepsy and involuntary movement disorders including Parkinson's disease, neurogenic/psychogenic pain, obsessive compulsive disorders, compulsive eating disorders, bulimia, obesity, dementia including Alzheimer's disease, and migraines.

In the human body there are two vagal nerves (VN), the right VN and the left VN. Each vagus nerve is encased in the carotid sheath along with the carotid artery and jugular vein. The innervation of the right and left vagus nerves is different. The innervation of the right vagus nerve is such that stimulating it results in profound bradycardia (slowing of the heart rate). The left vagus nerve has some innervation to the heart, but mostly innervates the visceral organs such as the gastrointestinal tract. It is known that stimulation of the left vagus nerve does not cause substantial slowing of the heart rate or cause any other significant deleterious side effects.

### Background of Neuromodulation

Neuromodulation is generally considered as the therapeutic alteration of activity in the central, peripheral or autonomic nervous systems, electrically or pharmacologically. Neuromodulation in this patent disclosure comprises stimulation, selective stimulation, blocking of nerve impulses, selective blocking of certain types of fibers, stimulation with selective blocking, and selective stimulation with selective blocking. One of the fundamental features of the nervous system is its ability to generate and conduct electrical impulses. Most nerves in the human body are composed of thousands of fibers of different sizes. This is shown schematically in FIG. 1. The different sizes of nerve fibers, which carry signals to and from the brain, are designated by groups A, B, and C. The vagus nerve, for example, may have approximately 100,000 fibers of the three different types, each carrying signals. Each axon or fiber of that nerve conducts only in one direction, in normal circumstances. In the vagus nerve sensory fibers outnumber parasympathetic fibers four to one.

In a cross section of peripheral nerve it is seen that the diameter of individual fibers vary substantially, as is also shown schematically in FIG. 2. The largest nerve fibers are approximately 20 μm in diameter and are heavily myelinated (i.e., have a myelin sheath, constituting a substance largely composed of fat), whereas the smallest nerve fibers are less than 1 μm in diameter and are unmyelinated.

The diameters of group A and group B fibers include the thickness of the myelin sheaths. Group A is further subdivided into alpha, beta, gamma, and delta fibers in decreasing order of size. There is some overlapping of the diameters of the A, B, and C groups because physiological properties, especially in the form of the action potential, are taken into consideration when defining the groups. The smallest fibers (group C) are unmyelinated and have the slowest conduction rate, whereas the myelinated fibers of group B and group A exhibit rates of conduction that progressively increase with diameter.

Nerve cells have membranes that are composed of lipids and proteins (shown schematically in FIGS. 3A and 3B), and have unique properties of excitability such that an adequate disturbance of the cell's resting potential can trigger a sudden change in the membrane conductance. Under resting conditions, the inside of the nerve cell is approximately −90 mV relative to the outside. The electrical signaling capabilities of neurons are based on ionic concentration gradients between the intracellular and extracellular compartments. The cell membrane is a complex of a bilayer of lipid molecules with an assortment of protein molecules embedded in it (FIG. 3A), separating these two compartments. Electrical balance is provided by concentration gradients which are maintained by a combination of selective permeability characteristics and active pumping mechanism.

The lipid component of the membrane is a double sheet of phospholipids, elongated molecules with polar groups at one end and the fatty acid chains at the other. The ions that carry the currents used for neuronal signaling are among these water-soluble substances, so the lipid bilayer is also an insulator, across which membrane potentials develop. In biophysical terms, the lipid bilayer is not permeable to ions. In electrical terms, it functions as a capacitor, able to store charges of opposite sign that are attracted to each other but unable to cross the membrane. Embedded in the lipid bilayer is a large assortment of proteins. These are proteins that regulate the passage of ions into or out of the cell. Certain membrane-spanning proteins allow selected ions to flow down electrical or concentration gradients or by pumping them across.

These membrane-spanning proteins consist of several subunits surrounding a central aqueous pore (shown in FIG. 3B). Ions whose size and charge “fit” the pore can diffuse through it, allowing these proteins to serve as ion channels. Hence, unlike the lipid bilayer, ion channels have an appreciable permeability (or conductance) to at least some ions. In electrical terms, they function as resistors, allowing a predicable amount of current flow in response to a voltage across them.

A nerve cell can be excited by increasing the electrical charge within the neuron, thus increasing the membrane potential inside the nerve with respect to the surrounding extracellular fluid. As shown in FIG. 4, stimuli 4 and 5 are subthreshold, and do not induce a response. Stimulus 6 exceeds a threshold value and induces an action potential (AP) which will be propagated. The threshold stimulus intensity is defined as that value at which the net inward current (which is largely determined by Sodium ions) is just greater than the net outward current (which is largely carried by Potassium ions), and is typically around −55 mV inside the nerve cell relative to the outside (critical firing threshold). If however, the threshold is not reached, the graded depolarization will not generate an action potential and the signal will not be propagated along the axon. This fundamental feature of the nervous system i.e., its ability to generate and conduct electrical impulses, can take the form of action potentials, which are defined as a single electrical impulse passing down an axon. This action potential (nerve impulse or spike) is an “all or nothing” phenomenon, that is to say once the threshold stimulus intensity is reached, an action potential will be generated.

FIG. 5A illustrates a segment of the surface of the membrane of an excitable cell. Metabolic activity maintains ionic gradients across the membrane, resulting in a high concentration of potassium (K⁺) ions inside the cell and a high concentration of sodium (Na⁺) ions in the extracellular environment. The net result of the ionic gradient is a transmembrane potential that is largely dependent on the K⁺ gradient. Typically in nerve cells, the resting membrane potential (RMP) is slightly less than 90 mV, with the outside being positive with respect to inside.

To stimulate an excitable cell, it is only necessary to reduce the transmembrane potential by a critical amount. When the membrane potential is reduced by an amount ΔV, reaching the critical or threshold potential (TP); Which is shown in FIG. 5B. When the threshold potential (TP) is reached, a regenerative process takes place: sodium ions enter the cell, potassium ions exit the cell, and the transmembrane potential falls to zero (depolarizes), reverses slightly, and then recovers or repolarizes to the resting membrane potential (RMP).

For a stimulus to be effective in producing an excitation, it must have an abrupt onset, be intense enough, and last long enough. These facts can be drawn together by considering the delivery of a suddenly rising cathodal constant-current stimulus of duration d to the cell membrane as shown in FIG. 5B.

Cell membranes can be reasonably well represented by a capacitance C, shunted by a resistance R as shown by a simplified electrical model in diagram 5C, and shown in a more realistic electrical model in FIG. 6, where neuronal process is divided into unit lengths, which is represented in an electrical equivalent circuit. Each unit length of the process is a circuit with its own membrane resistance (rₘ), membrane capacitance (cₘ), and axonal resistance (rₐ).

When the stimulation pulse is strong enough, an action potential will be generated and propagated. As shown in FIG. 7, the action potential is traveling from right to left. Immediately after the spike of the action potential there is a refractory period when the neuron is either unexcitable (absolute refractory period) or only activated to sub-maximal responses by supra-threshold stimuli (relative refractory period). The absolute refractory period occurs at the time of maximal Sodium channel inactivation while the relative refractory period occurs at a later time when most of the Na⁺ channels have returned to their resting state by the voltage activated K⁺ current. The refractory period has two important implications for action potential generation and conduction. First, action potentials can be conducted only in one direction, away from the site of its generation, and secondly, they can be generated only up to certain limiting frequencies.

A single electrical impulse passing down an axon is shown schematically in FIG. 8. The top portion of the figure (A) shows conduction over mylinated axon (fiber) and the bottom portion (B) shows conduction over nonmylinated axon (fiber). These electrical signals will travel along the nerve fibers.

The information in the nervous system is coded by frequency of firing rather than the size of the action potential. This is shown schematically in FIG. 9. The bottom portion of the figure shows a train of action potentials.

In terms of electrical conduction, myelinated fibers conduct faster, are typically larger, have very low stimulation thresholds, and exhibit a particular strength-duration curve or respond to a specific pulse width versus amplitude for stimulation, compared to unmyelinated fibers. The A and B fibers can be stimulated with relatively narrow pulse widths, from 50 to 200 microseconds (μs), for example. The A fiber conducts slightly faster than the B fiber and has a slightly lower threshold. The C fibers are very small, conduct electrical signals very slowly, and have high stimulation thresholds typically requiring a wider pulse width (300-1,000 μs) and a higher amplitude for activation. Because of their very slow conduction, C fibers would not be highly responsive to rapid stimulation. Selective stimulation of only A and B fibers is readily accomplished. The requirement of a larger and wider pulse to stimulate the C fibers, however, makes selective stimulation of only C fibers, to the exclusion of the A and B fibers, virtually unachievable inasmuch as the large signal will tend to activate the A and B fibers to some extent as well. Vagus nerve stimulation with selective blocking can also be performed using the methods and system described later in this patent application. The use of multiple electrodes, multiple channels of providing electrical pulses, and various blocking techniques are also described later.

As shown in FIG. 10A, when the distal part of a nerve is electrically stimulated, a compound action potential is recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories as shown in the Table one below,

TABLE 1

| | Conduction | Fiber | |
|---|---|---|---|
| Fiber | Velocity | Diameter | |
| Type | (m/sec) | (μm) | Myelination |
| A Fibers | | | |
| Alpha | 70-120 | 12-20 | Yes |
| Beta | 40-70 | 5-12 | Yes |
| Gamma | 10-50 | 3-6 | Yes |
| Delta | 6-30 | 2-5 | Yes |
| B Fibers | 5-15 | <3 | Yes |
| C Fibers | 0.5-2.0 | 0.4-1.2 | No |

FIG. 10B further clarifies the differences in action potential conduction velocities between the Aδ-fibers and the C-fibers. For many of the application of current patent application, it is the slow conduction C-fibers that are stimulated by the pulse generator.

The modulation of nerve in the periphery, as done by the body, in response to different types of pain is illustrated schematically in FIGS. 11 and 12. As shown schematically in FIG. 11, the electrical impulses in response to acute pain sensations are transmitted to brain through peripheral nerve and the spinal cord. The first-order peripheral neurons at the point of injury transmit a signal along A-type nerve fibers to the dorsal horns of the spinal cord. Here the second-order neurons take over, transfer the signal to the other side of the spinal cord, and pass it through the spinothalamic tracts to thalamus of the brain. As shown in FIG. 12, duller and more persistent pain travel by another-slower route using unmyelinated C-fibers. This route made up from a chain of interconnected neurons, which run up the spinal cord to connect with the brainstem, the thalamus and finally the cerebral cortex. The autonomic nervous system also senses pain and transmits signals to the brain using a similar route to that for dull pain.

Vagus nerve stimulation, as performed by the system and method of the current patent application, is a means of directly affecting central function. FIG. 13 shows cranial nerves have both afferent pathway 19 (inward conducting nerve fibers which convey impulses toward the brain) and efferent pathway 21 (outward conducting nerve fibers which convey impulses to an effector). Vagus nerve is composed of 80% afferent sensory fibers carrying information to the brain from the head, neck, thorax, and abdomen. The sensory afferent cell bodies of the vagus reside in the nodose ganglion and relay information to the nucleus tractus solitarius (NTS).

The vagus nerve is composed of somatic and visceral afferents and efferents. Usually, nerve stimulation activates signals in both directions (bi-directionally). It is possible however, through the use of special electrodes and waveforms, to selectively stimulate a nerve in one direction only (unidirectionally). The vast majority of vagus nerve fibers are C fibers, and a majority are visceral afferents having cell bodies lying in masses or ganglia in the skull.

In considering the anatomy, the vagus nerve spans from the brain stem all the way to the splenic flexure of the colon. Not only is the vagus the parasympathetic nerve to the thoracic and abdominal viscera, it also the largest visceral sensory (afferent) nerve. Sensory fibers outnumber parasympathetic fibers four to one. In the medulla, the vagal fibers are connected to the nucleus of the tractus solitarius (viceral sensory), and three other nuclei. The central projections terminate largely in the nucleus of the solitary tract, which sends fibers to various regions of the brain (e.g., the thalamus, hypothalamus and amygdala).

As shown in FIG. 14, the vagus nerve emerges from the medulla of the brain stem dorsal to the olive as eight to ten rootlets. These rootlets converge into a flat cord that exits the skull through the jugular foramen. Exiting the Jugular foramen, the vagus nerve enlarges into a second swelling, the inferior ganglion.

In the neck, the vagus lies in a groove between the internal jugular vein and the internal carotid artery. It descends vertically within the carotid sheath, giving off branches to the pharynx, larynx, and constrictor muscles. From the root of the neck downward, the vagus nerve takes a different path on each side of the body to reach the cardiac, pulmonary, and esophageal plexus (consisting of both sympathetic and parasympathetic axons). From the esophageal plexus, right and left gastric nerves arise to supply the abdominal viscera as far caudal as the splenic flexure.

In the body, the vagus nerve regulates viscera, swallowing, speech, and taste. It has sensory, motor, and parasympathetic components. Table two below outlines the innervation and function of these components.

TABLE 2
Vagus Nerve Components

| Component fibers | Structures innervated | Functions |
|---|---|---|
| SENSORY | Pharynx. larynx, | General sensation |
| | esophagus, external ear | |
| | Aortic bodies, aortic arch | Chemo- and baroreception |
| | Thoracic and abdominal | |
| | viscera | |
| MOTOR | Soft palate, pharynx, | Speech, swallowing |
| | larynx, upper esophagus | |
| PARA- | Thoracic and abdominal | Control of cardiovascular |
| SYMPATHETIC | viscera | system, respiratory and |
| | | gastrointestinal tracts |

On the Afferent side, visceral sensation is carried in the visceral sensory component of the vagus nerve(s). As shown in FIGS. 15A and 15B, visceral sensory fibers from plexus around the abdominal viscera converge and join with the right and left gastric nerves of the vagus. These nerves pass upward through the esophageal hiatus (opening) of the diaphragm to merge with the plexus of nerves around the esophagus. Sensory fibers from plexus around the heart and lungs also converge with the esophageal plexus and continue up through the thorax in the right and left vagus nerves. As shown in FIG. 15B, the central process of the nerve cell bodies in the inferior vagal ganglion enter the medulla and descend in the tractus solitarius to enter the caudal part of the nucleus of the tractus solitarius. From the nucleus, bilateral connections important in the reflex control of cardiovascular, respiratory, and gastrointestinal functions are made with several areas of the reticular formation and the hypothalamus.

The afferent fibers project primarily to the nucleus of the solitary tract. FIG. 16 depicts the relationship of the vagus nerve(s) 54 to the spinal cord 26, solitary tract neucleus 14, and the overall brain structure.

The vagal anatomical pathways of particular relevance to this patent disclosure is that the vagal afferents traverse the brainstem in the solitary tract, terminating with synapses located mainly in the nuclei of the dorsal medullary complex of the vagus. Most vagal afferents synapse in various structures of the medulla. Among these structures, the solitary tract nucleus (NTS) receives the greatest number of vagal afferent synapses, and each vagus nerve synapses bilaterally on the NTS. The vagal afferents carry information concerning visceral sensation, somatic sensation, and taste.

Shown in conjunction with FIG. 17A, each vagus nerve bifurcates within the medulla, to synapse bilaterally on the NTS. The NTS is a bilateral pair of small nuclei located in the dorsal medullary complex of the vagus. The NTS extends as a tube-like structure above and below this level within the medulla and caudal pons, as was also shown in FIGS. 14, and 15B. The white matter of the tractus solitarius lies in the center of this gray-matter tube, which consists of the multiple subnuclei of the NTS. In addition to dense innervation by the vagus nerves 54, the NTS also receives projections from a very wide range of peripheral and central sources. Also shown in conjunction with FIG. 17A, the NTS projects most densely to the parabrachial nucleus of the pons, with different portions of the NTS projecting specifically to different subnuclei of the parabrachial nucleus.

The NTS projects to a wide variety of structures within the posterior fossa, including all of the other nuclei of the dorsal medullary complex, the parabrachial nucleus and other pontine nuclei, and the vermis and inferior portions of the cerebellar hemispheres. The NTS has been likened to a small brain within the larger brain. The NTS receives a wide range of somatic and visceral sensory afferents, and receives a wide range of projections from other brain regions, performs extensive information processing internally, and produces motor and autonomic efferent outputs. The NTS has highly complex intrinsic excitatory and inhibitory connections among its interneurons.

The vagal nerve afferents have widespread projections to cerebral structures mostly using three or more synapses. The NTS projects to several structures within the cerebral hemispheres, including hypothalamic nuclei (the periventricular nucleus, lateral hypothalamic area, and other nuclei), thalamic nuclei (including the ventral posteromedial nucleus, paraventricular nucleus and other nuclei), the central nucleus of the amygdala, the bed of nucleus of the stria terminalis, and the nucleus accumbens. This is also depicted schematically in FIG. 17B. Through these projections, the NTS can directly influence activities of extrapyramidal motor systems, ascending visceral sensory pathways, and higher autonomic systems. Through its projections to the amygdala, the NTS gains access to amygdala-hippocampus-entrohinal cortex pathways of the limbic system.

The vagus-NTS-parabrachial pathways support additional higher cerebral influences of vagal afferents, as shown schematically in FIG. 17A. The parabrachial nucleus projects to several structures within the cerebral hemipheres, including the hypothalamus (particularly the lateral hypothalamic area), the thalamus (particularly intralaminar nuclei and the parvicellular portion of the ventral posteromedial nucleus), the amygdata (particularly the central nucleus of the amygdala, but also basolateral and other amygdalar nuclei), the anterior insula, and infralimbic cortes, lateral prefrontal cortex, and other cortical regions. The anterior insula constitutes the primary gustatory cortex. Higher-order projections of the anterior insula are particularly dense in inferior and inferolateral frontal cortex of the limbic system. The parabrachial nucleus functions as a major autonomic relay and processing site for autonomic and gustatory information.

The medial reticular formation of the medulla receives afferent projections from the vagus, other cranial nerves, anterolateral tracts of the spinal cord, the substantia nigra, fastigial and dentate nuclei of the cerebellum, the globus pallidus, and widespread areas of cerebral cortex.

Vagal afferents also have access to two special neuromodulatory systems for the brain and spinal cord, via bulbar noradrenergic and serotonergic projections. The locus coeruleus is a collection of dorsal pontine neurons that provide extremely widespread noradrenergic innervation of the entire cortex, diencephlon and many other brain structures. Most afferents to the locus coeruleus arise from two medullary nuclei, the nucleus paragigantocellularis and the nucleus prepositus hypoglossi. The NTS projects to the locus coeruleus through two major disynaptic pathways, one via the nucleus paragigantocellularis and the other via the nucleus prepositus hypoglossi.

Vagal-locus coeruleus and vagal-raphe interaction are potentially relevant to VNS mechanisms, since the locus coeruleus is the major source of norepinephrine, and the raphe is the major source of serotonin in most of the brain. Norepinephrine and serotonin exert anti-depressant and anti-seizure effects, in addition to modulating normal thalamic and cortical activities.

Vagal physiology is central to integration of the brain with the periphery in multiple activities of the autonomic and limbic systems, the thalamus, insular cortex, the amygdala, and frontal cortex interact extensively in acute and chronic stress reactions, anxiety, arousal, and reactivity.

The effects of vagus nerve stimulation on brain activation and regional cerebral blood flow have been studied using various imaging techniques. Magnetic resonance spectroscopy (MRS), functional magnetic resonance imaging (fMRI), positron emission tomography (PET), and single photon emission computed tomography (SPECT) permit non-invasive, regional brain mapping of blood flow, glucose metabolism, neurotransmitter concentrations, neurorecptor availability, and other functions. Among these techniques, mapping of regional cerebral blood flow (rCBF) with PET has been employed extensively to study VNS. Relative or absolute regional cerebral blood flow (rCBF) measurements can be made using fMRI, PET, or SPECT. Rapidly occurring changes in regional brain blood flow are considered to primarily reflect changes in trans-synaptic neurotransmission.

In one functional imaging study of acute VNS effects in humans which was reported where stimulation was applied to the vagus nerve during the stimulator-on PET acquisitions. The two groups differed only in the power of stimulation applied to the vagus nerve. Acute VNS induced bilateral rCBF increases in the thalami, hypothalami, and insular and inferior frontal regions, but induced bilateral rCBF decreases in the amygdalae, posterior hippocampi and cingulate gyri. It was concluded that left cervical VNS acutely alters synaptic activities in a widespread and bilateral distribution over brain structures that receive polysynaptic projections from the left vagus nerve.

In summary, the left cervical vagus nerve synapses bilaterally upon the nucleus of the tractus solitarius, the medullary reticular formation, and other medullary nuclei. The nucleus of the tractus solitarius projects densely upon the parabrachial nucleus of the pons, which itself projects heavily to multiple thalamic nuclei, the amygdala, the insula and other cerebral structures. The nucleus of the tractus solitarius projects monosynaptically to several cerebellar sites, monosyaptically to the raphe nuclei (which provide serotonergic innervation of virtually the entire neuraxis), and disynaptically to the locus coeruleus (which provides noradrenergic innervation of virtually the entire neuraxis).

Therapeutic VNS induces widespread bilateral subcortical and cortical alteration of synaptic activity in humans. These VNS-induced alteration in synaptic activity are consistent with known anatomical pathways of central vagal projection. Higher-power VNS causes larger volumes of alteration in cerebral synaptic activities, when comparing groups with high or low levels of VNS.

The vagal afferents have a high degree of access to the major sites of higher processing for the central autonomic network, the reticular activating system (RAS), and the limbic system. The RAS and limbic system are relevant to this disclosure and are as follows.

The limbic system is a group of structures located on the medial aspect of each cerebral hemisphere and diencephalon. Its cerebral structures encircle the upper part of the brain stem, as is shown in conjunction with FIGS. 17C and 17D, which are lateral views of the brain, showing some of the structures that constitute the limbic system. The limbic system include parts of the rhinencephalon (the septal nuclei, cingulate gyrus, parahippocampal gyrus, dentate gyrus, C-shaped hippocampus), and part of the amygdala. In the diencephalon, the main limbic structures are the hypothalamus and the anterior nucleus of the thalamus. The fornix and other fiber tracts link these limbic system regions together.

The limbic system is the emotional or affective (feeling) brain, and is therefore relevant to this disclosure. Two parts that are especially important in emotions are the amygdala and the anterior part of the cingulate gyrus. The amygdala recognizes angry or fearful facial expressions, assesses danger, and elicits the fear response. The cingulate gyrus plays a role in expressing out emotions through gestures and resolves mental conflicts when we are frustrated.

Extensive connections between the limbic system and lower and higher brain regions allow the system to integrate and respond to a wide variety of environmental stimuli. Most limbic system output is relayed through the hypothalamus, which is the neural clearinghouse for both autonomic (visceral) function and emotional response

The limbic system also interacts with the prefrontal lobes, so there is an intimate relationship between our feelings (mediated by the emotional brain) and our thoughts (mediated by the cognitive brain). Particular limbic structures, -the hippocampal structures and amygdala- also play an important role in converting new information into long-term memories.

The reticular formation extends the length of the brain stem, as depicted in FIG. 17E. A portion of this formation, the reticular activating system (RAS), maintains alert wakefulness of the cerebral cortex. Ascending arrows in FIG. 17E indicate input of sensory systems to the RAS, and then reticular output via thalamic relays to the cerebral cortex. Other reticular nuclei are involved in the coordination of muscle activity. Their output is indicated by the arrow descending the brain stem.

It has been shown that VNS acutely induces rCBF alteration at sites that receive vagal afferents and higher-order projections, including dorsal medulla, somatosensory cortex (contralateral to stimulation), thalamus and cerebellum bilaterally, and several limbic structures (including hippocampus and amygdala bilaterally). The projections of the nucleus of the solitary tract are summarized in FIG. 17B. Because of the widespread projections of the Nucleus of the Solitary Tract, neuromodulation of the vagal afferent nerve fibers produce alleviation of symptoms of the neurological and neuropsychiatric disorders covered in this patent application, such as epilepsy, depression, involuntary movement disorders including Parkinson's disease, anxiety disorders, neurogenic pain, psycogenic pain, obsessive compulsive disorders, migraines, obesity, dementia including Alzheimer's disease, and the like.

FIG. 17F shows the effects of vagus nerve stimulation on brain activation and cerebral-blood flow using functional magnetic resonance (fMRI) as published by Narayanan et al. in 2002. The curve represents the sum of all activated voxels over the entire brain that are imaged. More actual clinical studies have been done.

Further, it is known that serotonergic (5-HT) and noradrenergic (NE) systems are involved in the pathophysiology of depression and in the mechanisms of action of antidepressants. It has been shown that vagus nerve stimulation induces a large time-dependant increase in basal neuronal firing in the brainstem nuclei, for serotonin and norepinephrine: the dorsal raphe nucleus and locus coeruleus respectively. All classes of antidepressant treatments, including NREs, ECT, and NK1 antagonists, act at least in part, by increasing 5-HT neurotransmission, however NE probably also plays an important role in antidepressant effects, and NE is thought to be involved in the pathophysiology of depression. Long-term SSRIs increase 5-HT neurotransmission, while decreasing spontaneous NE activity. Conversely, NRIs are efficient antidepressant treatments and seem to affect 5-HT neurotransmission as do dual 5-HT and NE reuptake inhibitors. Vagus nerve stimulation is able to induce an increased firing activity of both serotonergic and noradrenergic neurons. Furthermore, the firing rates of both 5-HT and NE neurons increase as length of vagus nerve stimulation therapy increases. This mirrors the trend noticed in clinical VNS studies where mean HRSD scores tend to decrease further over time, indicating clinical improvement.

### RELATED ART

U.S. Pat. Nos. 4,702,254, 4,867,164 and 5,025,807 (Zabara) generally disclose animal research and experimentation related to epilepsy and the like. Applicant's method of neuromodulation is significantly different than that disclosed in Zabara '254, '164″ and '807 patents.

U.S. Pat. No. 5,299,569 (Wernicke et al.) is directed to the use of implantable pulse generator technology for treating and controlling neuropsychiatric disorders including schizophrenia, depression, and borderline personality disorder.

U.S. Pat. No. 6,205,359 B1 (Boveja) and U.S. Pat. No. 6,356,788 B2 (Boveja) are directed to adjunct therapy for neurological and neuropsychiatric disorders using an implanted lead-receiver and an external stimulator.

U.S. Pat. No. 5,807,397 (Barreras) is directed to an implantable stimulator with replenishable, high value capacitive power source.

U.S. Pat. No. 5,193,539 (Schulman, et al) is generally directed to an addressable, implantable microstimulator that is of size and shape which is capable of being implanted by expulsion through a hypodermic needle. In the Schulman patent, up to 256 microstimulators may be implanted within a muscle and they can be used to stimulate in any order as each one is addressable, thereby providing therapy for muscle paralysis.

U.S. Pat. No. 5,405,367 (Schulman, et al) is generally directed to the structure and method of manufacture of an implantable microstimulator.

U.S. Pat. No. 6,622,041 B2 (Terry, Jr. et al.) is directed to treatment of congestive heart failure and autonomic cardiovascular drive disorders using implantable neurostimulator.

### REFERENCES

1) Salinsky M C, Burchiel K J. Vagus nerve stimulation has no effect on awake EEG rhythms in humans. Epilepsia 1993; 34: 299-304.
2) Hammond E J, Uthman B M, Reid S A, et al. Electrophysiological studies of vagus nerve stimulation in humans, I: EEG effects. Epilepsia 1992; 33 1013-1020.

### Related Art Teachings and Applicant's Methodology

The related art teachings of Zabara and Wernicke in general relies on the fact, that in anesthetized animals stimulation of vagal nerve afferent fibers evokes detectable changes of the EEG in all of the regions, and that the nature and extent of these EEG changes depends on the stimulation parameters. They postulated (Wernicke et al. U.S. Pat. No. 5,269,303) that synchronization of the EEG may be produced when high frequency (>70 Hz) weak stimuli activate only the myelinated (A and B) nerve fibers, and that desynchronization of the EEG occurs when intensity of the stimulus is increased to a level that activates the unmyelinated (C) nerve fibers.

The applicant's methodology is different, and among other things is based on cumulative effects of providing electrical pulses to the vagus nerve(s) its branches or parts thereof. Complex electrical pulses are provided to vagus nerve(s) to cause changes to regional cerebral blood flow (rCBF) to selective parts/regions of the brain according to the specific nature of the disorder, and/or alter neurochemicals in the brain. Electrical pulses are provided to a patient without regard to synchronization or de-sychronization of patient's EEG. Further, in one aspect an open-loop system may be provided, where the physician determines the programs and/or parameters for stimulation and/or blocking for the patient.

The means and functionality of the applicant's disclosure does not rely on VNS-induced EEG changes, and is relevant since an intent of Zabara and Wernicke et al. teachings is to have a feedback system, wherein a sensor in the implantable system responds to EEG changes providing vagus nerve stimulation. In one aspect, Applicant's methodology is based on an open-loop system where the physician determines the parameters/programs for vagus nerve stimulation (and blocking). If the selected parameters or programs are uncomfortable, or are not tolerated by the patient, the electrical parameters are re-programmed. Advantageously, according to this disclosure, some re-programming or parameter adjustment may be done from a remote location, over a wide area network. A method of remote communication for neuromodulation therapy system is disclosed in commonly assigned U.S. Pat. No. 6,662,052 B1 and applicant's co-pending application Ser. No. 10/730,513 (Boveja), and are incorporated herein in their entirety by reference.

It is of interest that clinical investigation (in conscious humans) have not shown VNS-induced changes in the background EEGs of humans (References 1 and 2, by Salinsky M C and Hammond E J). A study, which used awake and freely moving animals, also showed no VNS-induced changes in background EEG activity. Taken together, the findings from animal study and human studies indicate that acute desynchronization of EEG activity is not a prominent feature of VNS when it is administered during physiologic wakefulness and sleep

One of the advantages of applicant's disclosure is that predetermined/prepackaged programs may be used. This may be done utilizing an inexpensive implantable pulse generator as disclosed in applicant's U.S. Pat. No. 6,760,626 B1 referred to as Boveja '626 patent. Predetermined/pre-packaged programs define program parameters such as pulse amplitude, pulse width, pulse frequency, on-time and off-time. Examples of predetermined/pre-packaged programs are disclosed in applicant's '626 patent, and in this disclosure for both implantable and external pulse generators. If an activated predetermined/pre-packaged program is uncomfortable for the patient, a different predetermined/pre-packaged program may be activated, or the program may be selectively modified.

Another advantage of applicant's methodology is that, at any given time a patient will receive the most aggressive therapy that is well tolerated. Since the therapy is cumulative the clinical benefits will be realized quicker.

Another advantage of the current disclosure is that complex electrical pulses may also be provided. Complex electrical pulses comprises at least one of multi-level pulses, biphasic pulses, rectangular pulses, non-rectangular pulses, or pulses with varying amplitude during the pulse. In some embodiments, complex pulses may also be used in conjunction with tripolar electrodes. The use of complex pulses adds another dimension to selective stimulation of vagus nerve, as recruitment of different fibers occurs during the pulse. The Zabara and Wernicke teachings utilize rectangular pulses.

After the patient has recovered from surgery (approximately 2 weeks), the stimulation/blocking is turned ON, and the effect of stimulation immediately is minimal. After a few weeks of intermittent stimulation, the effects start to become noticeable in some patients. Thereafter, the beneficial effects of pulsed electrical therapy accumulate up to a certain point, and are sustained over time, as the therapy is continued.

The method and systems of the present application may be similar to, or type of the method or system of the following documents. These documents describe various features and details associated with manufacture, operation, and use of the method and systems and are all incorporated herein by reference:

| Application | | |
|---|---|---|
| Serial No./ | | |
| Patent No: | Filing Date: | Title: |
| 11/035374 | Jan. 13, 2005 | Method and system for providing electrical pulses |
| | | for neuromodulation of vagus nerve(s), using |
| | | rechargeable implanted pulse generator. |
| 11/092124 | Mar. 29, 2005 | Method and system for providing therapy for autism by |
| | | providing electrical pulses to the vagus nerve(s). |
| 11/122645 | May 05, 2005 | Method and system for providing therapy for |
| | | Alzheimer's disease and dementia by providing |
| | | electrical pulses to vagus nerve(s). |
| 11/120125 | May 02, 2005 | Method and system for providing therapy for |
| | | bulimia/eating disorders by providing electrical |
| | | pulses to vagus nerve(s). |
| 11/223383 | Sep. 09, 2005 | Method and system to provide therapy or alleviate |
| | | symptoms of involuntary movement disorders by |
| | | providing complex and/or rectangular electrical |
| | | pulses to vagus nerve(s). |
| 11/126746 | May 10, 2005 | Method and system for providing therapy for |
| | | migraine/chronic headache by providing electrical |
| | | pulses to vagus nerve(s). |
| 11/126673 | May 11, 2005 | Method and system for providing adjunct (add-on) |
| | | therapy for depression, anxiety and obsessive- |
| | | compulsive disorders by providing electrical pulses |
| | | to vagus nerve(s). |
| 11/234,337 | Sep. 23, 2005 | System for providing electrical pulses to nerve |
| | | and/or muscle using an implanted stimulator. |
| 11/074130 | Mar. 07, 2005 | Method and system for providing therapy for |
| | | neuropsychiatric and neurological disorders utilizing |
| | | transcranical magnetic stimulation and pulsed |
| | | electrical vagus nerve(s) stimulation. |

### SUMMARY OF THE INVENTION

The method and systems of the current disclosure provides neuromodulation therapy using pulsed electrical stimulation to a cranial nerve such as a vagus nerve(s). The electrical stimulation is to provide therapy for at least one of depression, anxiety disorders, autism, epilepsy and involuntary movement disorders including Parkinson's disease, neurogenic/psychogenic pain, obsessive compulsive disorders, compulsive eating disorders, bulimia, obesity, dementia including Alzheimer's disease, and migraines.

The method and systems comprises both implantable and external components. The power source may also be external or implanted in the body. The system to provide electrical stimulation/blocking may be selected from a group comprising of:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator (IPG);

e) a combination implantable device comprising both a stimulus-receiver and a programmable IPG; and

f) an IPG comprising a rechargeable battery.

In one aspect of the disclosure, the electrical stimulation to a vagus nerve(s) may be anywhere along the length of the nerve, such as at the cervical level or at a level near the diaphram.

In another aspect of the disclosure, the stimulation may be unilateral or bilateral.

In another aspect of the disclosure, the external components such as the external stimulator or programmer comprise telemetry means adapted to be networked, for remote interrogation or remote programming of the device.

In another aspect of the disclosure, a programmable pulse generator may be implanted in the body.

In another aspect of the disclosure, predetermined/pre-packaged programs may be used.

In another aspect of the disclosure, one predetermined/pre-packaged programs is ON/OFF.

In another aspect of the disclosure, the predetermined/pre-packaged programs can be altered or modified.

In another aspect of the disclosure, a predetermined program may be customized for the patient.

In another aspect of the disclosure, a patient controller/programmer is provided.

In another aspect of the disclosure, a patient may adjust predetermined/pre-packaged program within predefined limits utilizing a patient controller/programmer.

In another aspect of the disclosure, the implanted pulse generator is adapted to be re-chargable via an external power source.

In another aspect of the disclosure, the predetermined/pre-packaged programs define unique combinations of variable electrical parameters.

In another aspect of the disclosure, the predetermined/pre-packaged programs may cause changes in regional cerebral blood flow (rCBF), and/or alter neurochemicals in the brain, and/or alter neural activity in the brain.

In another aspect of the disclosure, the complex electrical pulses provided are in a range between 0 Hz and 5,000 Hz.

In another aspect of the disclosure, the predetermined/pre-packaged programs provide therapy or alleviate symptoms of said disorders independently of synchronization or desynchronization of patient's EEG.

In another aspect of the disclosure, the predetermined/pre-packaged programs can be remotely interrogated and/or programmed using a network.

In another aspect of the disclosure, the implantable pulse generator communicates wirelessly with a wearable computer on a patient, and further the wearable computer is capable of being networked with remote computers.

In another aspect of the disclosure, a rechargeable implantable pulse generator comprises a recharge coil which may be inside or outside a titanium case of the implantable pulse generator.

In another aspect of the disclosure, the programmer comprises circuitry for remote communication over a wide area network, such as the internet.

In another aspect of the disclosure, patients implanted with the implantable pulse generator are provided with a smart card which comprises device and/or patient information, which can also be updated.

In another aspect of the disclosure, the implantable pulse generator comprises a radiofrequency identification tag (RFID) within a header of the implantable pulse generator.

In another aspect of the disclosure, a patient being implanted with the implantable pulse generator is also injected with a radiofrequency identification tag (RFID) into the body, which comprises device and/or patient information.

In another aspect of the disclosure, the electrical pulses are provided alone or as adjunct therapy with at least one of drug therapy, transcranial magnetic stimulation (rTMS) therapy, or electroconvulsive therapy (ECT), in any combination or sequence to provide therapy or alleviate symptoms of depression.

In another aspect of the disclosure, the implanted lead body may be made of a material selected from the group consisting of polyurethane, silicone, and silicone with polytetrafluoroethylene.

In another aspect of the disclosure, the implanted lead comprises at least one electrode selected from the group consisting of platinum, platinum/iridium alloy, platinum/iridium alloy coated with titanium nitride, and carbon.

In yet another aspect of the disclosure, the implanted lead comprises at least one electrode selected from the group consisting of spiral electrodes, cuff electrodes, steroid eluting electrodes, wrap-around electrodes, and hydrogel electrodes.

Various other features, objects and advantages of the disclosure will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram of the structure of a nerve.

FIG. 2 is a diagram showing different types of nerve fibers.

FIGS. 3A and 3B are schematic illustrations of the biochemical makeup of nerve cell membrane.

FIG. 4 is a figure demonstrating subthreshold and suprathreshold stimuli.

FIGS. 5A, 5B, 5C are schematic illustrations of the electrical properties of nerve cell membrane.

FIG. 6 is a schematic illustration of electrical circuit model of nerve cell membrane.

FIG. 7 is an illustration of propagation of action potential in nerve cell membrane.

FIG. 8 is an illustration showing propagation of action potential along a myelinated axon and non-myelinated axon.

FIG. 9 is an illustration showing a train of action potentials.

FIG. 10A is a diagram showing recordings of compound action potentials.

FIG. 10B is a schematic diagram showing conduction of first pain and second pain.

FIG. 11 is a schematic illustration showing mild stimulation being carried over the large diameter A-fibers.

FIG. 12 is a schematic illustration showing painful stimulation being carried over small diameter C-fibers

FIG. 13 is a schematic diagram of brain showing afferent and efferent pathways.

FIG. 14 is a schematic diagram showing the vagus nerve at the level of the nucleus of the solitary tract.

FIG. 15A is a schematic diagram showing the thoracic and visceral innervations of the vagal nerves.

FIG. 15B is a schematic diagram of the medullary section of the brain.

FIG. 16 is a schematic diagram of brain showing the relationship of the solitary tract nucleus to other centers of the brain.

FIG. 17A is a schematic diagram depicting connections of vagus nerve with solitary tract nucleus (NTS), parabrachial nucleus, and higher centers in the brain.

FIG. 17B is a simplified block diagram illustrating the connections of solitary tract nucleus to other centers of the brain.

FIGS. 17C and 17D are lateral view of the brain showing structures of the limbic system.

FIG. 17E is a diagram of the brain showing reticular activating system (RAS).

FIG. 17F is a graph showing activity curve on fMRI with periods of vagus nerve stimulation.

FIG. 17G depicts in table form, the peculiarities of different forms of device based therapies for neuropsychiatric disorders

FIG. 17H is a diagram depicting, where a patient receives repetitive Transcranial Magnetic Stimulation (rTMS) to the brain, and pulsed electrical stimulation to vagus nerve(s) with an implanted stimulator.

FIGS. 17-I and 17J show placement of ECT electrodes, where a patient receives electroconvulsive therapy (ECT), and pulsed electrical stimulation to vagus nerve(s) with an implanted stimulator.

FIG. 18 is a simplified block diagram depicting supplying amplitude and pulse width modulated electromagnetic pulses to an implanted coil.

FIG. 19 depicts a customized garment-for placing an external coil to be in close proximity to an implanted coil.

FIG. 20 is a diagram showing the implanted lead-receiver in contact with the vagus nerve at the distal end.

FIG. 21 is a schematic of the passive circuitry in the implanted lead-receiver.

FIG. 22A is a schematic of an alternative embodiment of the implanted lead-receiver.

FIG. 22B is another alternative embodiment of the implanted lead-receiver.

FIG. 23 shows coupling of the external stimulator and the implanted stimulus-receiver.

FIG. 24 is a top-level block diagram of the external stimulator and proximity sensing mechanism.

FIG. 25 is a diagram showing the proximity sensor circuitry.

FIG. 26A shows the pulse train to be transmitted to the vagus nerve.

FIG. 26B shows the ramp-up and ramp-down characteristic of the pulse train.

FIG. 27 is a schematic diagram of the implantable lead.

FIG. 28A is diagram depicting stimulating electrode-tissue interface.

FIG. 28B is diagram depicting an electrical model of the electrode-tissue interface.

FIG. 29 is a schematic diagram showing the implantable lead and one form of stimulus-receiver.

FIG. 30 is a schematic block diagram showing a system for neuromodulation of the vagus nerve, with an implanted component which is both RF coupled and contains a capacitor power source.

FIG. 31 is a simplified block diagram showing control of the implantable neurostimulator with a magnet.

FIG. 32 is a schematic diagram showing implementation of a multi-state converter.

FIG. 33 is a schematic diagram depicting digital circuitry for state machine.

FIG. 34 is a simplified block diagram of the implantable pulse generator.

FIG. 35A is a block diagram showing event detection sub-system and stimulation sub-system using dedicated leads and electrodes.

FIG. 35B is a block diagram showing event detection sub-system and stimulation sub-system using common leads and electrodes.

FIG. 36 is a functional block diagram of a microprocessor-based implantable pulse generator.

FIG. 37 shows details of implanted pulse generator.

FIGS. 38A and 38B shows details of digital components of the implantable circuitry.

FIG. 39A shows a schematic diagram of the register file, timers and ROM/RAM.

FIG. 39B shows datapath and control of custom-designed microprocessor based pulse generator.

FIG. 40 is a block diagram for generation of a pre-determined stimulation pulse.

FIG. 41 is a simplified schematic for delivering stimulation pulses.

FIG. 42 is a circuit diagram of a voltage doubler.

FIG. 43 shows a representative workable implantable pulse generator circuitry where a single chip microcontroller is used.

FIG. 44 is a block diagram of the Texas Instruments MSP430 microcontroller.

FIG. 45A shows amplifier and filtering circuitry connected to the analog inputs of the microcontroller.

FIG. 45B depicts a representative amplifier circuit.

FIG. 46A is a diagram depicting ramping-up of a pulse train.

FIG. 46B depicts rectangular pulses.

FIG. 46C depicts biphasic pulses.

FIGS. 46D, and 46E depict multi-step pulses.

FIGS. 46F, 46G, 46H, and 46-I depict complex pulse trains.

FIGS. 46J, 46K, 47L, 46M, 46N, and 46O are examples of complex pulses.

FIG. 47A depicts an implantable system with tripolar lead for selective unidirectional blocking of vagus nerve stimulation.

FIG. 47B depicts selective efferent blocking in the large diameter A and B fibers.

FIG. 48 is a schematic diagram of the implantable lead with three electrodes.

FIG. 49 is a diagram depicting electrical stimulation with conduction in the afferent direction and selective afferent block.

FIG. 50 is a diagram depicting electrical stimulation with conduction in the afferent direction and selective organ blocking in the efferent direction.

FIG. 51 is a diagram depicting electrical stimulation with conduction in the afferent and efferent direction and selective organ blocking in the efferent direction.

FIG. 52 depicts stimulation of vagus nerves at near the diaphragm level.

FIGS. 53A and 53B are diagrams showing communication of programmer with the implanted stimulator.

FIGS. 54A and 54B show diagrammatically encoding and decoding of programming pulses.

FIG. 55 is a simplified overall block diagram of implanted pulse generator (IPG) programmer.

FIG. 56 shows a programmer head positioning circuit.

FIG. 57 depicts typical encoding and modulation of programming messages.

FIG. 58 shows decoding one bit of the signal from FIG. 57.

FIG. 59 shows a diagram of receiving and decoding circuitry for programming data.

FIG. 60 shows a diagram of receiving and decoding circuitry for telemetry data.

FIG. 61 is a block diagram of a battery status test circuit.

FIG. 62A depicts communication of programmer with an implanted medical device utilizing magnetic inductive coupling.

FIG. 62B depicts communication of programmer with an implanted medical device utilizing wireless telemetry.

FIG. 63 shows the general components of a typical MICS RF system.

FIG. 64 depicts transceiver IC operating in the MICS band for vagal nerve modulation application.

FIGS. 65A and 65B show wireless telemetry antenna in the header region of implantable pulse generator.

FIG. 66 shows the power conservation features of a chip-based RF transceiver.

FIG. 67A depicts amplitude-shift keying.

FIG. 67B depicts on-off keying.

FIG. 68 is a block diagram of a low-power radio transceiver.

FIG. 69 shows detailed circuitry of the AMIS-52100 chip.

FIG. 70 is an example of a workable telemetry circuit.

FIG. 71 shows a block diagram of a Ultra-Low-Power MICS Transceiver architecture which uses frequency-shift-keyed (FSK) modulation with varying frequency deviations.

FIG. 72A depicts a patient holding a patent programmer over the implanted device.

FIG. 72B depicts one embodiment of a patient programmer with an optional external antenna attached.

FIGS. 73A and 73B depict front and back views of one embodiment of patient programmer with an antenna for remote communication over a wide area network.

FIGS. 74A and 74B show simplified block diagrams of two embodiments of patient programmer.

FIG. 75 is a diagram showing the two modules of the implanted pulse generator (IPG).

FIG. 76A depicts coil around the titanium case with two feedthroughs for a bipolar configuration.

FIG. 76B depicts coil around the titanium case with one feedthrough for a unipolar configuration.

FIG. 76C depicts two feedthroughs for the external coil which are common with the feedthroughs for the lead terminal.

FIG. 76D depicts one feedthrough for the external coil which is common to the feedthrough for the lead terminal.

FIG. 77 shows a block diagram of an implantable stimulator which can be used as a stimulus-receiver or an implanted pulse generator with rechargeable battery.

FIG. 78 is a block diagram highlighting battery charging circuit of the implantable stimulator.

FIG. 79 is a schematic diagram highlighting stimulus-receiver portion of implanted stimulator of one embodiment.

FIG. 80A depicts bipolar version of stimulus-receiver module.

FIG. 80B depicts unipolar version of stimulus-receiver module.

FIG. 81 depicts power source select circuit.

FIG. 82A shows energy density of different types of batteries.

FIG. 82B shows discharge curves for different types of batteries.

FIG. 83 depicts externalizing recharge and telemetry coil from the titanium case.

FIGS. 84A and 84B depict recharge coil on the titanium case with a magnetic shield in-between.

FIG. 85 shows in block diagram form an implantable rechargable pulse generator.

FIG. 86 depicts in block diagram form the implanted and external components of an implanted rechargable system.

FIG. 87 depicts the alignment function of rechargable implantable pulse generator.

FIG. 88 is a block diagram of the external recharger.

FIG. 89 depicts remote monitoring of stimulation devices.

FIG. 90 is an overall schematic diagram of the external stimulator, showing wireless communication.

FIG. 91 is a schematic diagram showing application of Wireless Application Protocol (WAP).

FIG. 92 is a simplified block diagram of the networking interface board.

FIGS. 93A and 93B is a simplified diagram showing communication of modified PDA/phone with an external stimulator via a cellular tower/base station.

FIG. 94 is a concept figure showing an implanted device communicating with a base station or repeater which is networked.

FIG. 95 is a block diagram of an exemplary base station or repeater.

FIG. 96 is an embodiment of networking using wireless telemetry.

FIG. 97 shows the signal flow between patient and a wearable computer.

FIG. 98 is a block diagram of a wearable computer.

FIGS. 99 and 100 show examples of wearable computers.

FIG. 101 is a block diagram of a memory card.

FIG. 102 is a block diagram showing typical architecture of a microprocessor card.

FIG. 103 shows an RFID tag placed in the header portion of an implanted pulse generator.

FIG. 104 shows an injectable RFID tag.

FIG. 105 shows the components of an RFID tag.

FIG. 106 shows an RFID reader (or interrogator) communicating with a transponder.

FIG. 107 shows an RFID tag encased in ceramic housing.

FIGS. 108 and 109 are diagrams showing the communication of RFID tag (transponder) with a reader.

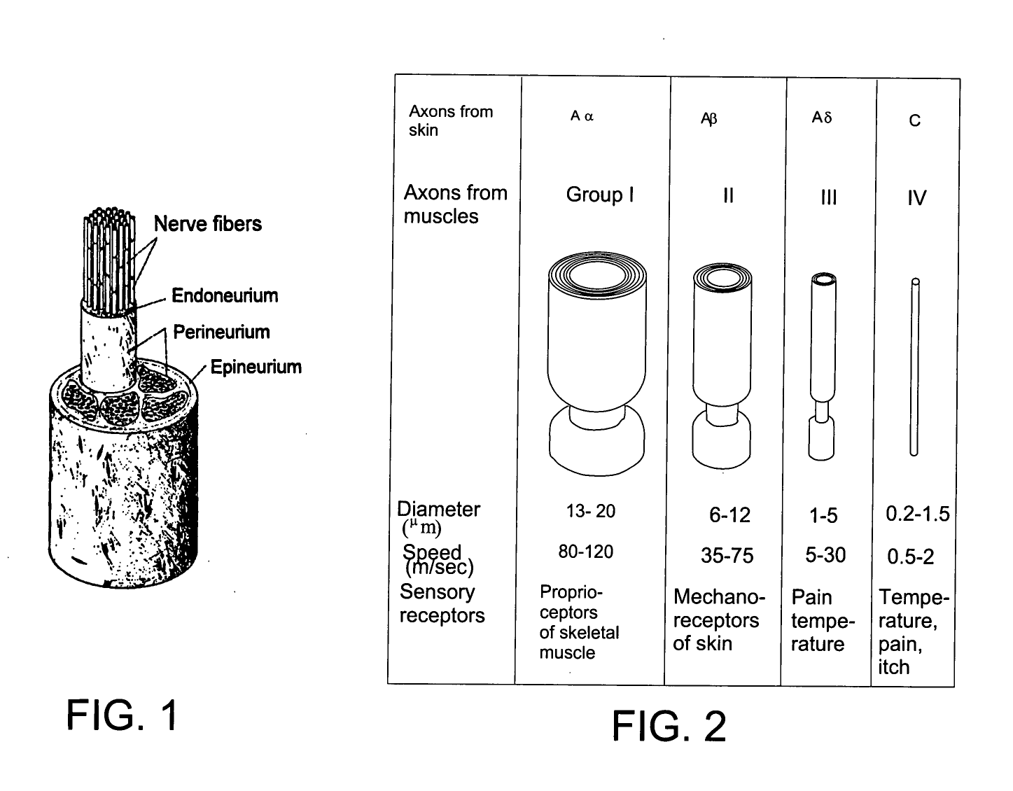

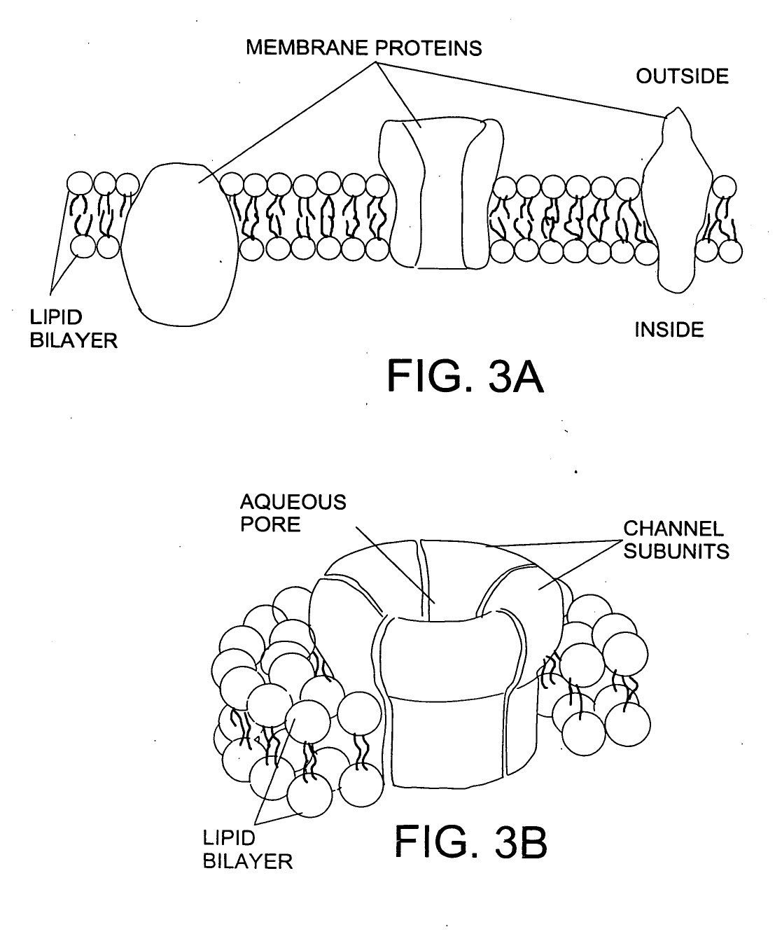

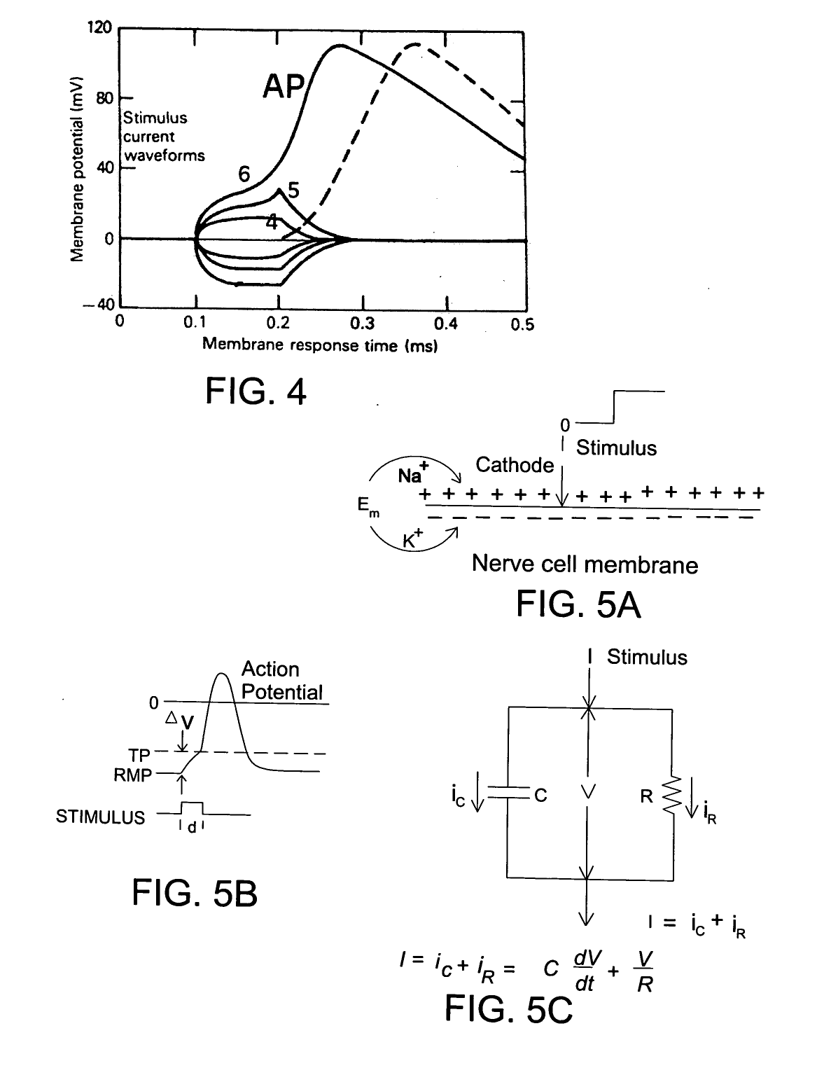

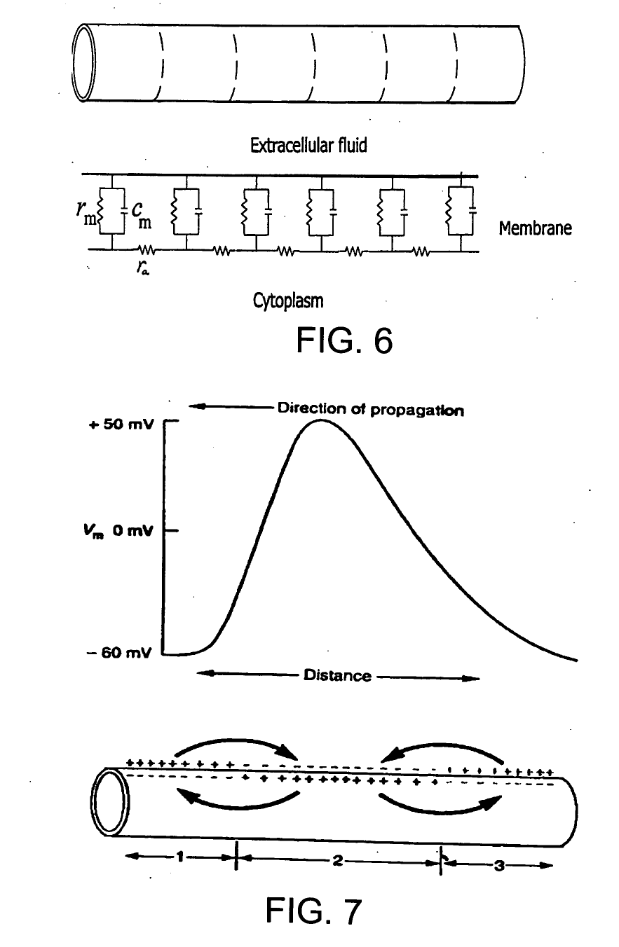

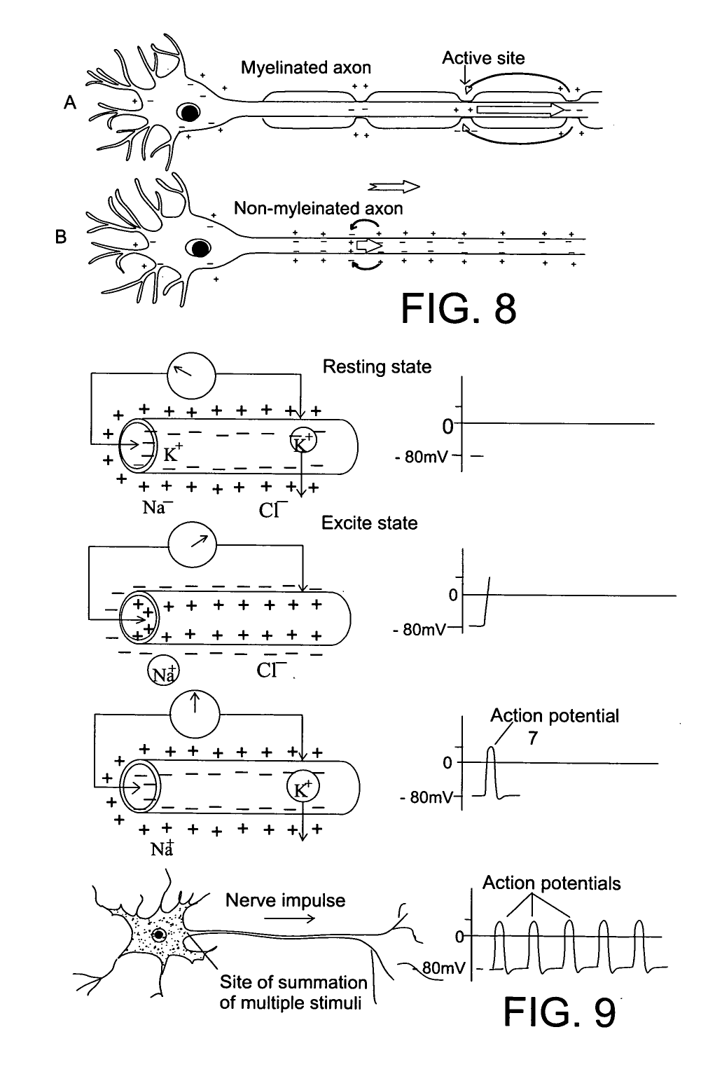

### DETAILED DESCRIPTION OF THE INVENTION

In the method and systems of this Application, electrical pulses are applied to a vagus nerve or branches or parts thereof for afferent neuromodulation. An implantable lead is surgically implanted in the patient. For some applications more than one lead may be implanted. The vagus nerve(s) is/are surgically exposed and isolated. The electrodes on the distal end of the lead are wrapped around the vagus nerve(s), and the lead is tunneled subcutaneously. A pulse generator means is connected to the proximal end of the lead. The power source may be external, implantable, or a combination device.

Additionally, in the method of this disclosure a cheaper and simpler pulse generator may be used to test a patient's response to neuromodulation therapy. As one example only, without limitation, an implanted stimulus-receiver in conjunction with an external stimulator may be used initially to test patient's response. At a later time, the pulse generator may be exchanged for a more elaborate implanted pulse generator (IPG) model, keeping the same lead. In general the physician determines which system would be most appropriate for each patient. Some examples of stimulation/blocking systems and power sources that may be used for the practice of this disclosure, and disclosed in this Application, include:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator (IPG);

e) a combination implantable device comprising both a stimulus-receiver and a programmable IPG; and

f) an IPG comprising a rechargeable battery.

In some applications, such as neuropsychiatric applications, particularly depression, vagus nerve stimulation and/or blocking may be provided as adjunct (add-on) therapy with other device based therapies such as, Transcranial Magnetic Stimulation (TMS) and/or Electroconvulsive therapy (ECT), in addition to pharmacological therapy.

### Afferent Vagus Nerve Stimulation (VNS) Used with Transcranial Magnetic Stimulation (rTMS)

In one aspect of the disclosure, afferent vagus nerve stimulation may be used with other pharmacological and non-pharmacological therapies. Drug therapy is typically the first line treatment for depression, and other central nervous system (CNS) disorders. Non-pharmacological treatments such as ECT and/or transcranial magnetic stimulation are particularly useful with afferent vagus nerve stimulation. Since ECT and transcranial magnetic stimulation (rTMS) approach the electrical or magnetic stimulation from outside the brain and vagus nerve stimulation approaches the brain from the inside. rTMS and ECT also work via different mechanism than vagus nerve stimulation. Applicant's co-pending application Ser. No. 11/074,130 entitled “Method and system for providing therapy for neuropsychiatric and neurological disorder utilizing transcranial magnetic stimulation and pulsed electrical vagus nerve(s) stimulation”, is incorporated herein by reference.

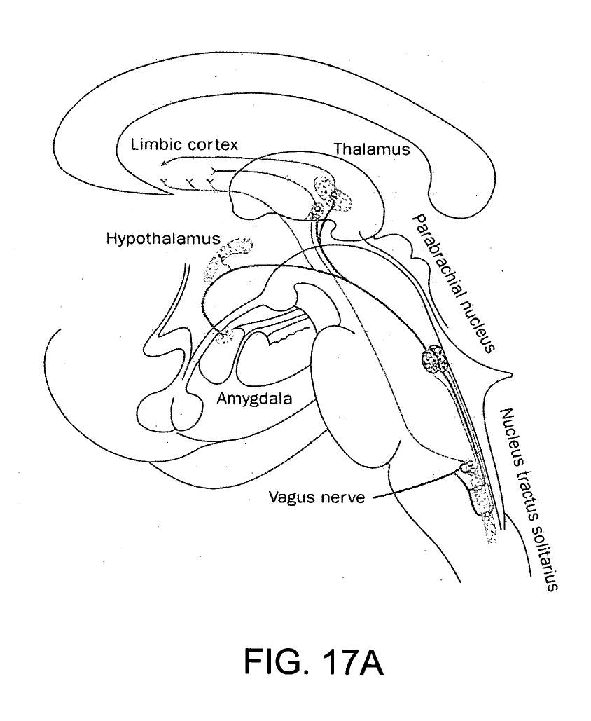

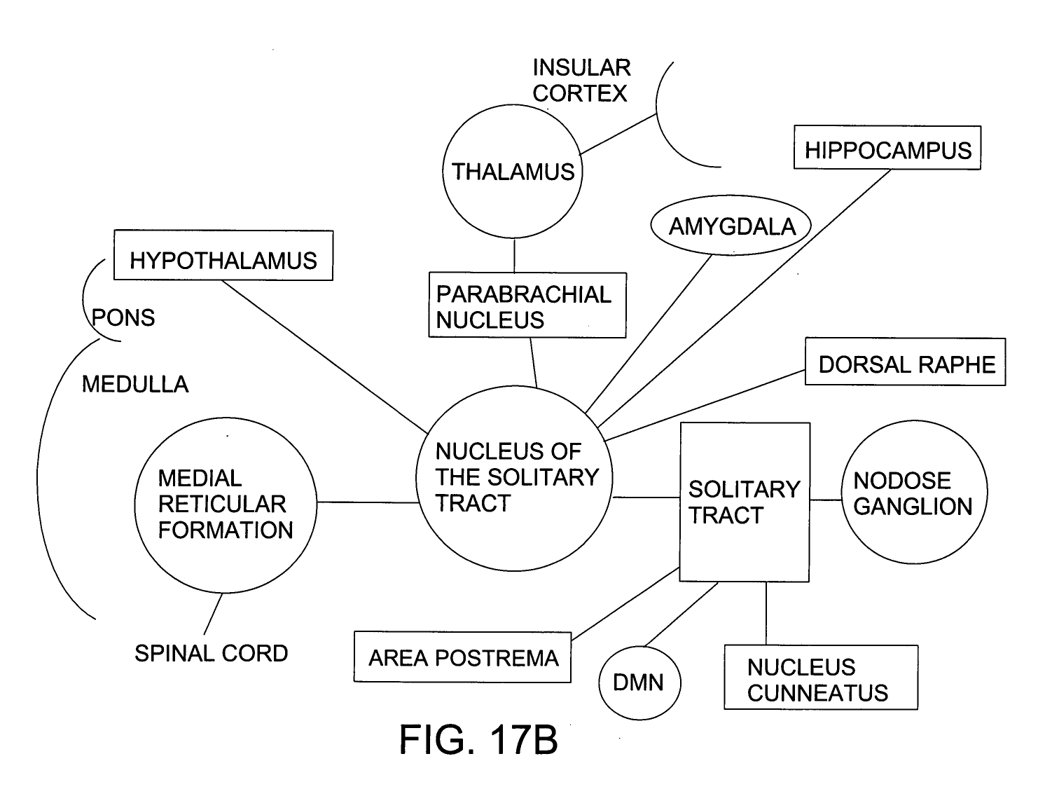

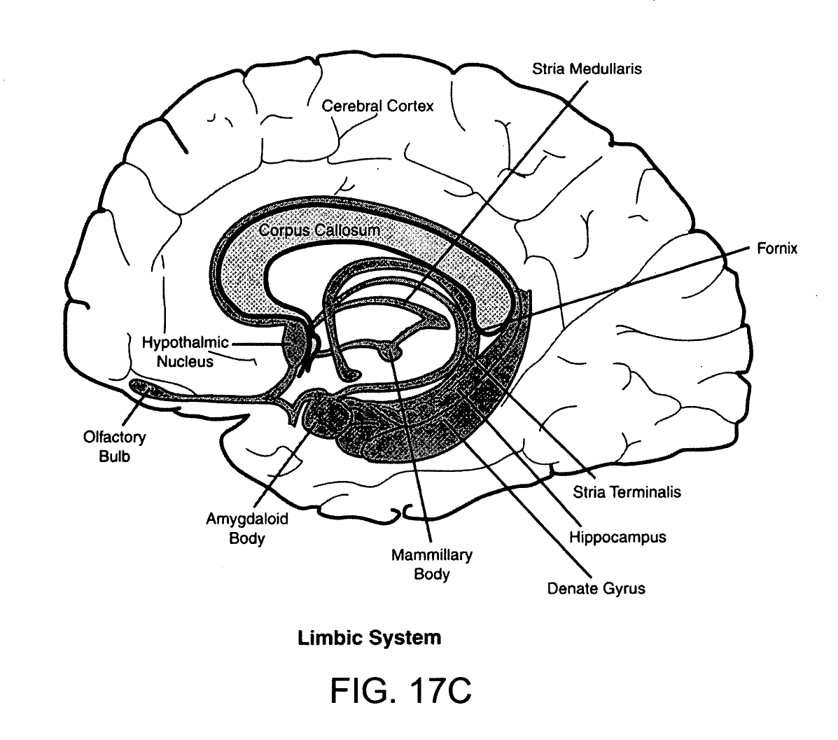

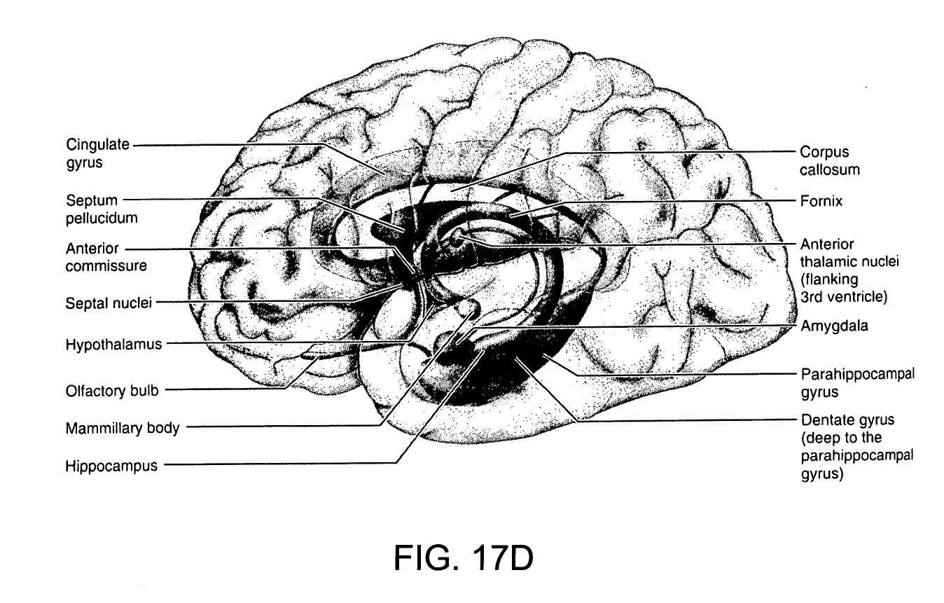

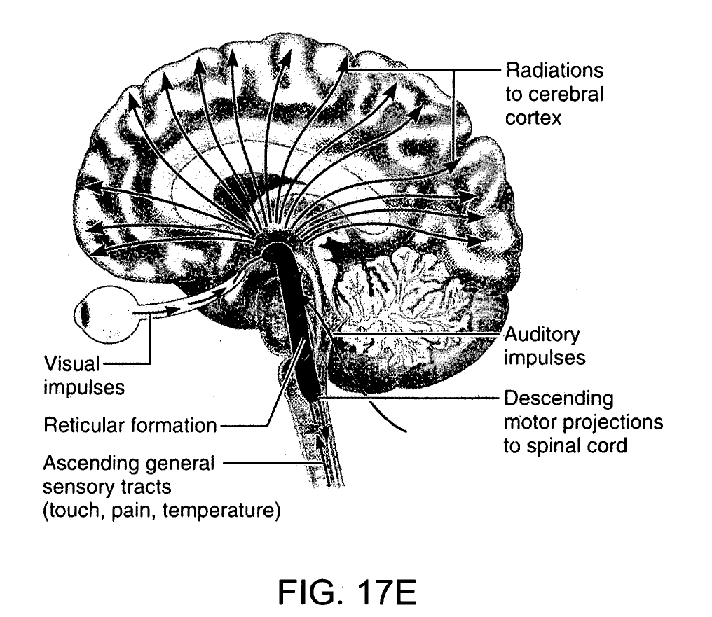

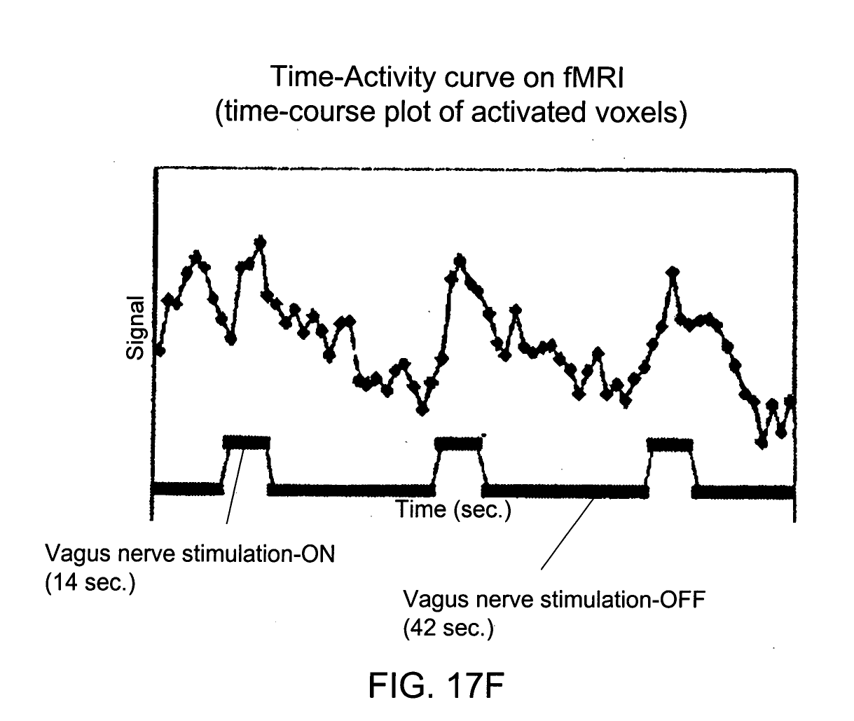

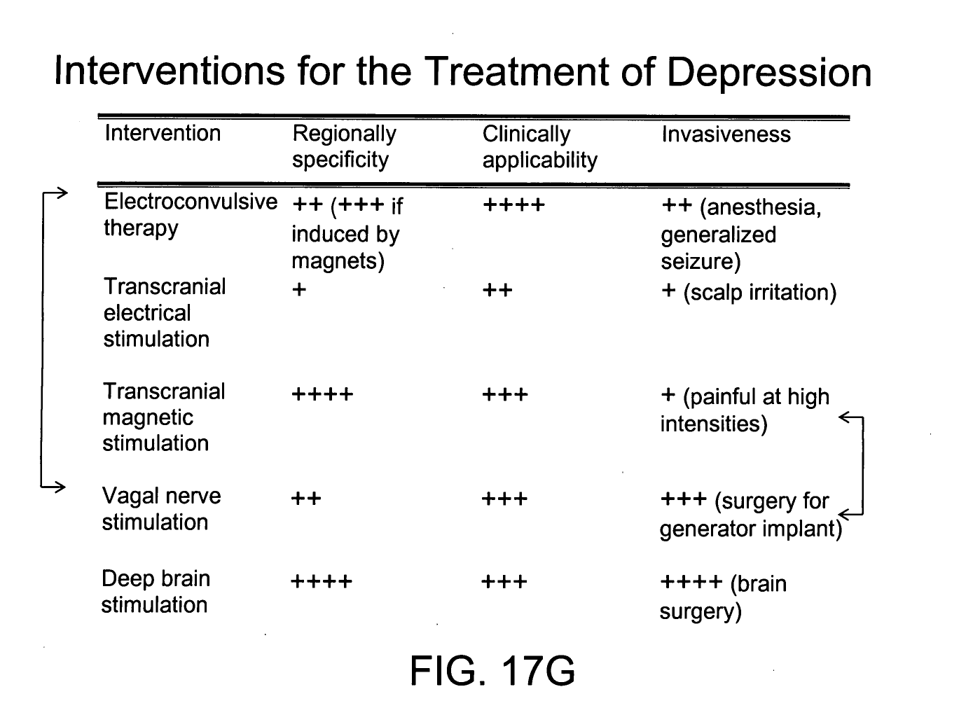

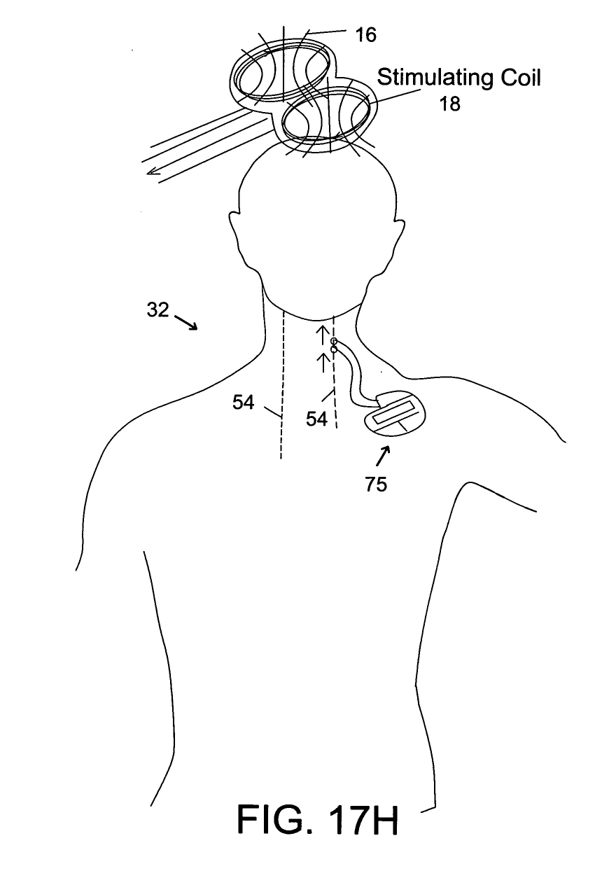

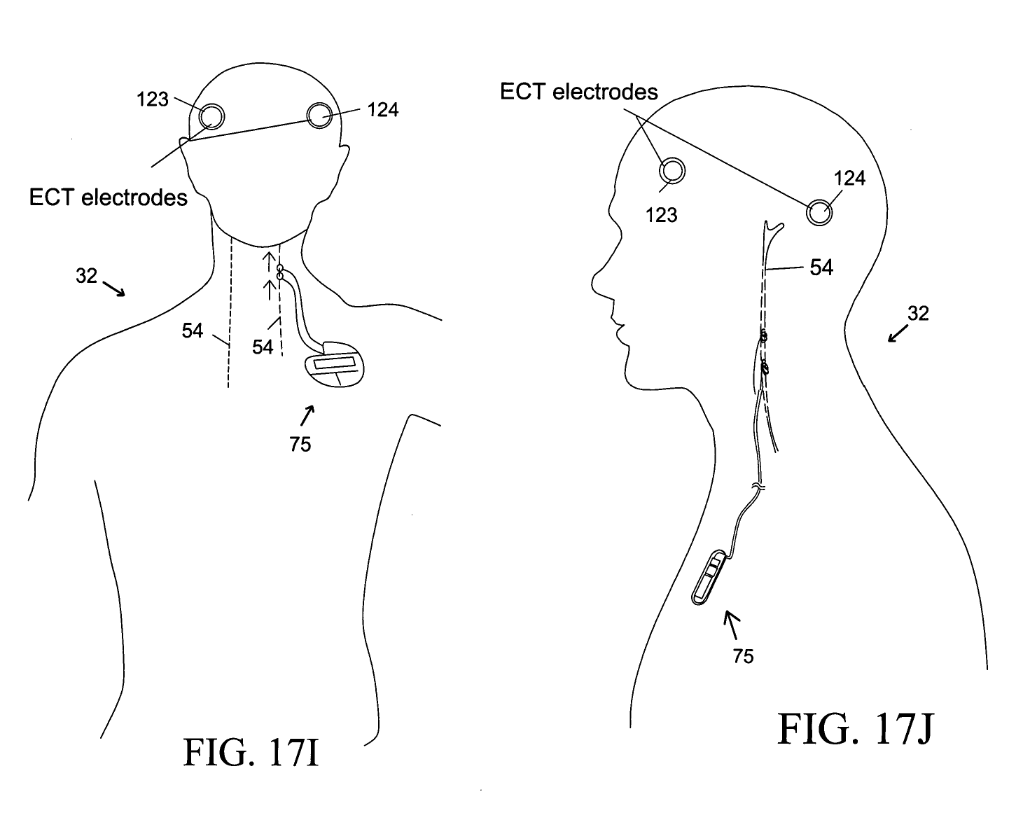

FIG. 17G (shown in table form) generally highlights some of the advantages and disadvantages of various forms of non-pharmacological interventions for the treatment of depression. Considering the advantages and disadvantages of different existing treatments, as shown in conjunction with FIG. 17G, a combination of rTMS therapy which involves changing magnetic fields and pulsed electrical vagus nerve stimulation is an ideal combination for device based interventions. The initiation and delivery of these two interventions may be in any sequence or combination, and may be in addition to any drug therapy, as determined by the physician. For example, a patient implanted with vagal nerve stimulator may be given rTMS therapy, or alternatively a patient receiving rTMS therapy may be implanted with a vagus nerve stimulator. Of course, this may be in addition to any drug therapy that may be given to a patient.

The combination use of rTMS and VNS is depicted in conjunction with FIG. 17H. In the method of this disclosure, the beneficial effects of rTMS and VNS would be synergistic or at least additive. The rationale for the combined systems is that with rTMS the electromagnetic energy is penetrated from outside to inside in changing magnetic fields, and with VNS the electrical pulses are delivered to the vagus nerve(s) 54, which provides stimulation (neuromodulation) from inside (i.e. from vagus nerve to brain stem to other projections in the brain). Further, the efficacy and invasiveness of the two stimulation therapies are also matched to provide the patient with balanced risk/benefit ratio. Electrical pulses to the vagus nerve(s) 54 are supplied using a pulse generator means and a lead with electrodes in contact with nerve tissue. rTMS are typically applied in short sessions. Vagus nerve stimulation is typically applied 24 hours/day, 7 days a week, in repeating cycles. The time periods of either rTMS or VNS may vary by any amount at the discretion of the physician.

Also shown in conjunction with Table-3 below, this combination balances the invasiveness, regional specificity and clinical applicability, and may be used with or without concomitant drug therapy. rTMS typically provides immediate benefits of mood improvement and no known side effects, but the benefits may or may not be very long lasting. With VNS the time profile of anti-depressant benefits are sustained over a long period of time, even though they may be slow to accumulate. Therefore, advantageously the combined benefits are both immediate and long lasting, providing a more ideal therapy profile, and cover a broader spectrum of patient population.

TABLE 3
Nonpharmacological interventions for the treatment of Depression and
other CNS disorders

| | Regionally | Clinically | |
|---|---|---|---|
| Intervention | specific | applicable | Invasive |
| Transcranial magnetic | ++++ | +++ | + (painful at high |
| stimulation | | | intensities) |
| Vagus nerve | ++ | +++ | +++ (surgery for |
| stimulation | | | generator implant) |

As mentioned previously, any combination, or sequence, or time intervals of these two energies may be applied, and is considered within the scope of the invention.

In some patients the beneficial effects of rTMS may last for sometime. These patient's may be implanted with the vagus nerve stimulator sometime after receiving their last dose of rTMS therapy. Typically patients who have received rTMS, and need a more aggressive therapy for treatment would be provided VNS. This form of combination therapy, where a patient receives rTMS therapy initially and sometime later receives pulsed electrical stimulation therapy, is also intended to be covered in the scope of this disclosure.

### ECT Used with Afferent Vagus Nerve Stimulation for Depression

Shown in conjunction with FIG. 17G were some advantages and disadvantages of various forms of nonpharmalogical interventions for the treatment of depression. As one example, ECT has clinical applicability in the short run, but on the other hand is associated with long-lasting cognitive impairments. Considering the advantages and disadvantages of different existing treatments, a combination of ECT therapy and pulsed electrical vagus nerve stimulation, as shown in conjunction with FIGS. 17-I and 17J is another desirable combination for device based interventions, with or without concomitant drug therapy. Furthermore, in this unique combination, ECT induces stimulation from outside, and vagus nerve stimulation (VNS) approaches the stimulation of centers in brain from inside. Interestingly, electroconvulsive therapy (ECT) is found to decrease prefrontal rCBF according to the majority of studies.

Based on this thinking as shown in conjunction with Table 4 below, which highlights that ECT and vagus nerve stimulation are an ideal combination of nonpharmalogical interventions, with or without concomitant drug therapy.

TABLE 4
Nonpharmacological interventions for the treatment of Depression

| | Regionally | Clinically | |
|---|---|---|---|
| Intervention | specific | applicable | Invasive |
| Electroconvulsive | ++ (+++ if | ++++ | ++ (anesthesia, |
| therapy (ECT) | induced by | | generalized seizure) |
| | magnets) | | |
| Vagus nerve | ++ | +++ | +++ (surgery for generator |
| stimulation | | | implant) |

The initiation and delivery of these two interventions may be in any sequence or combination, and may be in addition to any drug therapy. For example, a patient implanted with vagal nerve stimulator may be given ECT therapy, or alternatively a patient who has received ECT therapy previously may be implanted with a vagus nerve stimulator. Of course, this may be in addition to any drug therapy that may be given to a patient. It is an object of this disclosure to provide an optimal device based therapy for depression by supplementing ECT with VNS. ECT provided alone usually has cognitive adverse effects. Advantageously, not only would the cognitive adverse effects be reduced, but the efficacy would also be significantly improved by the combination of ECT and VNS as disclosed in this application.

Applicant's co-pending application Ser. No. 11/086,526, entitled “Method and system to provide therapy for depression using electroconvulsive therapy (ECT) and pulsed electrical stimulation to vagus nerve(s)” is incorporated herein by reference.

Several embodiments of pulse generator systems that may be used are described below.

### Implanted Stimulus-receiver with an External Stimulator

For an external power source, a passive implanted stimulus-receiver may be used. Such a system is disclosed in the parent application Ser. No. 10/142,298 and mentioned here for convenience.

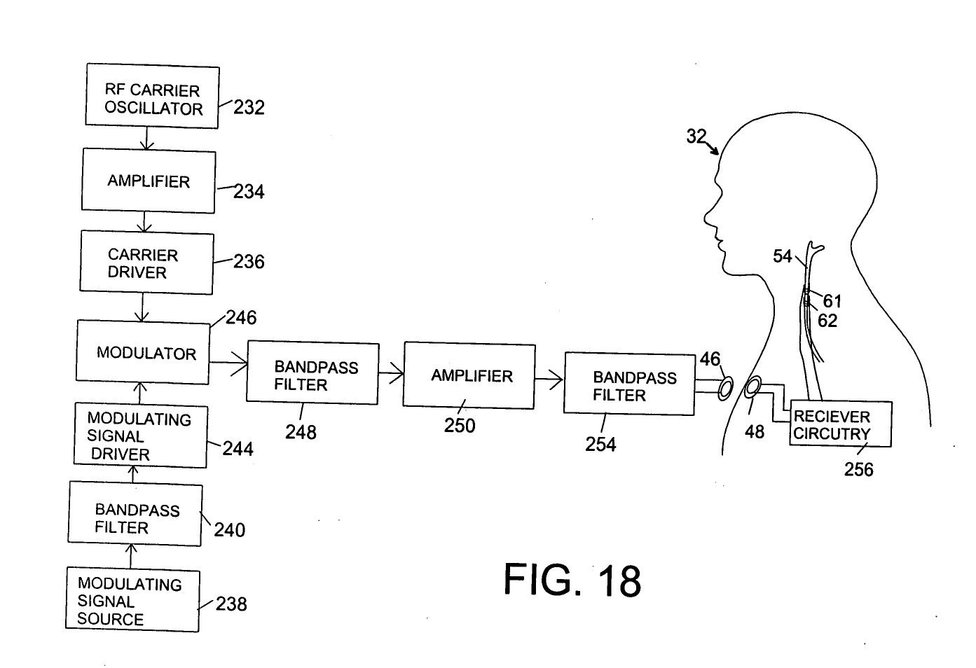

The selective stimulation of various nerve fibers of a cranial nerve such as the vagus nerve (or neuromodulation of the vagus nerve), as performed by one embodiment of the method and system of this invention is shown schematically in FIG. 18, as a block diagram. A modulator 246 receives analog (sine wave) high frequency “carrier” signal and modulating signal. The modulating signal can be multilevel digital, binary, or even an analog signal. In this embodiment, mostly multilevel digital type modulating signals are used. The modulated signal is amplified 250,252, conditioned 254, and transmitted via a primary coil 46 which is external to the body. A secondary coil 48 of an implanted stimulus receiver, receives, demodulates, and delivers these pulses to the vagus nerve 54 via electrodes 61 and 62. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

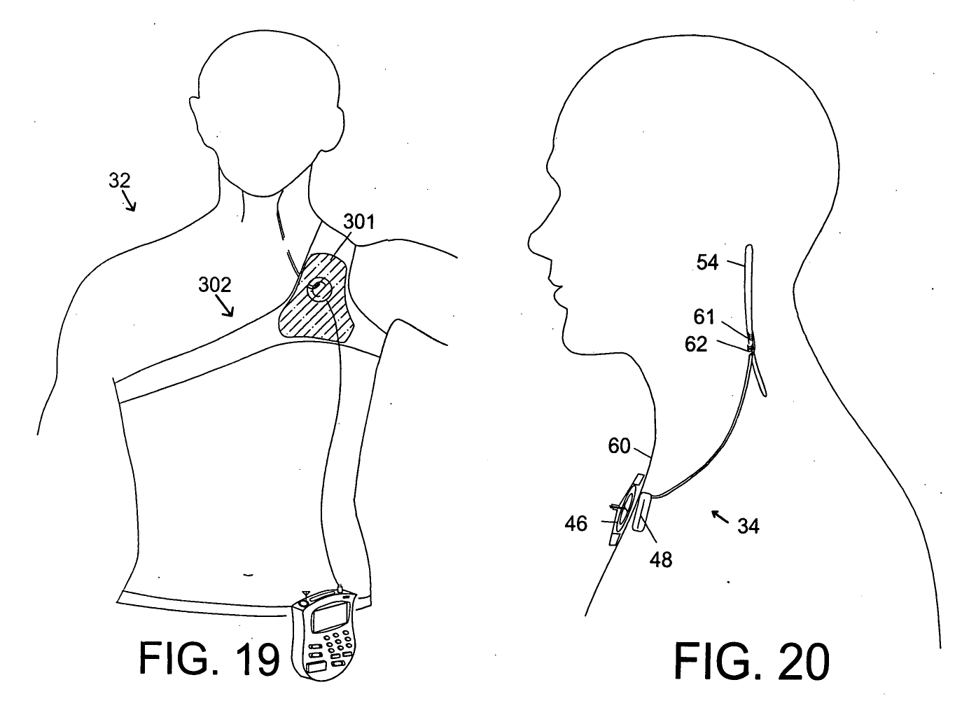

Shown in conjunction with FIG. 19, the coil for the external transmitter (primary coil 46) may be placed in the pocket 301 of a customized garment 302, for patient convenience.

Shown in conjunction with FIG. 20, the primary (external) coil 46 of the external stimulator 42 is inductively coupled to the secondary (implanted) coil 48 of the implanted stimulus-receiver 34. The implantable stimulus-receiver 34 has circuitry at the proximal end 49, and has two stimulating electrodes at the distal end 61,62. The negative electrode (cathode) 61 is positioned towards the brain and the positive electrode (anode) 62 is positioned away from the brain.

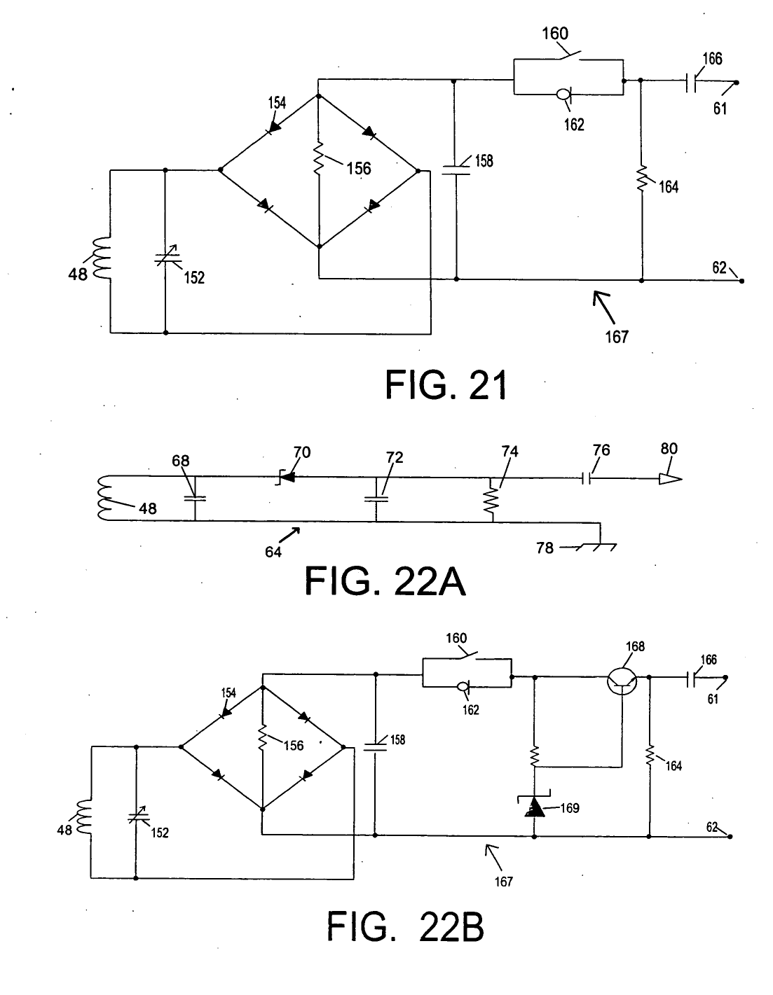

The circuitry contained in the proximal end of the implantable stimulus-receiver 34 is shown schematically in FIG. 21, for one embodiment. In this embodiment, the circuit uses all passive components. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, preferred in this embodiment. Unipolar stimulation is more likely to stimulate skeletal muscle in addition to nerve stimulation. The implanted circuit 167 in this embodiment is passive, so a battery does not have to be implanted.

The circuitry shown in FIGS. 22A and 22B can be used as an alternative, for the implanted stimulus-receiver. The circuitry of FIG. 22A is a slightly simpler version, and circuitry of FIG. 22B contains a conventional NPN transistor 168 connected in an emitter-follower configuration.

For therapy to commence, the primary (external) coil 46 is placed on the skin 60 on top of the surgically implanted (secondary) coil 48. An adhesive tape is then placed on the skin 60 and external coil 46 such that the external coil 46, is taped to the skin 60. For efficient energy transfer to occur, it is important that the primary (external) and secondary (internal) coils 46,48 be positioned along the same axis and be optimally positioned relative to each other. In this embodiment, the external coil 46 may be connected to proximity sensing circuitry 50. The correct positioning of the external coil 46 with respect to the internal coil 48 is indicated by turning “on” of a light emitting diode (LED) on the external stimulator 42.

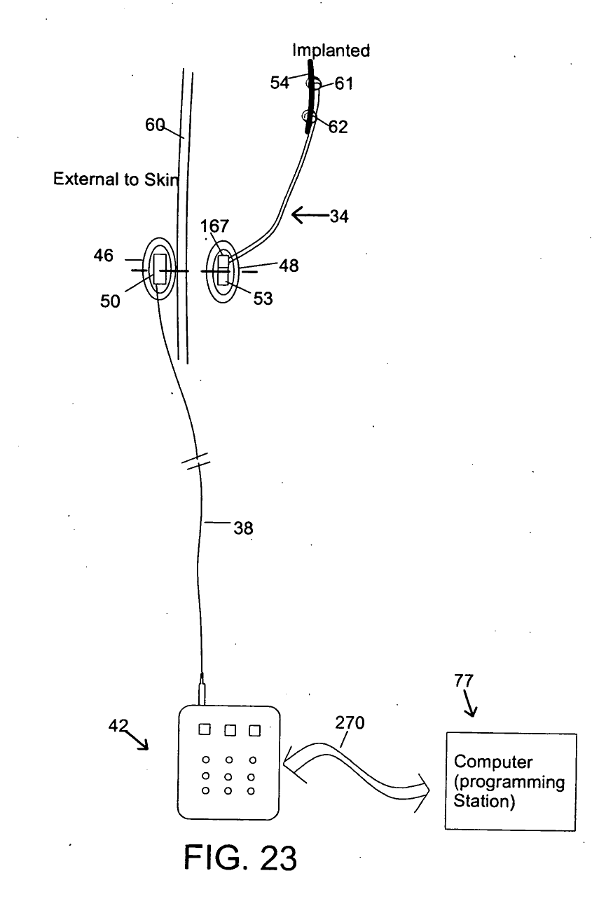

Optimal placement of the external (primary) coil 46 is done with the aid of proximity sensing circuitry incorporated in the system, in this embodiment. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. Shown in conjunction with FIG. 23, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42.

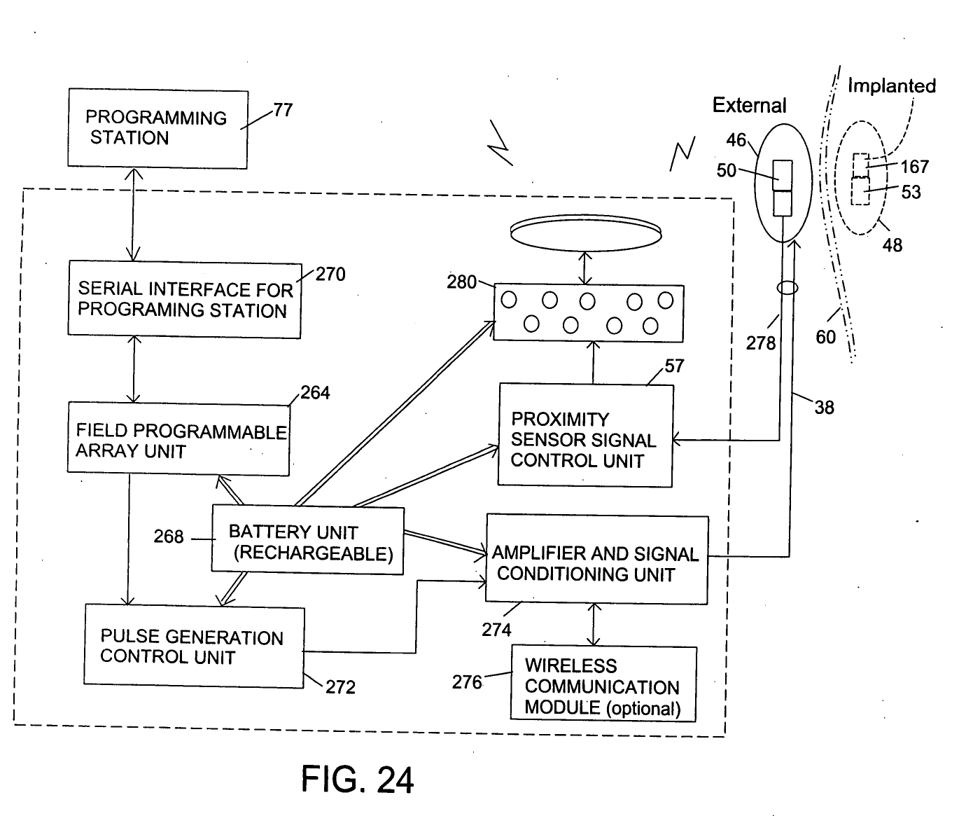

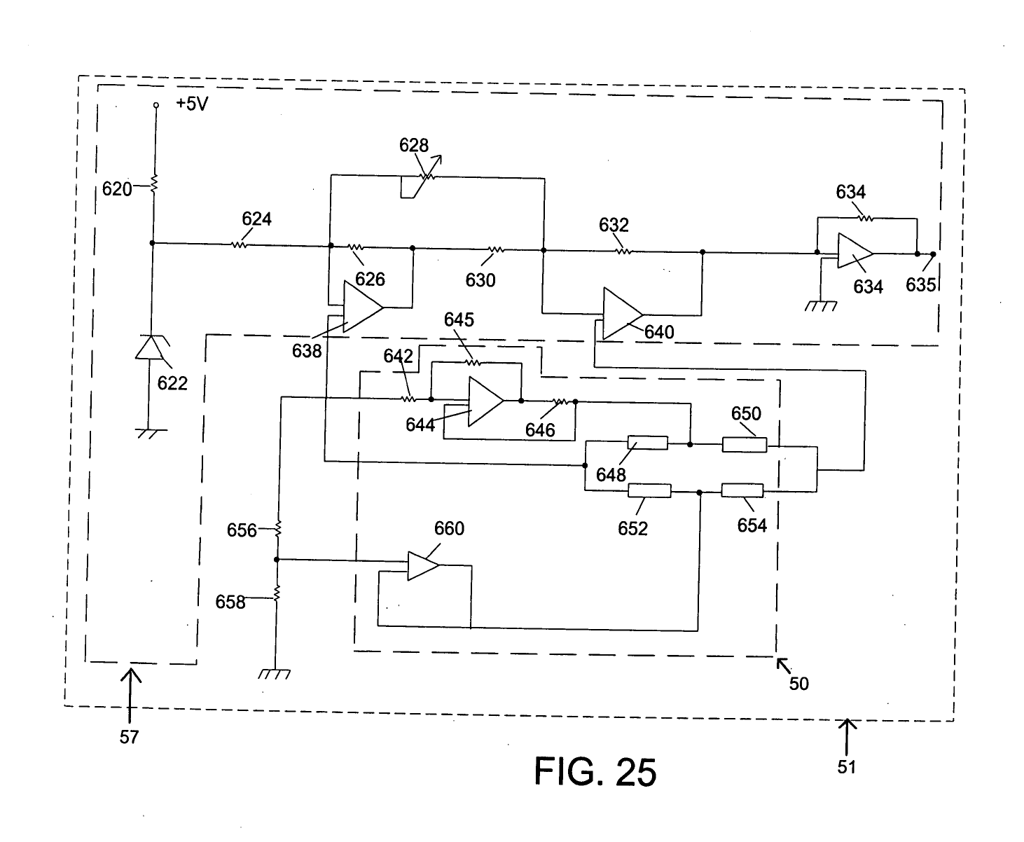

FIG. 24 shows an overall block diagram of the components of the external stimulator and the proximity sensing mechanism. The proximity sensing components are the primary (external) coil 46, supercutaneous (external) proximity sensors 648, 652 (FIG. 25) in the proximity sensor circuit unit 50, and a subcutaneous secondary coil 48 with a Giant Magneto Resister (GMR) magnet 53 associated with the proximity sensor unit. The proximity sensor circuit 50 provides a measure of the position of the secondary implanted coil 48. The signal output from proximity sensor circuit 50 is derived from the relative location of the primary and secondary coils 46, 48. The sub-assemblies consist of the coil and the associated electronic components, that are rigidly connected to the coil.

The proximity sensors (external) contained in the proximity sensor circuit 50 detect the presence of a GMR magnet 53, composed of Samarium Cobalt, that is rigidly attached to the implanted secondary coil 48. The proximity sensors, are mounted externally as a rigid assembly and sense the actual separation between the coils, also known as the proximity distance. In the event that the distance exceeds the system limit, the signal drops off and an alarm sounds to indicate failure of the production of adequate signal in the secondary implanted circuit 167, as applied in this embodiment of the device. This signal is provided to the location indicator LED 280.

FIG. 25 shows the circuit used to drive the proximity sensors 648, 652 of the proximity sensor circuit 50. The two proximity sensors 648, 652 obtain a proximity signal based on their position with respect to the implanted GMR magnet 53. This circuit also provides temperature compensation. The sensors 648, 652 are ‘Giant Magneto Resistor’ (GMR) type sensors packaged as proximity sensor unit 50. There are two components of the complete proximity sensor circuit. One component is mounted supercutaneously 50, and the other component, the proximity sensor signal control unit 57 is within the external stimulator 42. The resistance effect depends on the combination of the soft magnetic layer of magnet 53, where the change of direction of magnetization from external source can be large, and the hard magnetic layer, where the direction of magnetization remains unchanged. The resistance of this sensor 50 varies along a straight motion through the curvature of the magnetic field. A bridge differential voltage is suitably amplified and used as the proximity signal.

The Siemens GMR B6 (Siemens Corp., Special Components Inc., New Jersey) is used for this function in one embodiment. The maximum value of the peak-to-peak signal is observed as the external magnetic field becomes strong enough, at which point the resistance increases, resulting in the increase of the field-angle between the soft magnetic and hard magnetic material. The bridge voltage also increases. In this application, the two sensors 648, 652 are oriented orthogonal to each other.

The distance between the magnet 53 and sensor 50 is not relevant as long as the magnetic field is between 5 and 15 KA/m, and provides a range of distances between the sensors 648, 652 and the magnetic material 53. The GMR sensor registers the direction of the external magnetic field. A typical magnet to induce permanent magnetic field is approximately 15 by 8 by 5 mm³, for this application and these components. The sensors 648, 652 are sensitive to temperature, such that the corresponding resistance drops as temperature increases. This effect is quite minimal until about 100° C. A full bridge circuit is used for temperature compensation, as shown in temperature compensation circuit 50 of FIG. 25. The sensors 648, 652 and a pair of resistors 650, 654 are shown as part of the bridge network for temperature compensation. It is also possible to use a full bridge network of two additional sensors in place of the resistors 650, 654.

The signal from either proximity sensor 648, 652 is rectangular if the surface of the magnetic material is normal to the sensor and is radial to the axis of a circular GMR device. This indicates a shearing motion between the sensor and the magnetic device. When the sensor is parallel to the vertical axis of this device, there is a fall off of the relatively constant signal at about 25 mm. separation. The GMR sensor combination varies its resistance according to the direction of the external magnetic field, thereby providing an absolute angle sensor. The position of the GMR magnet can be registered at any angle from 0 to 360 degrees.

In the external stimulator 42 shown in FIG. 24, an indicator unit 280 which is provided to indicate proximity distance or coil proximity failure (for situations where the patch containing the external coil 46, has been removed, or is twisted abnormally etc.). Indication is also provided to assist in the placement of the patch. In case of general failure, a red light with audible signal is provided when the signal is not reaching the subcutaneous circuit. The indicator unit 280 also displays low battery status. The information on the low battery, normal and out of power conditions forewarns the user of the requirements of any corrective actions.

Also shown in FIG. 24, the programmable parameters are stored in a programmable logic 264. The predetermined programs stored in the external stimulator are capable of being modified through the use of a separate programming station 77. The Programmable Array Logic Unit 264 and interface unit 270 are interfaced to the programming station 77. The programming station 77 can be used to load new programs, change the existing predetermined programs or the program parameters for various stimulation programs. The programming station is connected to the programmable array unit 75 (comprising programmable array logic 304 and interface unit 270) with an RS232-C serial connection. The main purpose of the serial line interface is to provide an RS232-C standard interface.

This method enables any portable computer with a serial interface to communicate and program the parameters for storing the various programs. The serial communication interface receives the serial data, buffers this data and converts it to a 16 bit parallel data. The programmable array logic 264 component of programmable array unit receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit 264, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

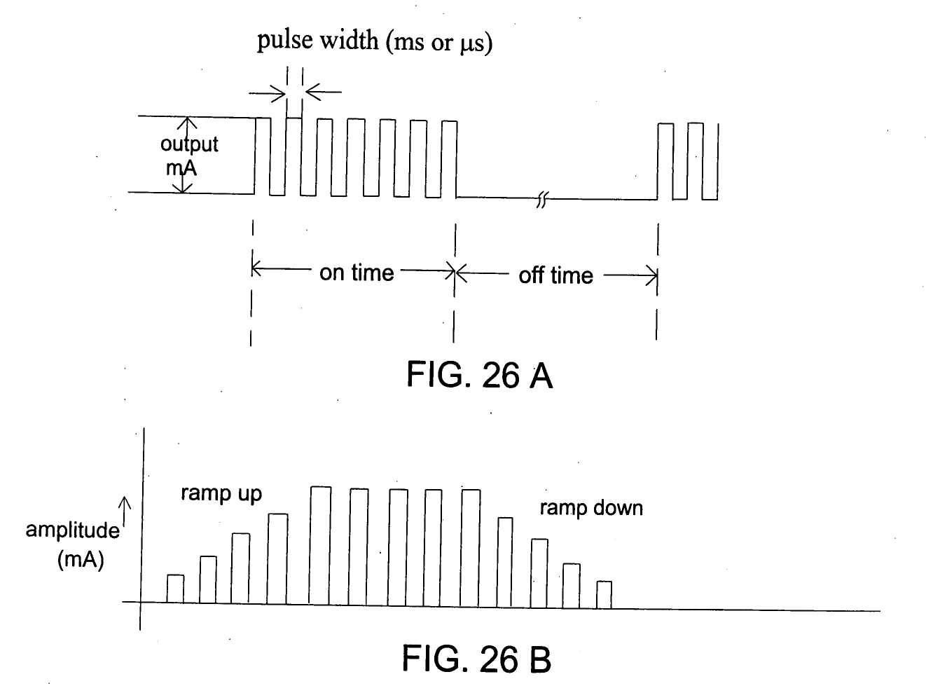

The pulses delivered to the nerve tissue for stimulation therapy are shown graphically in FIG. 26A. As shown in FIG. 26B, for patient comfort when the electrical stimulation is turned on, the electrical stimulation is ramped up and ramped down, instead of abrupt delivery of electrical pulses.

The electrical stimulation/blocking of the vagus nerve can be performed in one of two ways. One method is to activate one of several “predetermined” programs which are pre-packaged. A second method is to “custom” program the electrical parameters which can be selectively programmed, for specific therapy to the individual patient. The electrical parameters which can be individually programmed, include variables such as pulse amplitude, pulse width, frequency of stimulation, stimulation on-time, and stimulation off-time. Table five below defines the approximate range of parameters,

TABLE 5
Electrical parameter range delivered to the nerve

| PARAMER | RANGE |
|---|---|
| Pulse Amplitude | 0.1 Volt-20 Volts |
| Pulse width | 20 μS-5 mSec. |
| Frequency | 5 Hz-5,000 Hz |
| On-time | 5 Secs-24 hours |
| Off-time | 5 Secs-24 hours |

The parameters in Table 5 are the electrical signals delivered to the nerve via the two electrodes 61,62 (distal and proximal) around the nerve, as shown in FIG. 20. It being understood that the signals generated by the external pulse generator 42 and transmitted via the primary coil 46 are larger, because the attenuation factor between the primary coil and secondary coil is approximately 10-20 times, depending upon the distance, and orientation between the two coils. Accordingly, the range of transmitted signals of the external pulse generator are approximately 10-20 times larger than shown in Table 2.

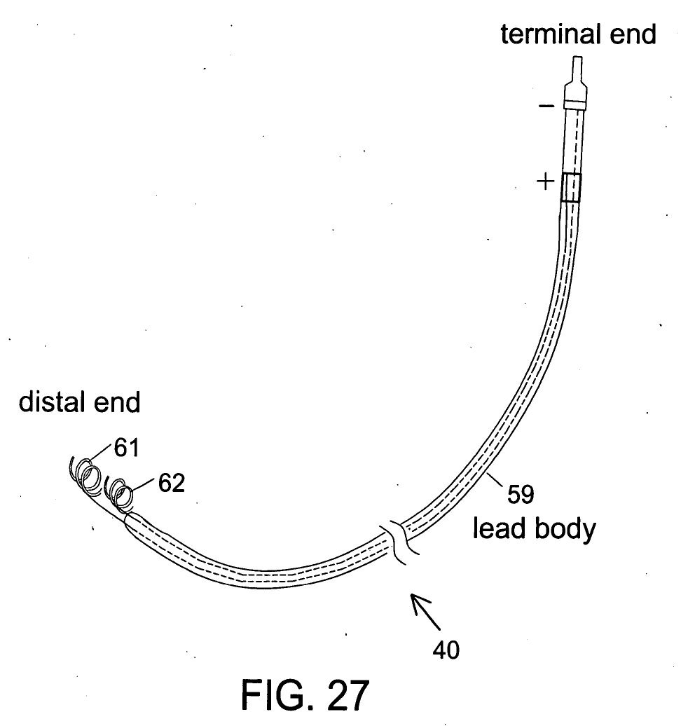

Referring now to FIG. 27, the implanted lead component of the system is similar to cardiac pacemaker leads, except for distal portion (or electrode end) of the lead. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62 for stimulating the vagus nerve 54 may either wrap around the nerve once or may be spiral shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table six below.

TABLE 6
Lead design variables

| | |
|---|---|
| Proximal | Distal |
| End | End |

| | | | Conductor | | |
|---|---|---|---|---|---|
| | | | (connecting | | |
| | Lead body- | | proximal | | |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Spiral |
| bipolar | | coating | Nickel- | Platinum | electrode |
| | | | Cobalt | | |
| Bifurcated | Silicone | Anti- | | Platinum- | Wrap-around |
| | | Inflammatory | | Iridium | electrode |
| | | coating | | (Pt/lr) Alloy | |
| | Silicone with | Lubricious | | Pt/lr coated | Steroid |
| | Polytetrafluoro- | coating | | with Titanium | eluting |
| | ethylene | | | Nitride | |
| | (PTFE) | | | | |
| | | | | Carbon | Hydrogel |
| | | | | | electrodes |
| | | | | | Cuff |
| | | | | | electrodes |

Once the lead is fabricated, coating such as anti-microbial, anti-inflammatory, or lubricious coating may be applied to the body of the lead.

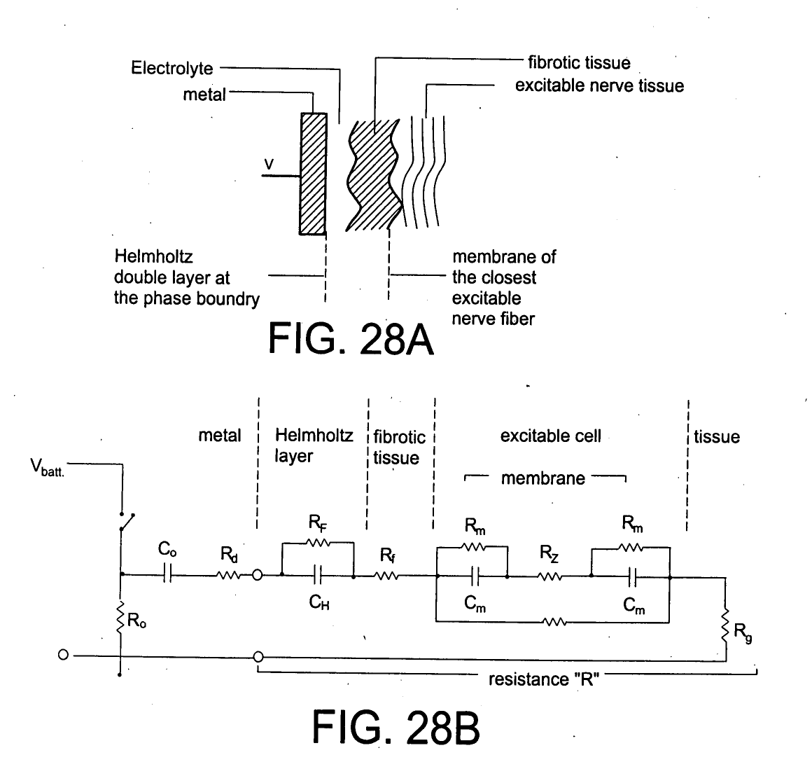

FIG. 28A summarizes electrode-tissue interface between the nerve tissue and electrodes 61, 62. There is a thin layer of fibrotic tissue between the stimulating electrode 61 and the excitable nerve fibers of the vagus nerve 54. FIG. 28B summarizes the most important properties of the metal/tissue phase boundary in an equivalent circuit diagram. Both the membrane of the nerve fibers and the electrode surface are represented by parallel capacitance and resistance. Application of a constant battery voltage Vbat from the pulse generator, produces voltage changes and current flow, the time course of which is crucially determined by the capacitive components in the equivalent circuit diagram. During the pulse, the capacitors Co, Ch and Cm are charged through the ohmic resistances, and when the voltage Vbat is turned off, the capacitors discharge with current flow on the opposite direction.

### Implanted Stimulus-receiver Comprising a High Value Capacitor for Storing Charge, Used in Conjunction with an External Stimulator

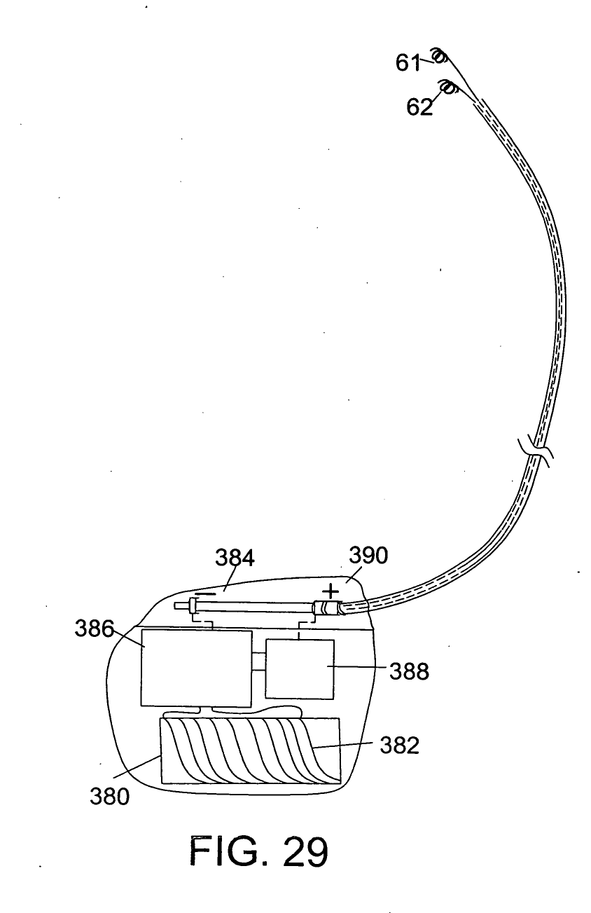

In one embodiment, the implanted stimulus-receiver may be a system which is RF coupled combined with a power source. In this embodiment (shown in FIG. 29), the implanted stimulus-receiver contains high value, small sized capacitor(s) for storing charge and delivering electric stimulation pulses for up to several hours by itself, once the capacitors are charged. Using mostly hybrid components and appropriate packaging, the implanted portion of the system described below is conducive to miniaturization. As shown in FIG. 29, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment.

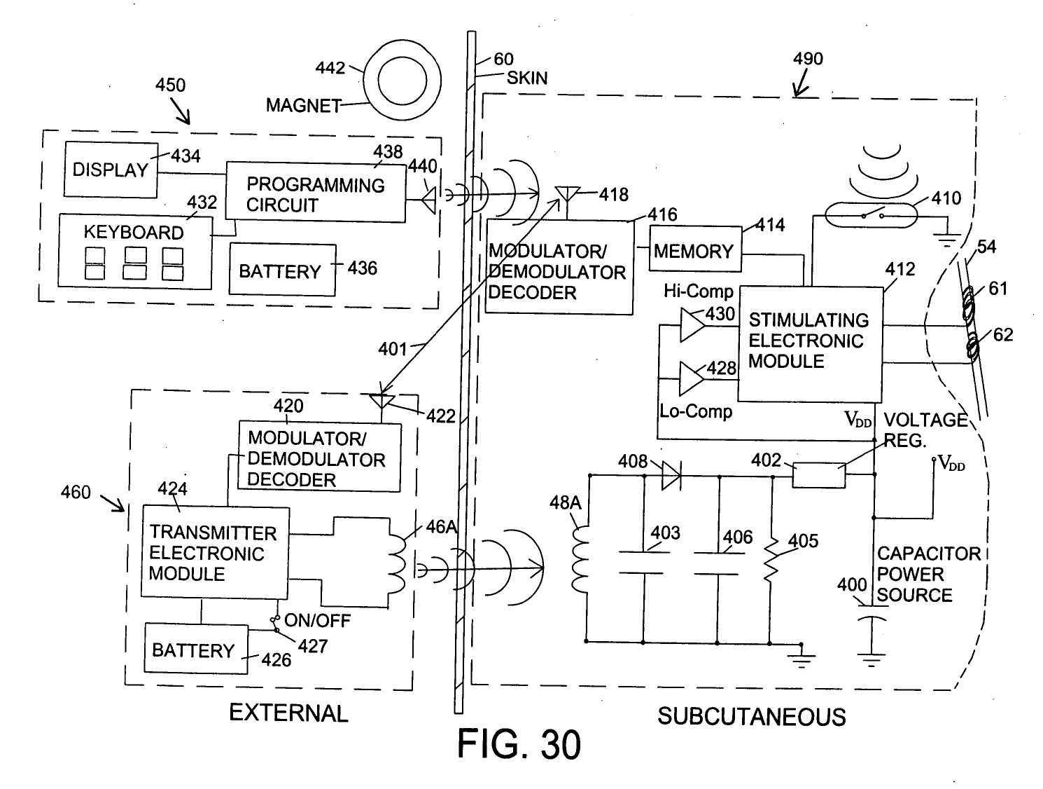

As shown in conjunction with FIG. 30 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 442 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the vagus nerve 54 via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-less Implantable Pulse Generator (IPG)

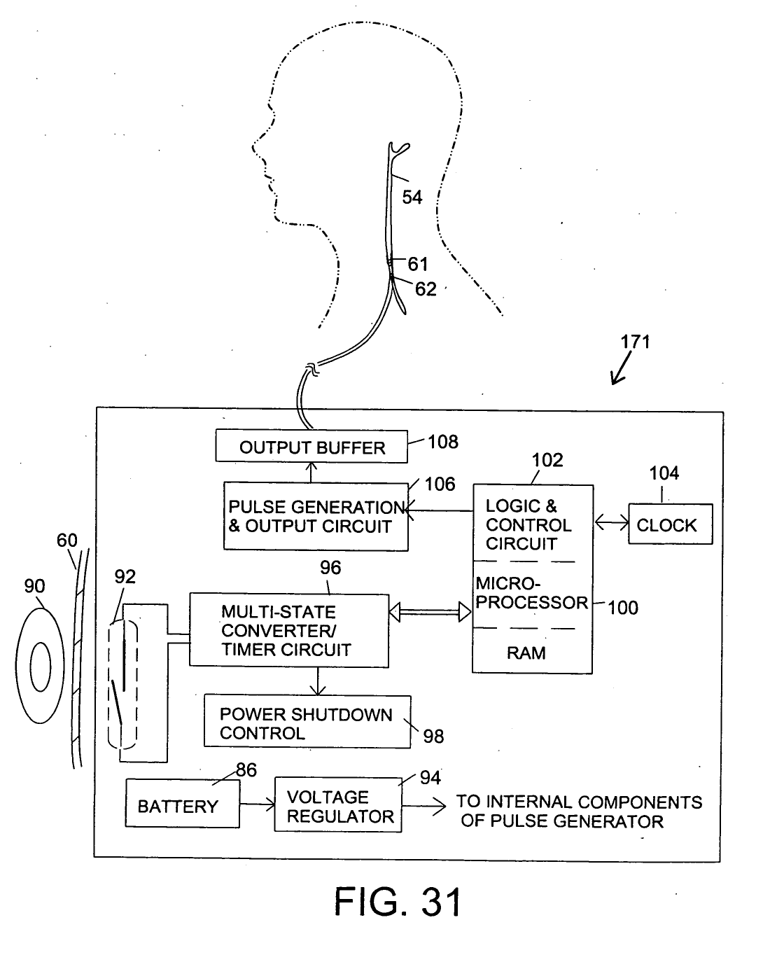

In one embodiment, a programmer-less implantable pulse generator (IPG) may be used. In this embodiment, shown in conjunction with FIG. 31, the implantable pulse generator 171 is provided with a reed switch 92 and memory circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

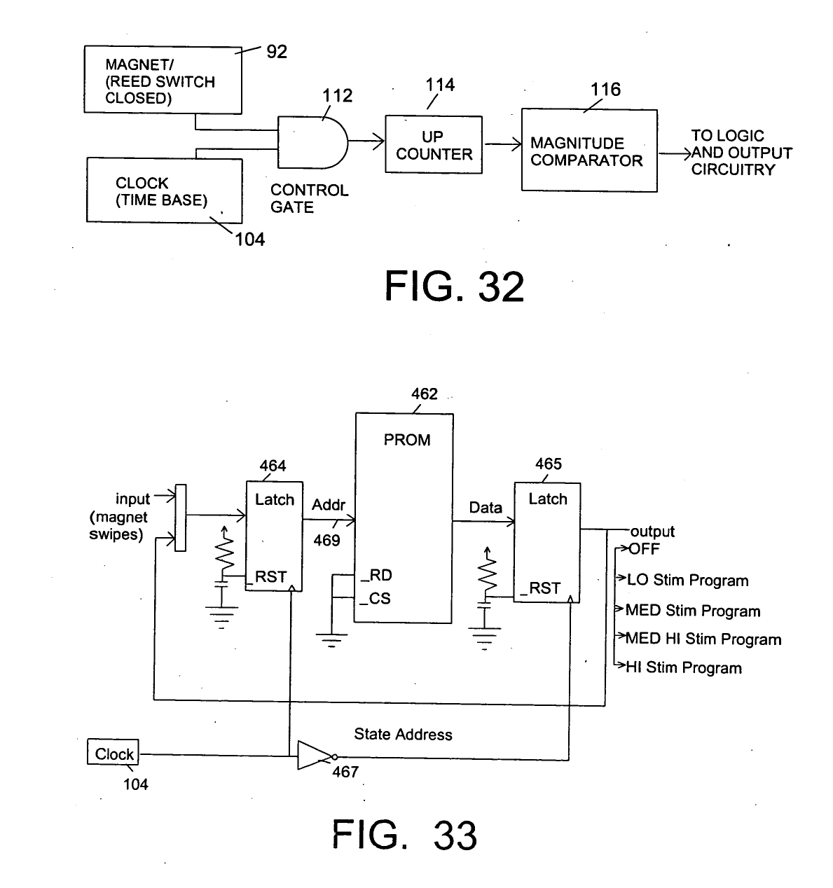

In one embodiment, shown in conjunction with FIG. 32, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, as shown in FIG. 31, the pulse generation and amplification circuit 106 deliver the appropriate electrical pulses to the vagus nerve 54 of the patient via an output buffer 108. The delivery of output pulses is configured such that the distal electrode 61 (electrode closer to the brain) is the cathode and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment without limitation, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the vagus nerve) for each state are as follows,

LOW stimulation state example is,
Current output: 0.75 milliAmps.
Pulse width: 0.20 msec.
Pulse frequency: 20 Hz
Cycles: 20 sec. on-time and 2.0 min. off-time in repeating cycles.

LOW-MED stimulation state example is,
Current output: 1.5 milliAmps,
Pulse width: 0.30 msec.
Pulse frequency: 25 Hz
Cycles: 1.5 min. on-time and 20.0 min. off-time in repeating cycles.

MED stimulation state example is,
Current output: 2.0 milliAmps.
Pulse width: 0.30 msec.
Pulse frequency: 30 Hz
Cycles: 1.5 min. on-time and 20.0 min. off-time in repeating cycles.

HIGH stimulation state example is,
Current output: 3.0 milliAmps,
Pulse width: 0.40 msec.
Pulse frequency: 30 Hz
Cycles: 2.0 min. on-time and 20.0 min. off-time in repeating cycles.

These prepackaged/predetermined programs are merely examples, and the actual stimulation parameters will deviate from these depending on the treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, triggers the device OFF.

FIG. 33 shows a representative digital circuitry used for the basic state machine circuit. The circuit consists of a PROM 462 that has part of its data fed back as a state address. Other address lines 469 are used as circuit inputs, and the state machine changes its state address on the basis of these inputs. The clock 104 is used to pass the new address to the PROM 462 and then pass the output from the PROM 462 to the outputs and input state circuits. The two latches 464, 465 are operated 180° out of phase to prevent glitches from unexpectedly affecting any output circuits when the ROM changes state. Each state responds differently according to the inputs it receives.

The advantage of this embodiment is that it is cheaper to manufacture than a fully programmable implantable pulse generator (IPG).

### Programmable Implantable Pulse Generator (IPG)

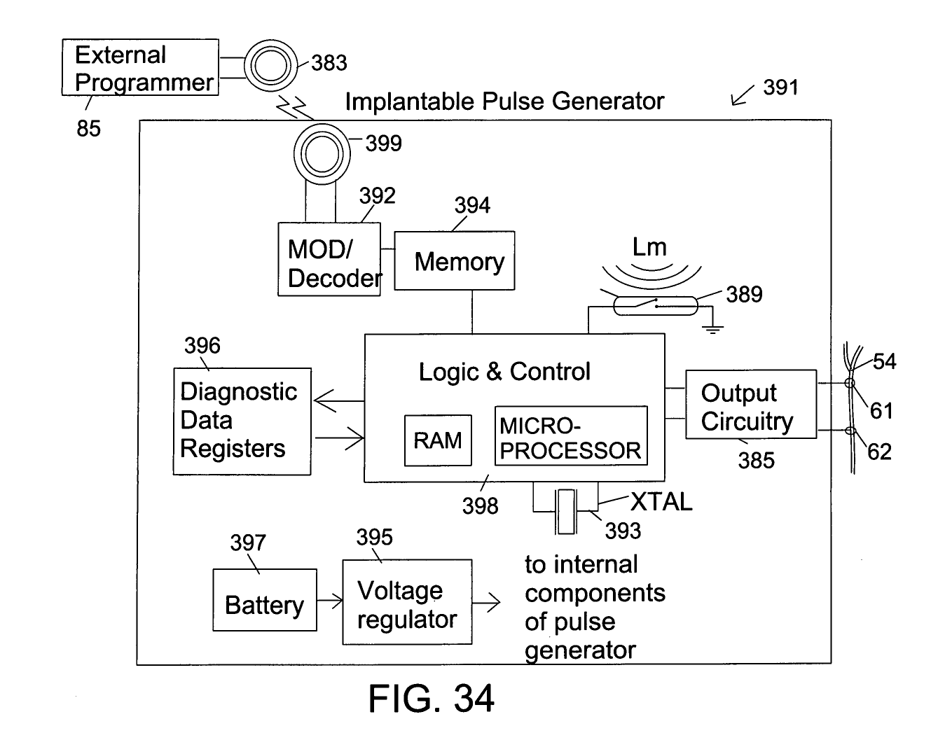

In one embodiment, a fully programmable implantable pulse generator (IPG) 391 may be used. Shown in conjunction with FIG. 34, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrodes 61, 62 via a lead 40. Programming of the implantable pulse generator (IPG) 391 is done via an external programmer 85, as described later. The external programmer may communicate with the implanted pulse generator via magnetic inductive coupling or via wireless telemetry, as described in more detail later. Once programmed via an external programmer 85, the implanted pulse generator 391 provides appropriate electrical stimulation pulses to the vagus nerve(s) 54 via electrodes 61,62.

For some applications, the implanted pulse generator (IPG) is modified to provide multiple output channels and comprises sensing. For example, in applications such as epilepsy the stimulation may be provided based on sensing from body tissues.

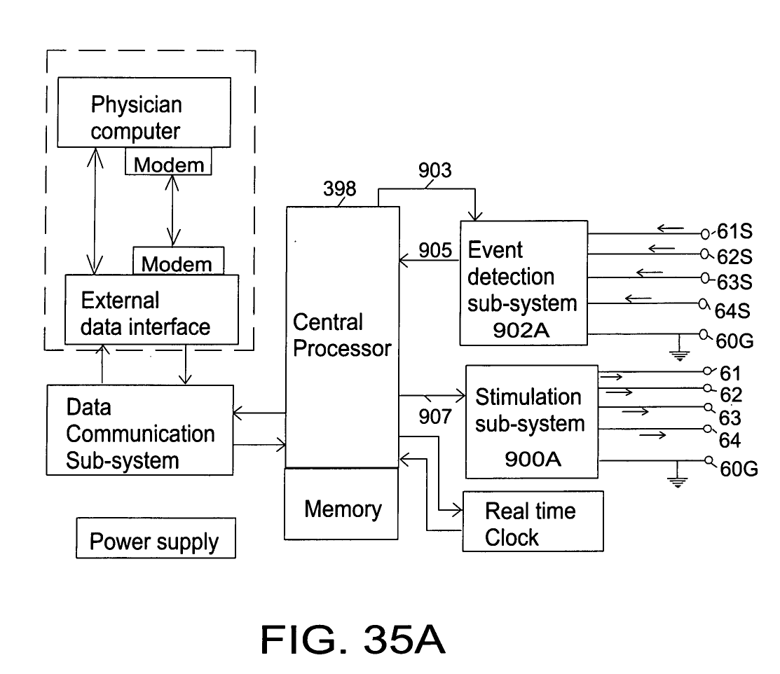

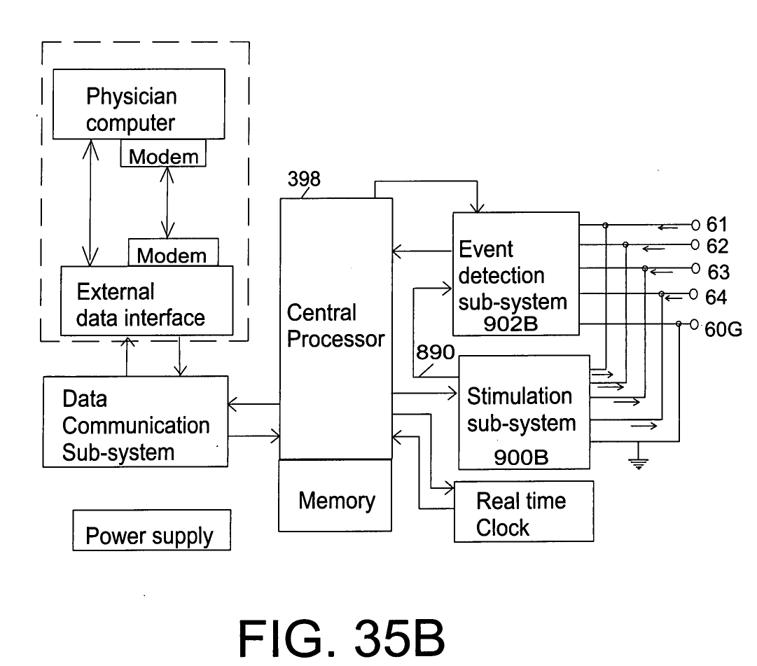

FIGS. 35A and 35B are block diagrams of the implantable system 391 and the external equipment highlighting sensing (event detection) and stimulation subsystems. Shown in FIG. 35A, the sensing function is performed utilizing dedicated electrodes (61S through 64S). In one embodiment, shown in conjunction with FIG. 35B, the sensing and stimulation functions are performed by the same electrodes utilizing the same leads. In this embodiment, it may be necessary for the stimulation sub-system 900B to temporarily disable the event detection sub-system 902B via an interconnection 890 when stimulation is imminent so that the stimulation signals are not inadvertently interpreted as a neurological event by the event detection sub-system 902B.

Referring back to FIG. 35A, the leads from the electrodes 61S through 64S and a lead from a common electrode 60G are shown connected to an event detection sub-system 902A, and the leads from the electrodes 61 through 64 and a lead from a common electrode 60G, are shown connected to a stimulation sub-system 900A. In one embodiment of the invention, it is also envisioned to use the case of the IPG as the common (or indifferent) electrode 60G. The leads carry electrogram signals from the tissues via electrodes 61S through 64S to the event detection subsystem 902A. The electrodes 61 through 64 can be energized by the stimulation sub-system 900A via the leads to electrically stimulate the patient's vagus nerve(s) using the stimulation signals.

It is envisioned that instead of electrodes 61S through 64S, one or more of the leads could instead be connected to other types of sensors. Possible additional sensors could include temperature sensors, motion sensors (accelerometers), and sensors from various tissues or organs in the body, based on impedance measurements.

The event detection sub-system 902A receives appropriate neural or myo-electrical signals (referenced to a system ground connected to the lead from the common electrode 60G) and processes them to identify events which may or may not trigger stimulation pulses. A central processing system (logic & control unit) 398 with a central processor and memory acts to control and coordinate all functions of the implantable system 391. A first interconnection 903 is used to transmit programming parameters and instruction to the event detection subsystem 902A from the central processing system 398. A second interconnection 905 is used to transmit signals to the central processing system 398 identifying the detection of a neurological event by the event detection sub-system 902A. The second interconnection 905 is also used to transmit electrogram and other related data for storage in the memory.

In this embodiment, when an event is detected by the event detection subsystem 902A (by processing), the central processor 398 can command the stimulation sub-system 900A via a third interconnection 907 to transmit electrical signals to any one or more of the electrodes 61 through 64 via the corresponding leads.

Of course, the stimulation sub-system 900A may also be engaged to perform continuous or periodic stimulation to one or more of the electrodes 61 through 64 without sensing, i.e. the sensing can be selectively turned OFF.

Referring now to FIG. 35B, it will be appreciated that electrodes 61 through 64 can be connected to both the event detection sub-system 902B and the stimulation sub-system 900B. Furthermore, it is envisioned that any one or more of the electrodes 61 through 64 could be electrically connected (i.e., shorted) to the electrode 60G or to each other. This would be accomplished by appropriate switching circuitry in the stimulation sub-system 900B.

This embodiment also comprises predetermined/pre-packaged programs. Examples were given in the previous section, under “Programmer-less Implantable Pulse Generator (IPG)”. These predetermined/pre-packaged programs comprise unique combinations of programmable parameters. Any number of predetermined/pre-packaged programs can be stored in the memory of the implantable pulse generator, and are considered within the scope of this disclosure. Without limitation, for convenience say 100, may be stored.

Examples of additional predetermined/pre-packaged programs are:

### Program A

Current output: 1.0 milliAmps.
Pulse width: 0.25 msec.
Pulse frequency: 20 Hz
Cycles: 20 sec. on-time and 3.0 min. off-time in repeating cycles.

### Program B

Current output: 1.5 milliAmps,
Pulse width: 0.40 msec.
Pulse frequency: 25 Hz
Cycles: 3.0 min. on-time and 20.0 min. off-time in repeating cycles.

### Program C

Current output: 2.0 milliAmps.
Pulse width: 0.50 msec.
Pulse frequency: 30 Hz
Cycles: 4 min. on-time and 20.0 min. off-time in repeating cycles.

### Program D

Current output: 2.5 milliAmps,
Pulse width: 0.3 msec.
Pulse frequency: 25 Hz
Cycles: 4.0 min. on-time and 20.0 min. off-time in repeating cycles.

### Program E

Current output: 3.0 milliAmps,
Pulse width: 0.50 msec.
Pulse frequency: 30 Hz
Cycles: 5.0 min. on-time and 30.0 min. off-time in repeating cycles.

These predetermined/pre-packaged programs are merely examples, and the actual stimulation/blocking parameters will deviate from these depending on the treatment application and physician preference. One advantage of predetermined/pre-packaged program is that they can be readily activated by a program number. In the method of this disclosure at least one predetermined/pre-packaged program is/are configured to cause changes in regional cerebral blood, and/or alter neurochemicals in the brain, and/or alter neural activity in the patient.

In one embodiment, the predetermined/pre-packaged program can be selectively chosen from several programs available. In another embodiment, a given predetermined/pre-packaged program can be altered or modified by changing only selected parameters. Predetermined/pre-packaged program(s) can also provide stimulation/blocking for single channel, dual channel, or multiple channels of output.

A simple version of a programmer, adapted to activate only a limited number of predetermined/pre-packaged programs may also be supplied to the patient as is described later.

A predetermined program may also be obtained by individually programming the different parameters, as determined by the physician for the individual patient. Alternatively or additionally a predetermined/pre-packaged program may be selected from memory, and selected individual parameters may then be modified or altered. The range of programmable electrical stimulation parameters are shown in table 7 below. Additionally, sensing may also be programmed ON and OFF.

TABLE 7
Programmable electrical parameter range

| PARAMETER | RANGE |
|---|---|
| Pulse Amplitude | 0.1 Volt-20 Volts |
| Pulse width | 20 μS-5 mSec. |
| Frequency | 0 Hz-5,000 Hz |
| (pulses/sec) | |
| On-time | 5 Secs-24 hours |
| Off-time | 5 Secs-24 hours |
| Ramp | ON/OFF |

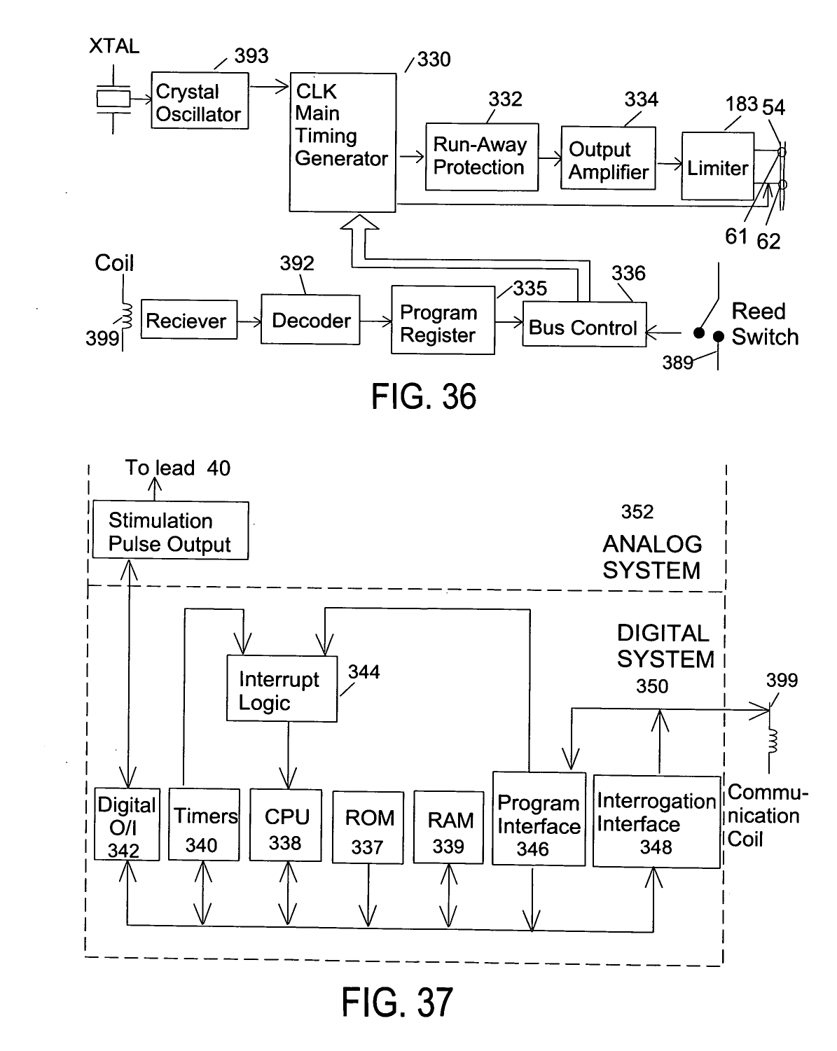

Shown in conjunction with FIGS. 36 and 37, the electronic stimulation module comprises both digital 350 and analog 352 circuits. A main timing generator 330 (shown in FIG. 36), controls the timing of the analog output circuitry for delivering neuromodulating pulses to the vagus nerve 54, via output amplifier 334. Limiter 183 prevents excessive stimulation energy from getting into the vagus nerve 54. The main timing generator 330 receiving clock pulses from crystal oscillator 393. Main timing generator 330 also receiving input from programmer 85 via coil 399. FIG. 37 highlights other portions of the digital system such as CPU 338, ROM 337, RAM 339, program interface 346, interrogation interface 348, timers 340, and digital O/I 342.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 situated under the hybrid substrate is used for bidirectional telemetry. The hybrid and battery 397 are encased in a titanium can 65. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

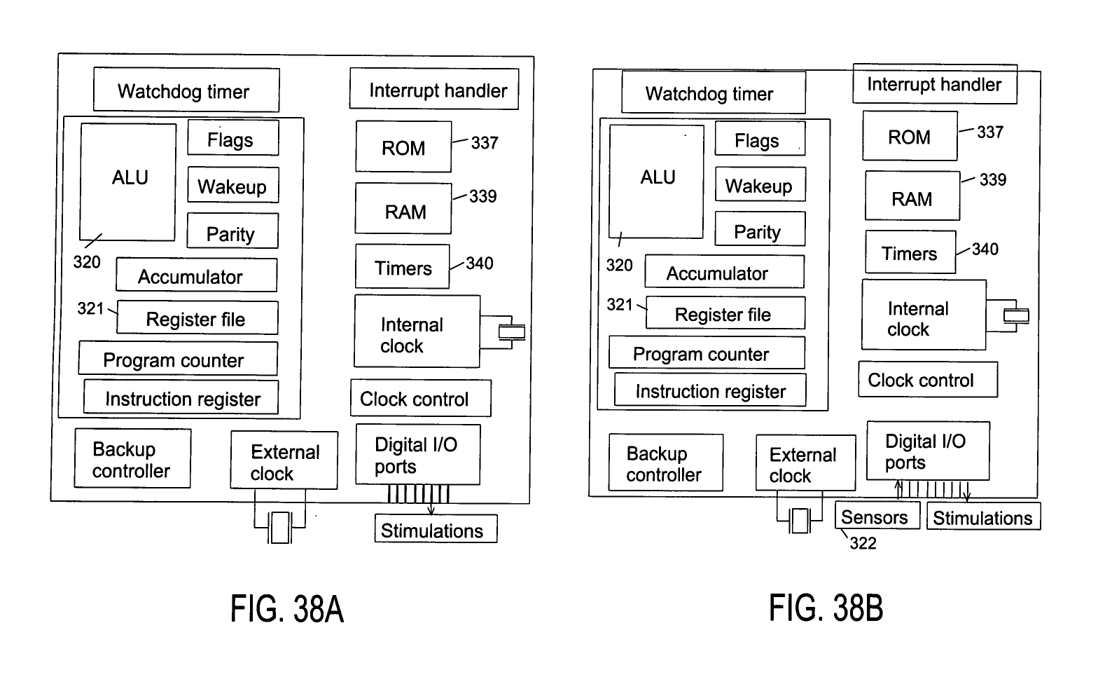

For further details, FIG. 38A highlights the general components of an 8-bit microprocessor as an example. It will be obvious to one skilled in the art that higher level microprocessor, such as a 16-bit or 32-bit may be utilized, and is considered within the scope of this invention. It comprises a ROM 337 to store the instructions of the program to be executed and various programmable parameters, a RAM 339 to store the various intermediate parameters, timers 340 to track the elapsed intervals, a register file 321 to hold intermediate values, an ALU 320 to perform the arithmetic calculation, and other auxiliary units that enhance the performance of a microprocessor-based IPG system.

The size of ROM 337 and RAM 339 units are selected based on the requirements of the algorithms and the parameters to be stored. The number of registers in the register file 321 are decided based upon the complexity of computation and the required number of intermediate values. Timers 340 of different precision are used to measure the elapsed intervals. Even though this embodiment does not have external sensors to control timing, future embodiments may have sensors 322 to effect the timing as shown in conjunction with FIG. 38B.

In this embodiment, the two main components of microprocessor are the datapath and control. The datapath performs the arithmetic operation and the control directs the datapath, memory, and I/O devices to execute the instruction of the program. The hardware components of the microprocessor are designed to execute a set of simple instructions. In general the complexity of the instruction set determines the complexity of datapath elements and controls of the microprocessor.

In this embodiment, the microprocessor is provided with a fixed operating routine. Future embodiments may be provided with the capability of actually introducing program changes in the implanted pulse generator. The instruction set of the microprocessor, the size of the register files, RAM and ROM are selected based on the performance needed and the type of the algorithms used. In this application of pulse generator, in which several algorithms can be loaded and modified, Reduced Instruction Set Computer (RISC) architecture is useful. RISC architecture offers advantages because it can be optimized to reduce the instruction cycle which in turn reduces the run time of the program and hence the current drain. The simple instruction set architecture of RISC and its simple hardware can be used to implement any algorithm without much difficulty. Since size is also a major consideration, an 8-bit microprocessor is used for the purpose. As most of the arithmetic calculation are based on a few parameters and are rather simple, an accumulator architecture is used to save bits from specifying registers. Each instruction is executed in multiple clock cycles, and the clock cycles are broadly classified into five stages: an instruction fetch, instruction decode, execution, memory reference, and write back stages. Depending on the type of the instruction, all or some of these stages are executed for proper completion.

Initially, an optimal instruction set architecture is selected based on the algorithm to be implemented and also taking into consideration the special needs of a microprocessor based implanted pulse generator (IPG). The instructions are broadly classified into Load/store instructions, Arithmetic and logic instructions (ALU), control instructions and special purpose instructions.

The instruction format is decided based upon the total number of instructions in the instruction set. The instructions fetched from memory are 8 bits long in this example. Each instruction has an opcode field (2 bits), a register specifier field (3-bits), and a 3-bit immediate field. The opcode field indicates the type of the instruction that was fetched. The register specifier indicates the address of the register in the register file on which the operations are performed. The immediate field is shifted and sign extended to obtain the address of the memory location in load/store instruction. Similarly, in branch and jump instruction, the offset field is used to calculate the address of the memory location the control needs to be transferred to.

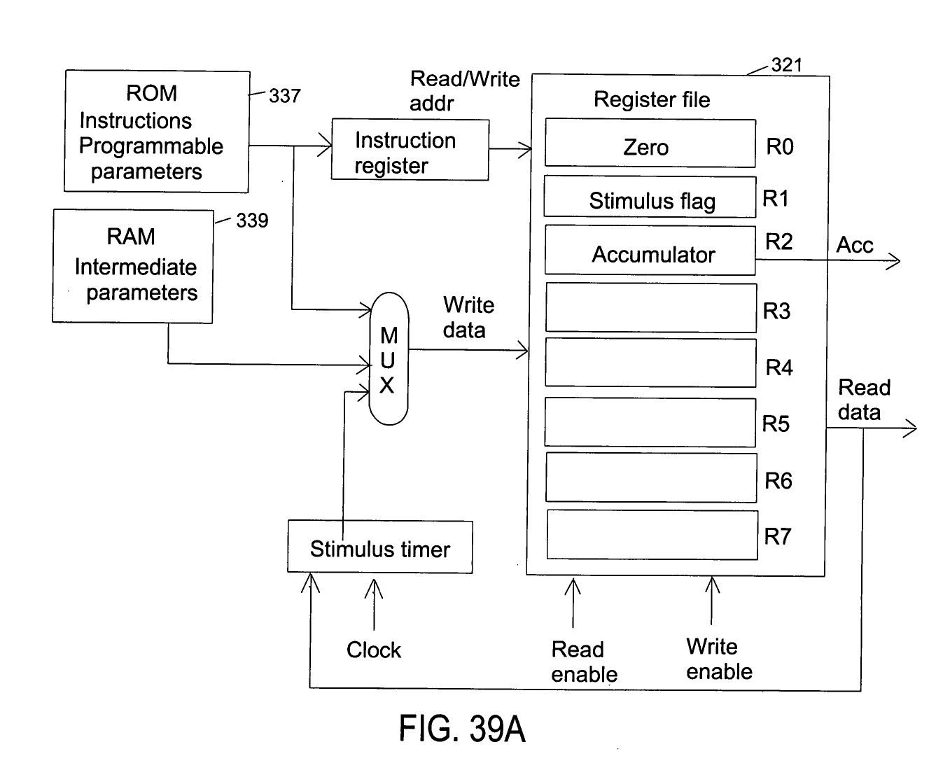

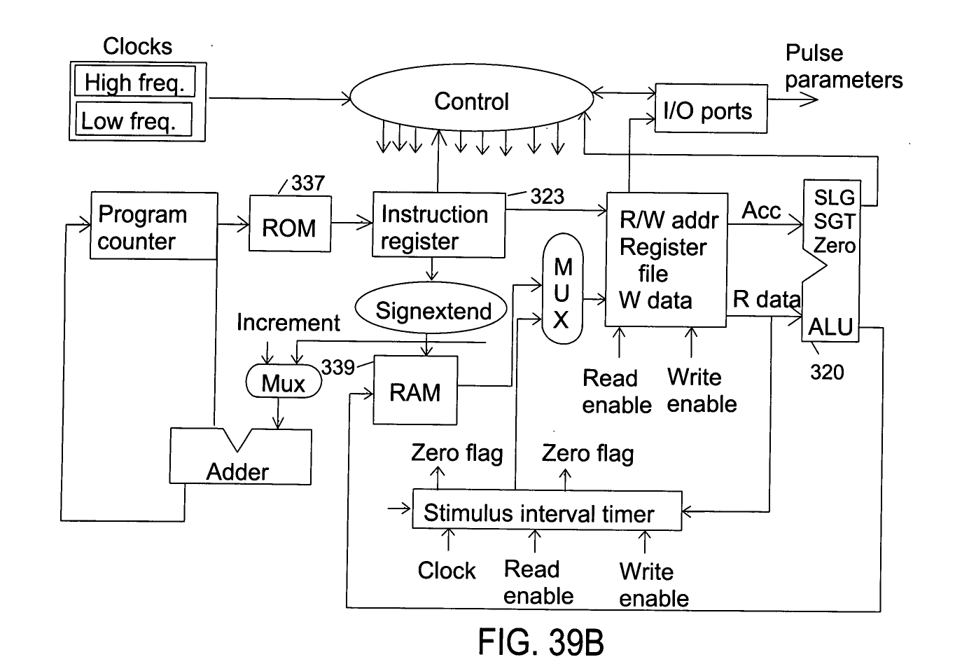

Shown in conjunction with FIG. 39A, the register file 321, which is a collection of registers in which any register can be read from or written to specifying the number of the register in the file. Based on the requirements of the design, the size of the register file is decided. For the purposes of implementation of stimulation pulses algorithms, a register file of eight registers is sufficient, with three special purpose register (0-2) and five general purpose registers (3-7), as shown in FIG. 39A. Register “0” always holds the value “zero”. Register “1” is dedicated to the pulse flags. Register “2” is an accumulator in which all the arithmetic calculations are performed. The read/write address port provides a 3-bit address to identify the register being read or written into. The write data port provides 8-bit data to be written into the registers either from ROM/RAM or timers. Read enable control, when asserted enables the register file to provide data at the read data port. Write enable control enables writing of data being provided at the write data port into a register specified by the read/write address.

Generally, two or more timers are required to implement the algorithm for the IPG. The timers are read and written into just as any other memory location. The timers are provided with read and write enable controls.

The arithmetic logic unit is an important component of the microprocessor. It performs the arithmetic operation such as addition, subtraction and logical operations such as AND and OR. The instruction format of ALU instructions consists of an opcode field (2 bits), a function field (2 bits) to indicate the function that needs to be performed, and a register specifier (3 bits) or an immediate field (4 bits) to provide an operand.

The hardware components discussed above constitute the important components of a datapath. Shown in conjunction with FIG. 39B, there are some special purpose registers such a program counter (PC) to hold the address of the instruction being fetched from ROM 337 and instruction register (IR) 323, to hold the instruction that is fetched for further decoding and execution. The program counter is incremented in each instruction fetch stage to fetch sequential instruction from memory. In the case of a branch or jump instruction, the PC multiplexer allows to choose from the incremented PC value or the branch or jump address calculated. The opcode of the instruction fetched (IR) is provided to the control unit to generate the appropriate sequence of control signals, enabling data flow through the datapath. The register specification field of the instruction is given as read/write address to the register file, which provides data from the specified field on the read data port. One port of the ALU is always provided with the contents of the accumulator and the other with the read data port. This design is therefore referred to as accumulator-based architecture. The sign-extended offset is used for address calculation in branch and jump instructions. The timers are used to measure the elapsed interval and are enabled to count down on a low-frequency clock. The timers are read and written into, just as any other memory location (FIG. 39B).

In a multicycle implementation, each stage of instruction execution takes one clock cycle. Since the datapath takes multiple clock cycles per instruction, the control must specify the signals to be asserted in each stage and also the next step in the sequence. This can be easily implemented as a finite state machine.

A finite state machine consists of a set of states and directions on how to change states. The directions are defined by a next-state function, which maps the current state and the inputs to a new state. Each stage also indicates the control signals that need to be asserted. Every state in the finite state machine takes one clock cycle. Since the instruction fetch and decode stages are common to all the instruction, the initial two states are common to all the instruction. After the execution of the last step, the finite state machine returns to the fetch state.

A finite state machine can be implemented with a register that holds the current stage and a block of combinational logic such as a PLA. It determines the datapath signals that need to be asserted as well as the next state. A PLA is described as an array of AND gates followed by an array of OR gates. Since any function can be computed in two levels of logic, the two-level logic of PLA is used for generating control signals.

The occurrence of a wakeup event initiates a stored operating routine corresponding to the event. In the time interval between a completed operating routine and a next wake up event, the internal logic components of the processor are deactivated and no energy is being expended in performing an operating routine.

A further reduction in the average operating current is obtained by providing a plurality of counting rates to minimize the number of state changes during counting cycles. Thus intervals which do not require great precision, may be timed using relatively low counting rates, and intervals requiring relatively high precision, such as stimulating pulse width, may be timed using relatively high counting rates.

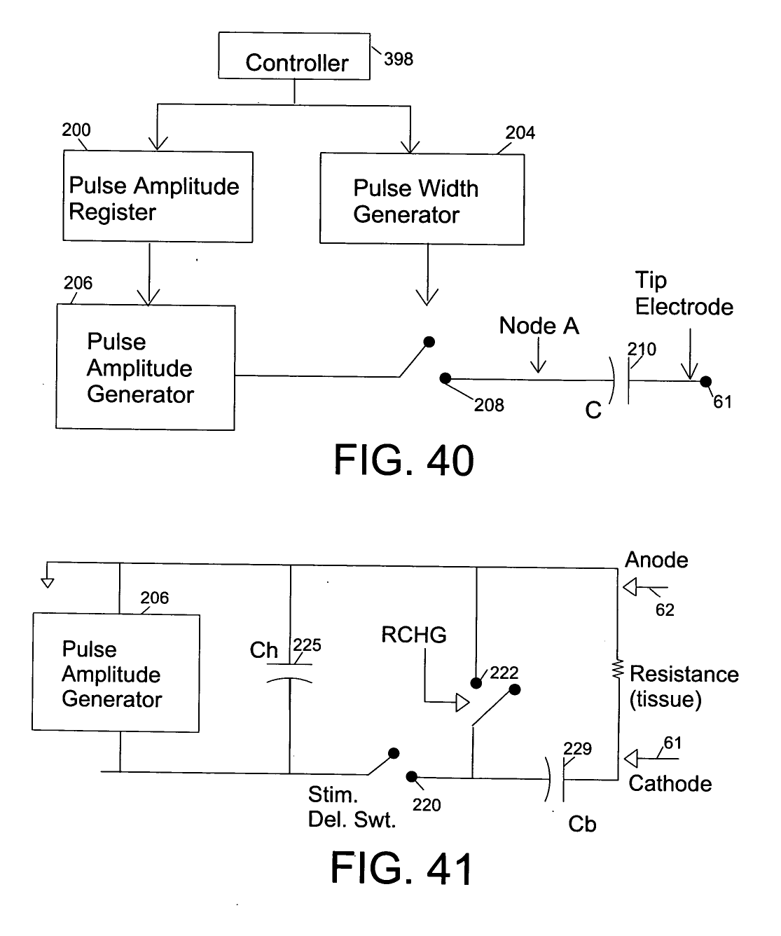

The logic and control unit 398 of the IPG controls the output amplifiers. The pulses have predetermined energy (pulse amplitude and pulse width) and are delivered at a time determined by the therapy stimulus controller. The circuitry in the output amplifier, shown in conjunction with (FIG. 40) generates an analog voltage or current that represents the pulse amplitude. The stimulation controller module initiates a stimulus pulse by closing a switch 208 that transmits the analog voltage or current pulse to the nerve tissue through the tip electrode 61 of the lead 40. The output circuit receiving instructions from the stimulus therapy controller 398 that regulates the timing of stimulus pulses and the amplitude and duration (pulse width) of the stimulus. The pulse amplitude generator 206 determines the configuration of charging and output capacitors necessary to generate the programmed stimulus amplitude. The output switch 208 is closed for a period of time that is controlled by the pulse width generator 204. When the output switch 208 is closed, a stimulus is delivered to the tip electrode 61 of the lead 40.

The constant-voltage output amplifier applies a voltage pulse to the distal electrode (cathode) 61 of the lead 40. A typical circuit diagram of a voltage output circuit is shown in FIG. 41. This configuration contains a stimulus amplitude generator 206 for generating an analog voltage. The analog voltage represents the stimulus amplitude and is stored on a holding capacitor Cₕ 225. Two switches are used to deliver the stimulus pulses to the lead 40, a stimulating delivery switch 220, and a recharge switch 222, that reestablishes the charge equilibrium after the stimulating pulse has been delivered to the nerve tissue. Since these switches have leakage currents that can cause direct current (DC) to flow into the lead system 40, a DC blocking capacitor C_{b} 229, is included. This is to prevent any possible corrosion that may result from the leakage of current in the lead 40. When the stimulus delivery switch 220 is closed, the pulse amplitude analog voltage stored in the (Cₕ 225) holding capacitor is transferred to the cathode electrode 61 of the lead 40 through the coupling capacitor, C_{b} 229. At the end of the stimulus pulse, the stimulus delivery switch 220 opens. The pulse duration being the interval from the closing of the switch 220 to its reopening. During the stimulus delivery, some of the charge stored on Cₕ 225 has been transferred to C_{b} 229, and some has been delivered to the lead system 40 to stimulate the nerve tissue.

To re-establish equilibrium, the recharge switch 222 is closed, and a rapid recharge pulse is delivered. This is intended to remove any residual charge remaining on the coupling capacitor C_{b} 229, and the stimulus electrodes on the lead (polarization). Thus, the stimulus is delivered as the result of closing and opening of the stimulus delivery 220 switch and the closing and opening of the RCHG switch 222. At this point, the charge on the holding Cₕ 225 must be replenished by the stimulus amplitude generator 206 before another stimulus pulse can be delivered.

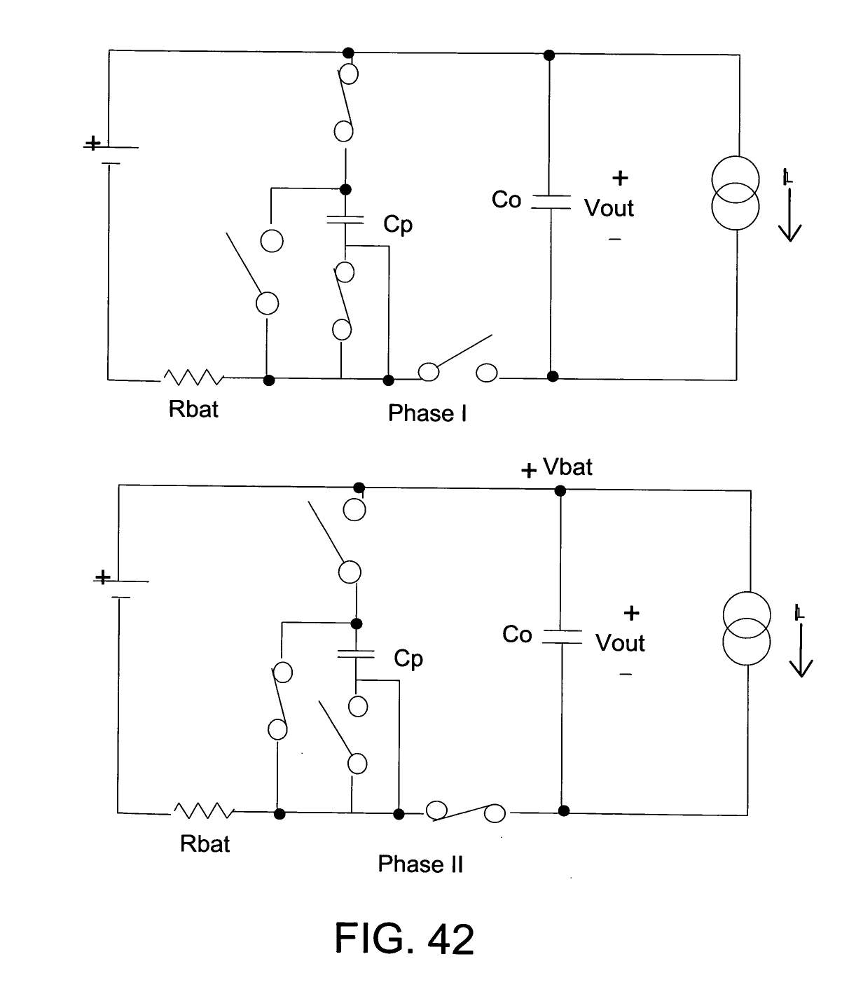

The pulse generating unit charges up a capacitor and the capacitor is discharged when the control (timing) circuitry requires the delivery of a pulse. This embodiment utilizes a constant voltage pulse generator, even though a constant current pulse generator can also be utilized. Pump-up capacitors are used to deliver pulses of larger magnitude than the potential of the batteries. The pump up capacitors are charged in parallel and discharged into the output capacitor in series. Shown in conjunction with FIG. 42 is a circuit diagram of a voltage doubler which is shown here as an example. For higher multiples of battery voltage, this doubling circuit can be cascaded with other doubling circuits. As shown in FIG. 42, during phase I (top of FIG. 42), the pump capacitor Cₚ is charged to V_{bat} and the output capacitor Cₒ supplies charge to the load. During phase II, the pump capacitor charges the output capacitor, which is still supplying the load current. In this case, the voltage drop across the output capacitor is twice the battery voltage.

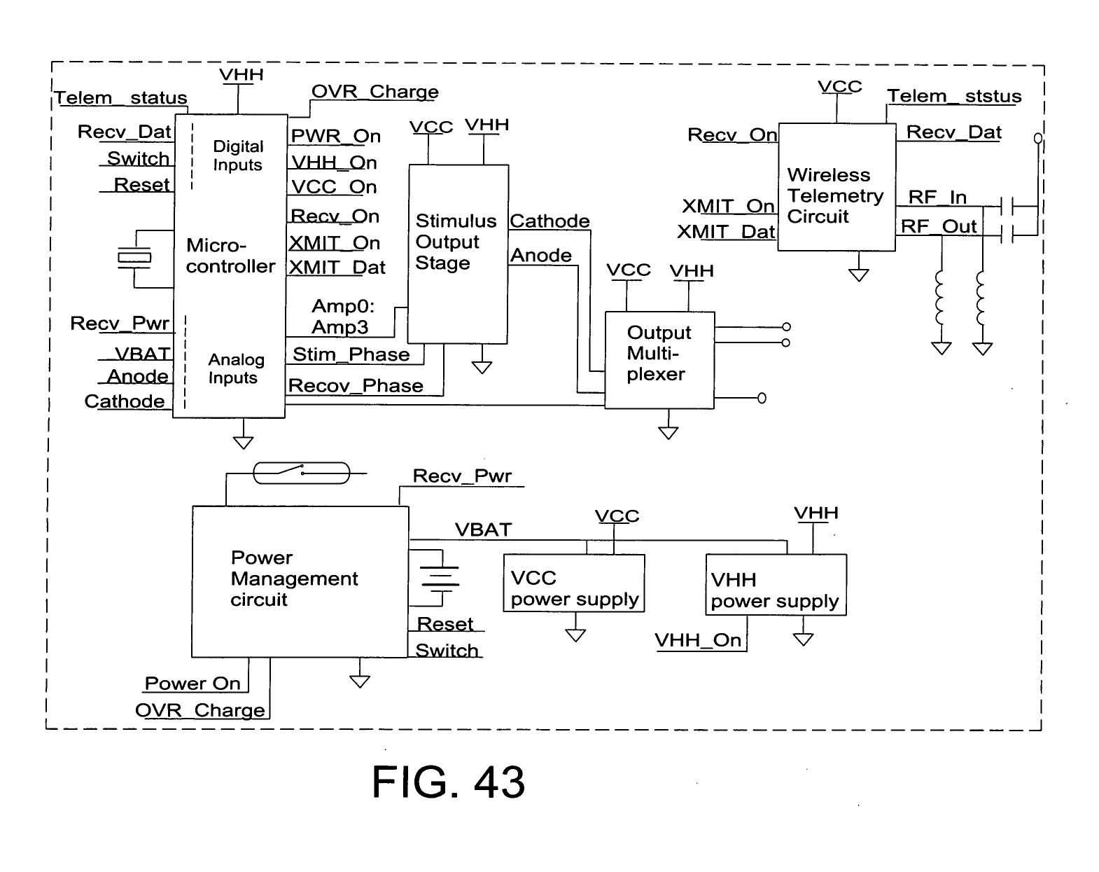

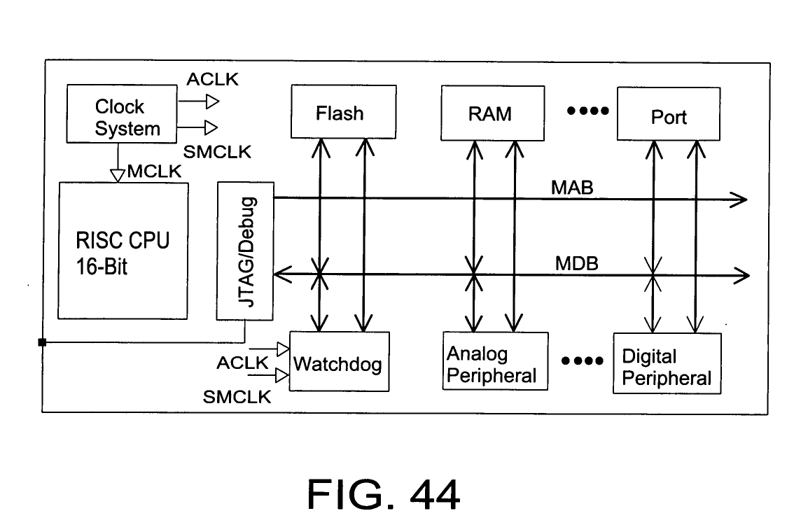

FIG. 43 shows a representative workable implantable pulse generator (IPG) circuitry where a single chip microcontroller is used, which is a member of the Texas Instruments MSP430 family of flash programmable micro-power, highly integrated mixed signal microcontroller. A Block diagram of this chip (Texas Instruments MSP430 microcontroller) is shown in FIG. 44. Other family members of this microcontroller chip such as MSP430F168, MSP430F169 or other family members may also be used.

The circuitry shown in FIG. 43 utilizes wireless telemetry with a micropower transceiver chip (such as the AMI Semiconducter AMIS-52100) for communicating with a programmer or a patient controller which is external to the body. In some embodiments, the communication between the programmer and the implanted pulse generator (IPG) may be via magnetic inductive coupling. In other embodiments, the communication between the IPG and external programmer may utilize wireless telemetry (as shown in FIG. 43). Both are within the scope of this disclosure, and are highlighted in later sections.

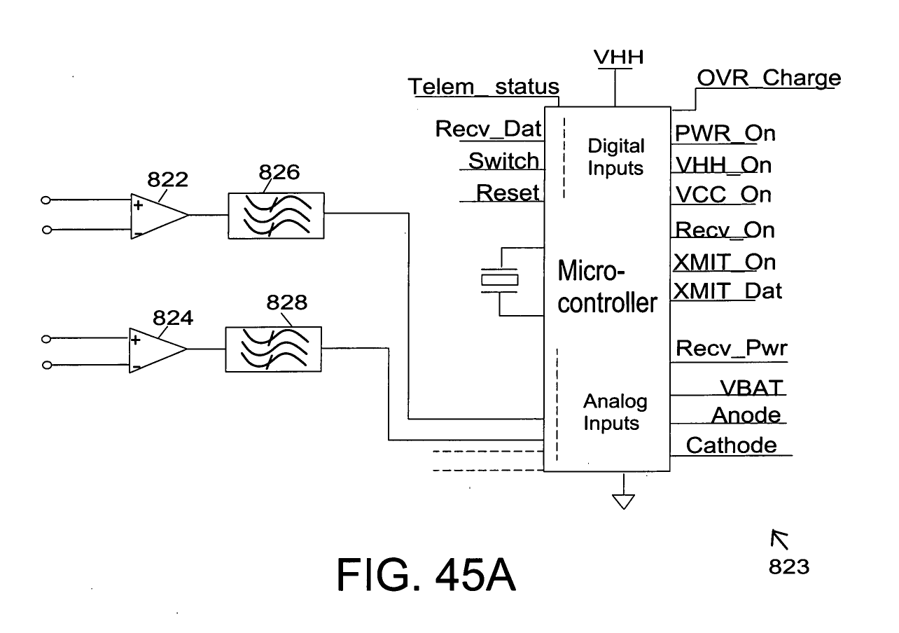

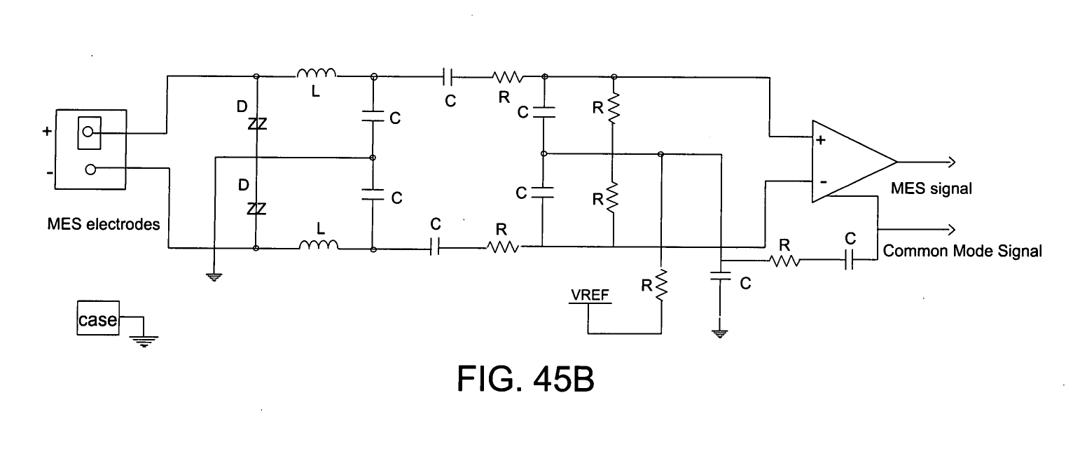

As was previously mentioned, some embodiments may utilize sensing from the body tissues and incorporate processing of sensed signals to provide electrical stimulation therapy. Shown in conjunction with FIG. 45A is amplifier 822, 824 and filtering 826, 828 circuitry connected to the analog inputs of the microcontroller 823 as was shown in FIG. 43. Amplifier circuitry is well known in the art, and a representative amplifier circuit is shown in FIG. 45B.

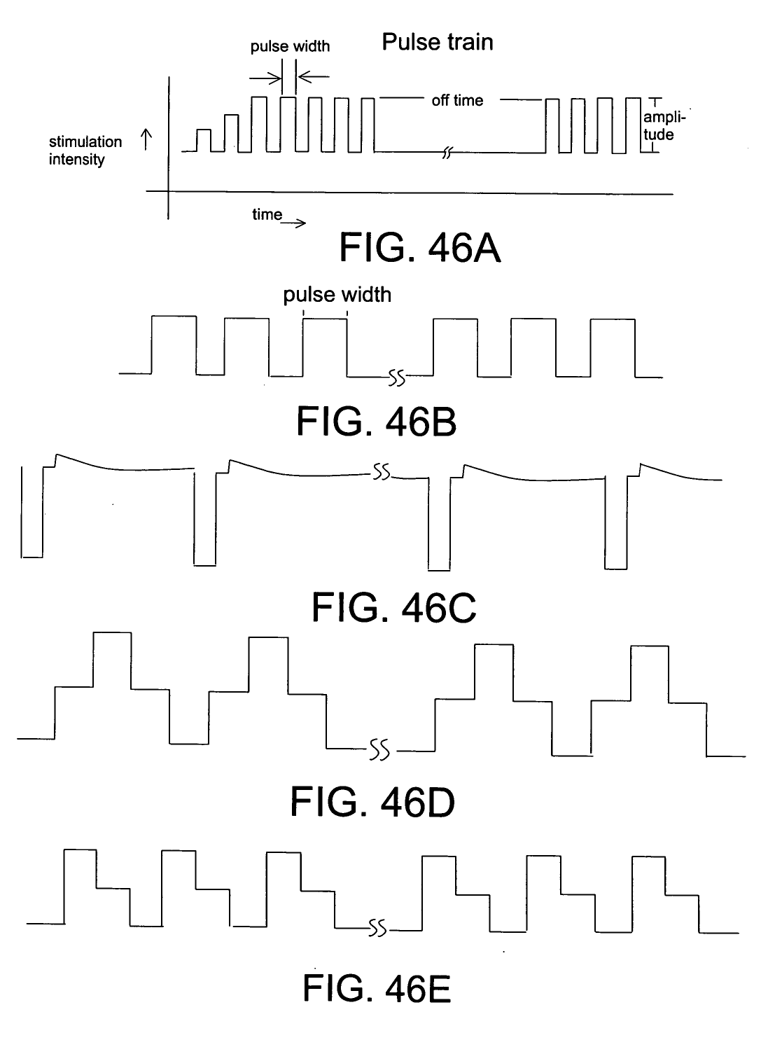

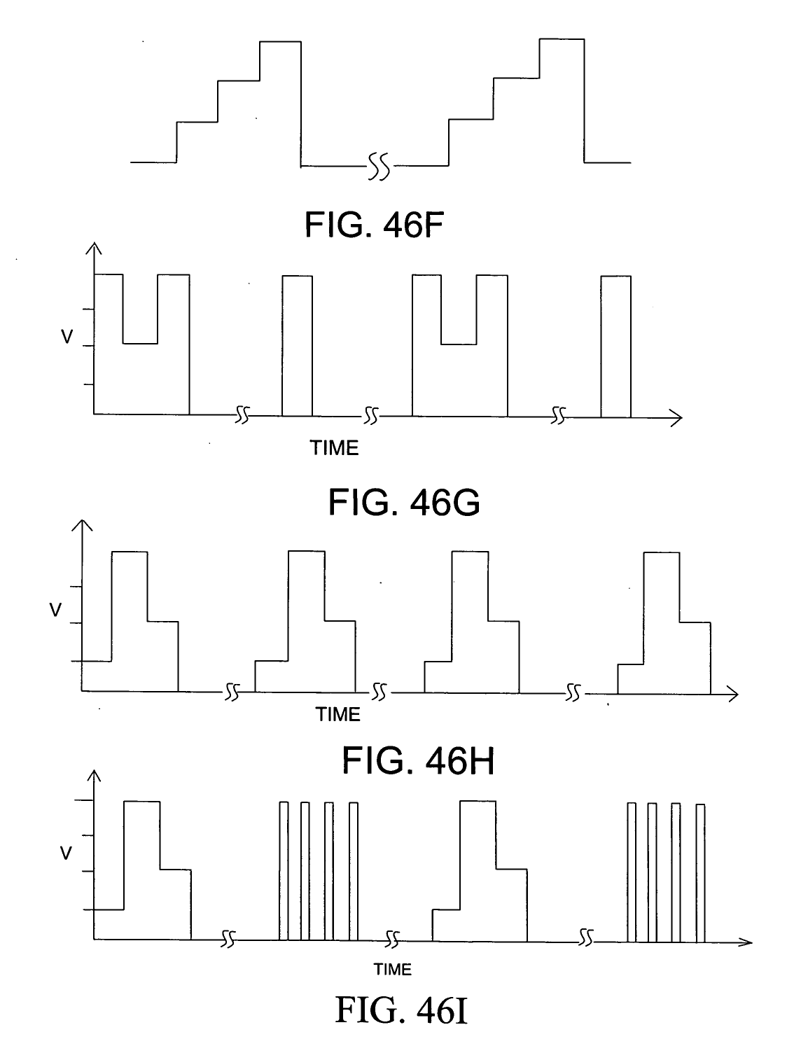

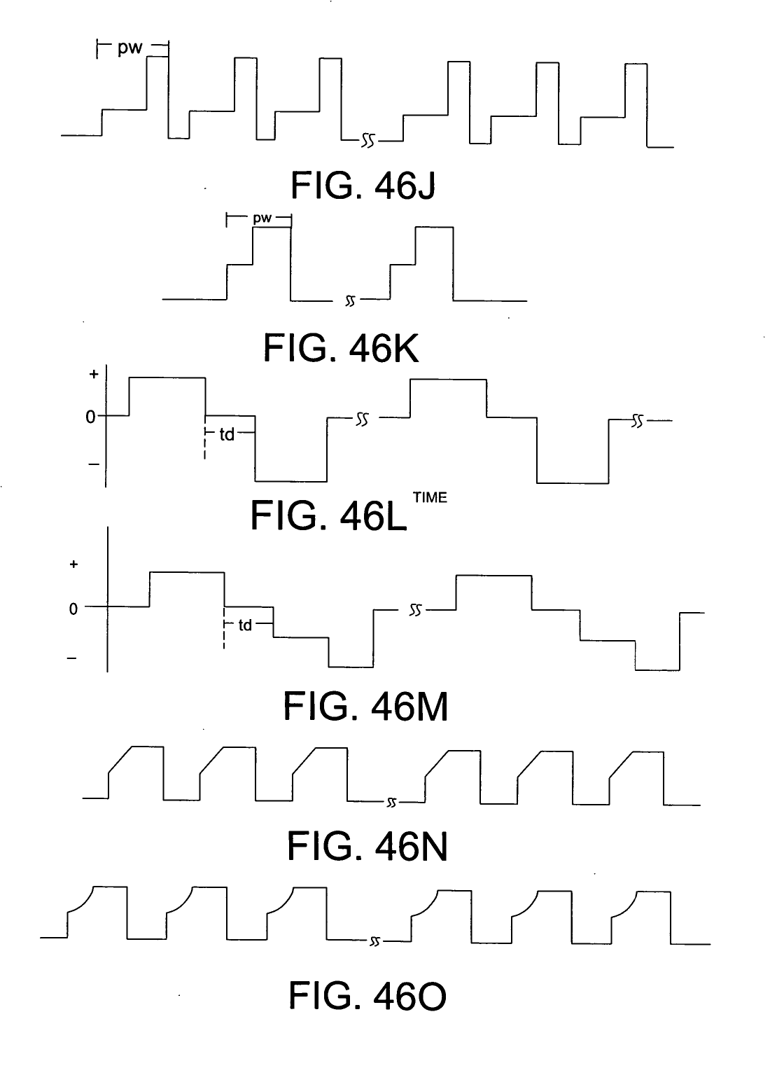

FIG. 46A shows one example of the pulse trains that may be delivered with this embodiment of vagus nerve stimulator. The microcontroller may be configured to deliver the pulse train as shown in the figure, i.e. “ramping up” of the pulse train. The purpose of the ramping-up is to avoid sudden changes in stimulation, when the pulse train begins. The ramping-up or ramping-down is optional, and may be programmed into the microcontroller.

FIG. 46B depicts rectangular pulses. FIG. 46C depicts biphasic stimulus pulses which may provide a interphase delay between the cathodic phase and anodic (charge recovery) phase.

The prior art systems delivering fixed rectangular pulses provide limited capability for selective stimulation or neuromodulation of vagus nerve(s). A fixed rectangular pulse, whether constant voltage or constant current, will recruit either i) A-fibers, or ii) A and B fibers, or iii) A and B and C fibers. Only one of these three discrete states can be achieved. This form of modulation is limited for providing therapy for neurological and neuropsychiatric disorders.

In the method and system of the current disclosure, the microcontroller is configured to deliver complex pulses. Complex pulses comprise rectangular, non-rectangular, biphasic, multi-step, and other complex pulses where the amplitude is varying during the pulse. Advantageously, these complex pulses provide a new dimension to neuromodulation of vagus nerve(s) to provide therapy for neurological disorders and neuropsychiatric disorders.

Examples of these pulses and pulse trains are shown in FIGS. 46D to 46-I. Neuromodulation with these complex pulses takes into account the threshold properties of different types of nerve fibers, as well as, the different refractory properties of different types of nerve fibers that are contained in the vagus nerve(s).

For example in the multi-step pulse shown in FIG. 46D, the first part of the pulse will tend to recruit large diameter (and myelinated) fibers, such as A and B fibers. The middle portion of the pulse where the amplitude is highest, will tend to recruit C-fibers which are the smallest fibers, and the last portion of the pulse will again tend to recruit the large diameter fibers provided they are not refractory. The multi-step (and multi-amplitude) pulses shown in FIG. 46F will tend to recruit large diameter fibers initially, and the later part of the pulse will tend to recruit the smaller diameter C-fibers.

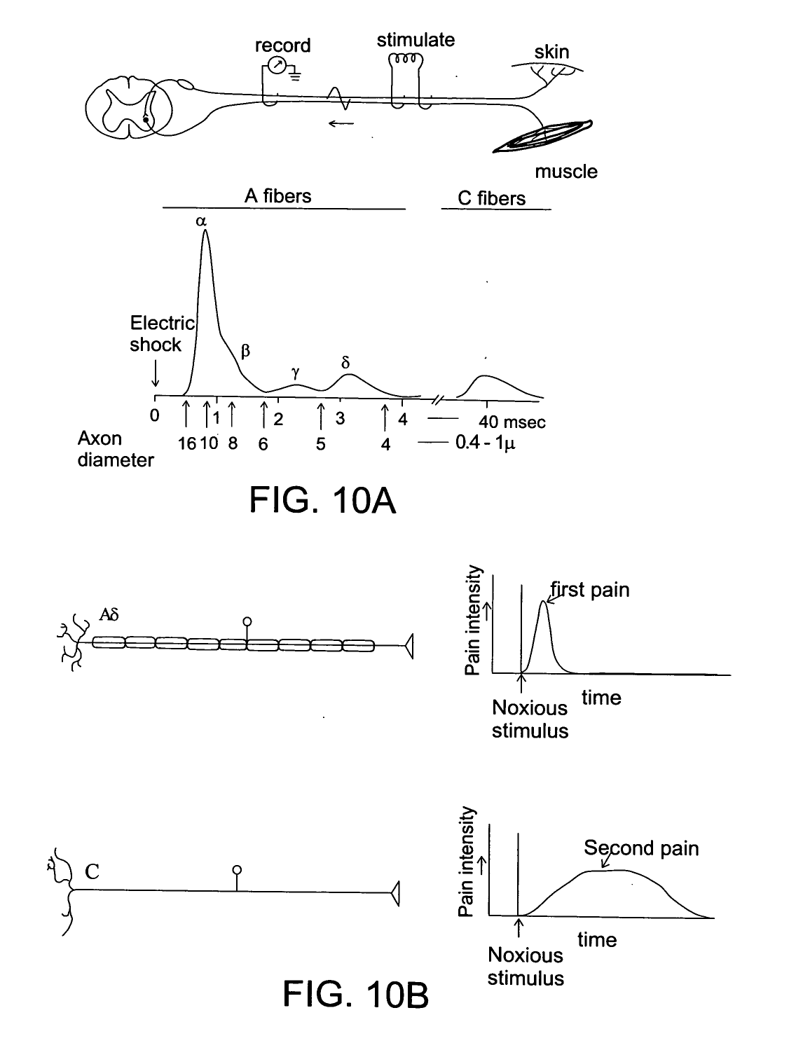

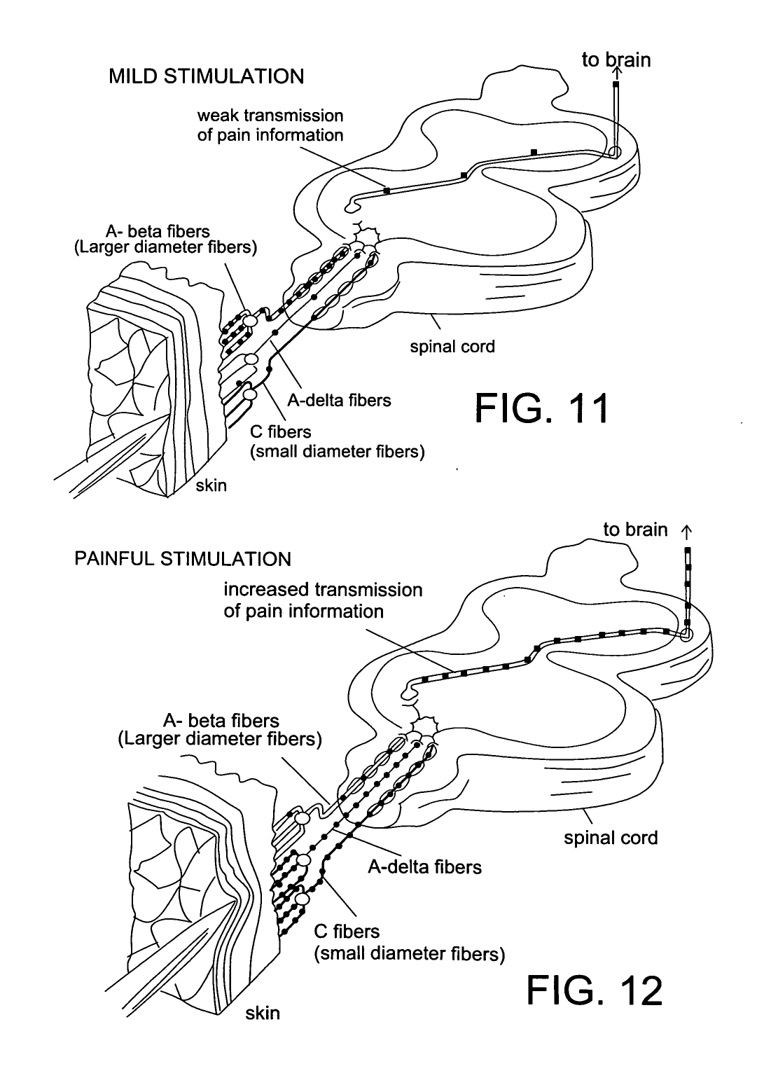

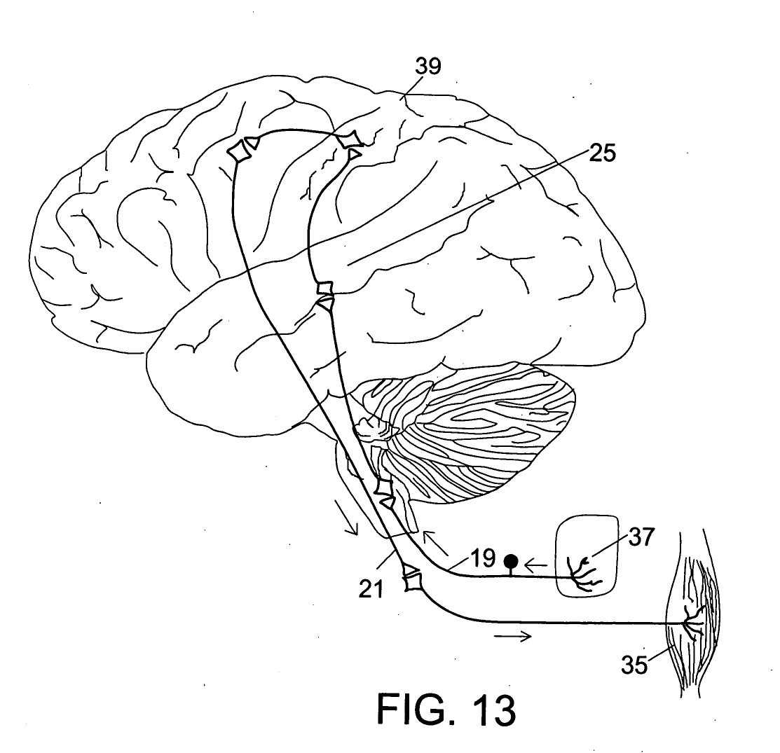

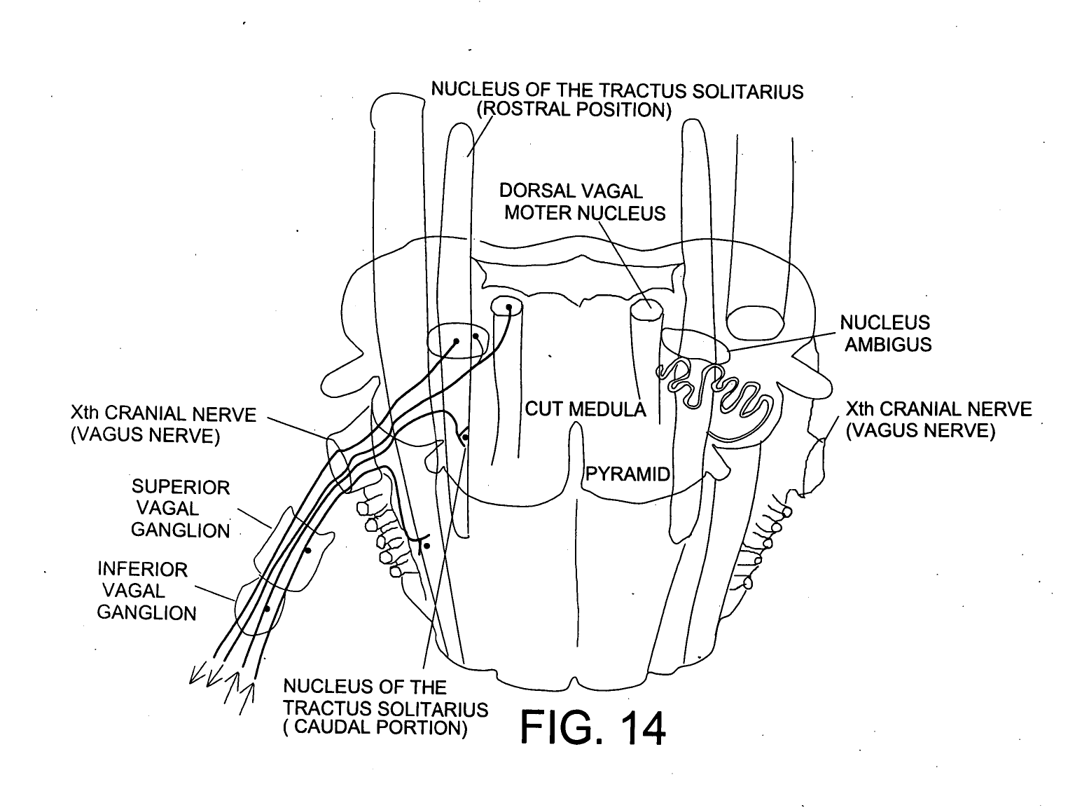

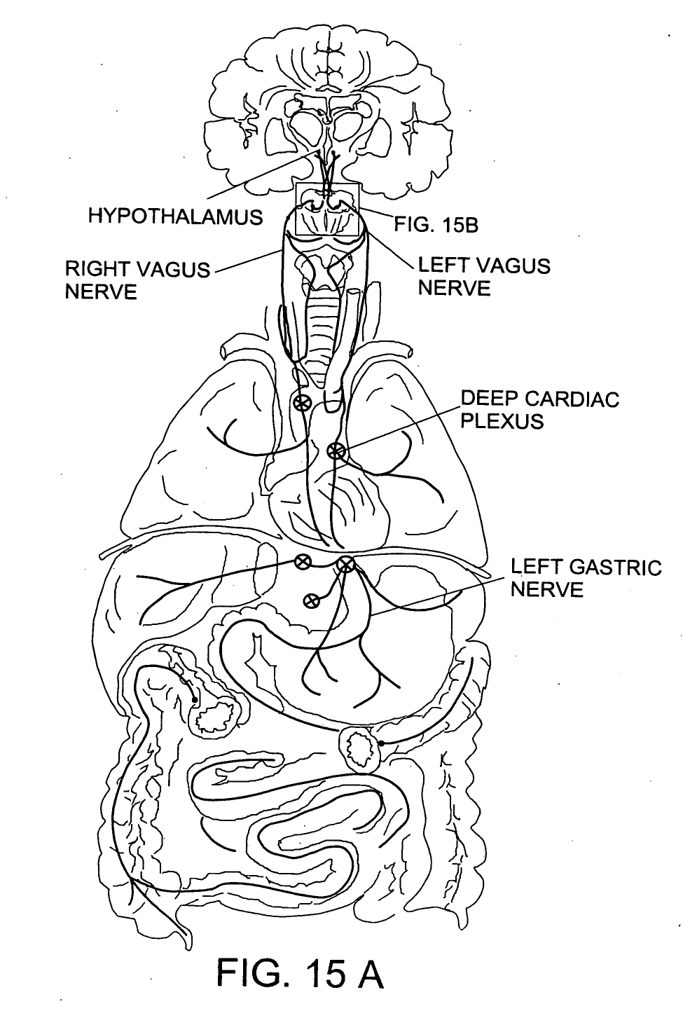

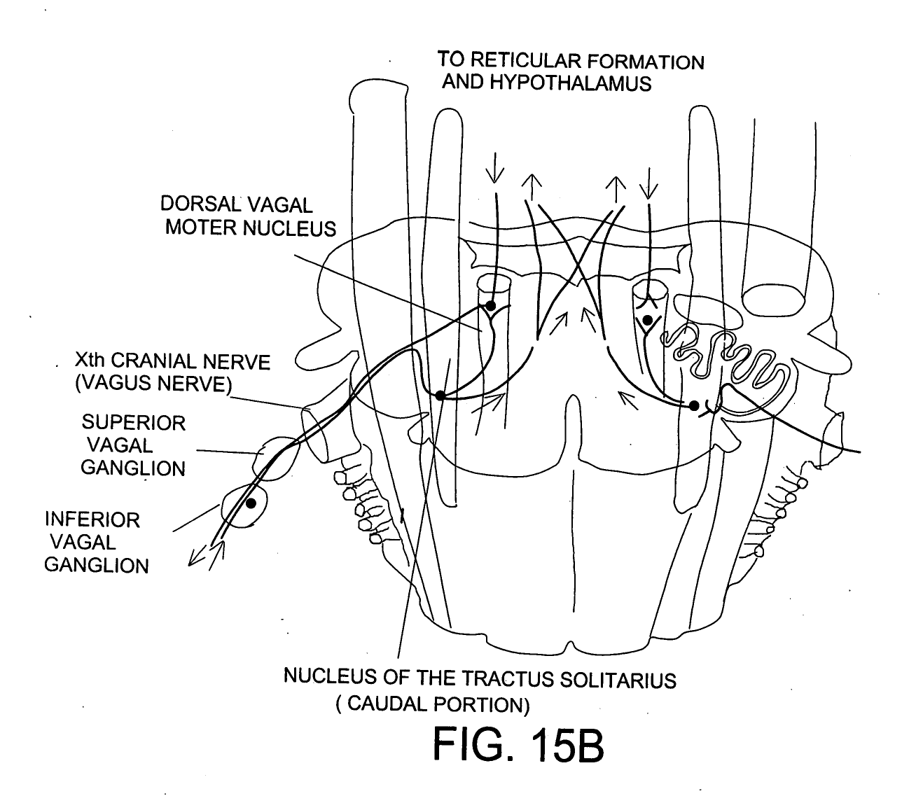

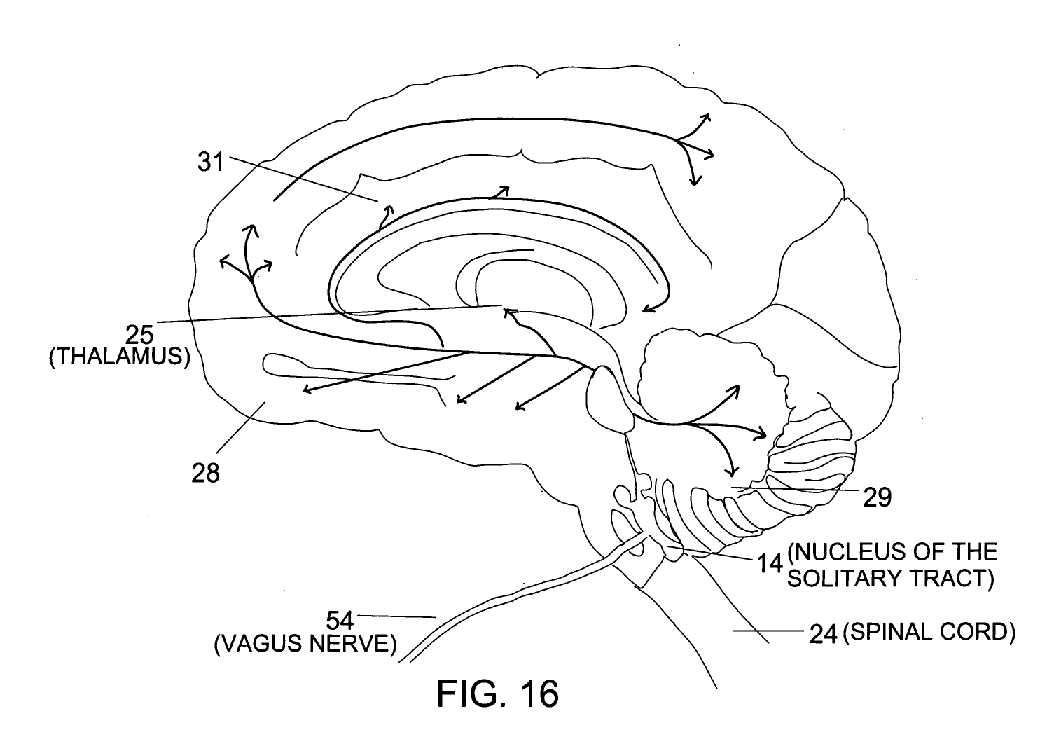

Further, as shown in the examples of FIGS. 46G and 46-I, complex and simple pulses, or pulse trains may be alternated. It will be clear to one skilled in the art, that the pulse trains in these two examples take into account both the threshold properties and the refractory properties of different types of nerve fibers which were shown in FIGS. 2 and 10A. FIGS. 46J-46O show additional examples of complex pulses that may be utilized.

The pulses and pulse trains of this disclosure gives physicians a lot of flexibility for trying various different neuromodulation algorithms for providing and optimizing therapy for neurologic and neuropsychiatric disorders.

Since the number of types of pulses and pulse trains to provide therapy can be complex for many physician's, in one aspect pre-determined/pre-packaged program comprise a complete program for the pulse trains that deliver therapy. The advantage of the pre-packaged programs is that the physician may program a complicated program simply by selecting a program number.

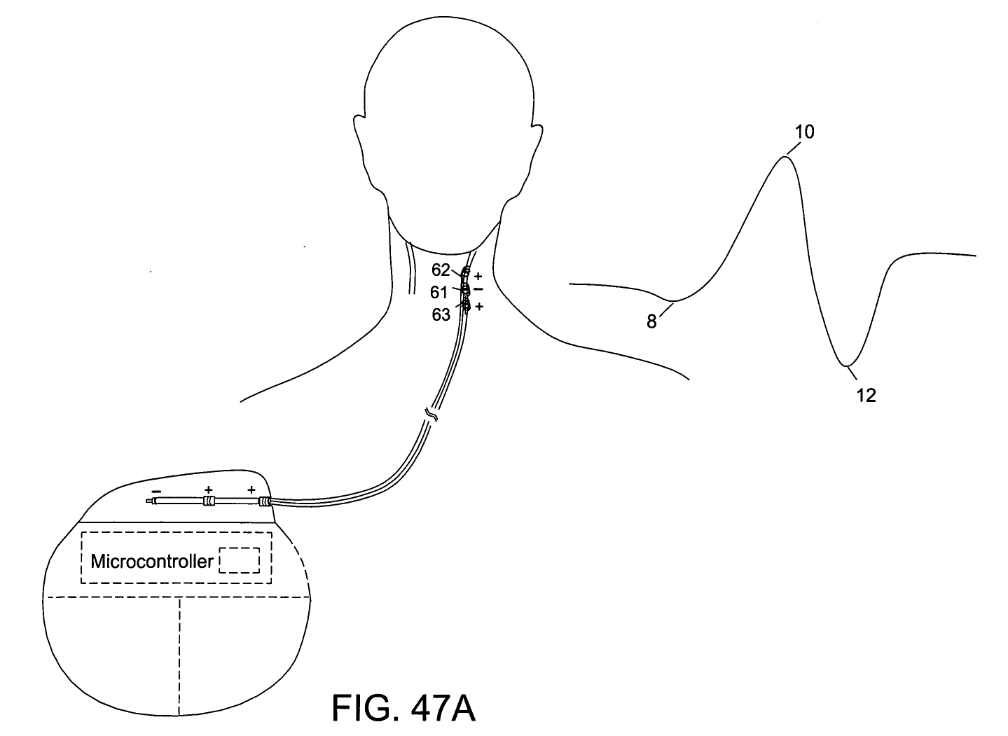

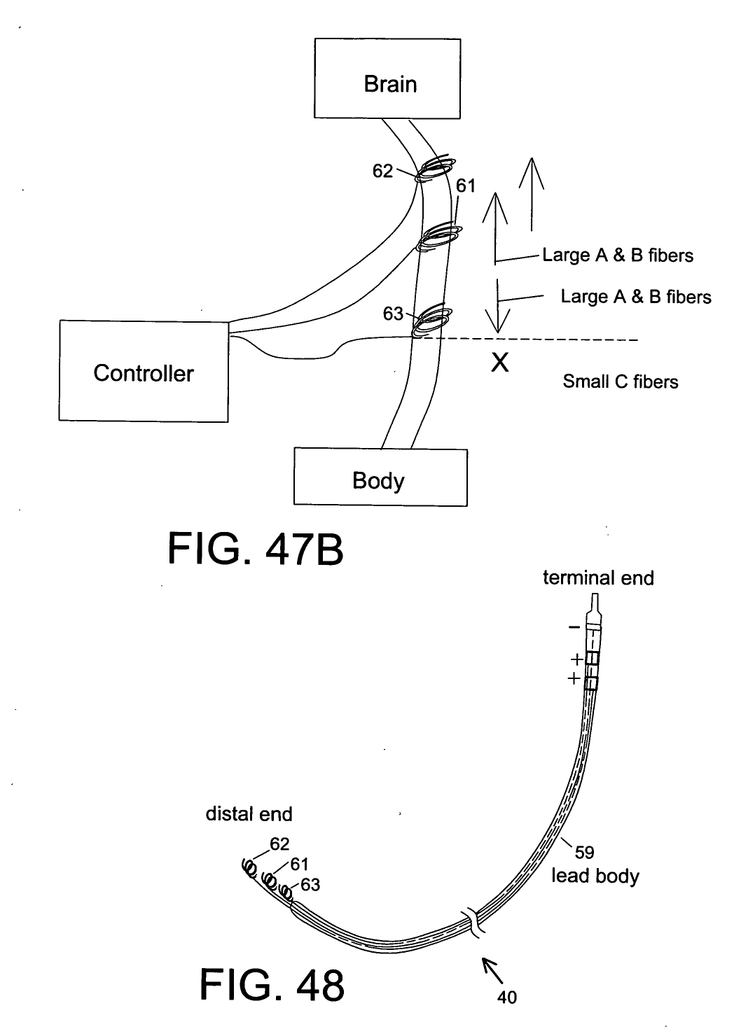

Since one of the objectives of this disclosure is to deliver afferent stimulation, in one aspect efferent stimulation of selected types of fibers may be substantially blocked, utilizing the “greenwave” effect. In such a case, as shown in conjunction with FIGS. 47A and 47B, a tripolar lead is utilized. As depicted on the top right portion of FIG. 47A, a depolarization peak 10 on the vagus nerve bundle corresponding to electrode 61 (cathode) and the two hyper-polarization peaks 8, 12 corresponding to electrodes 62, 63 (anodes). With the microcontroller controlling the tripolar device, the size and timing of the hyper-polarizations 8, 12 can be controlled. As was shown previously in FIGS. 2 and 10A, since the speed of conduction is different between the larger diameter A and B fibers and the smaller diameter c-fibers, by appropriately timing the pulses, collision blocks can be created for conduction via the large diameter A and B fibers in the efferent direction. This is depicted schematically in FIG. 47B. A lead with tripolar electrodes for stimulation/blocking is shown in conjunction with FIG. 48. Alternatively, separate leads may be utilized for stimulation and blocking, and the pulse generator may be adapted for two or three leads, as was previously described in conjunction with FIG. 35A.

Therefore in the methods and systems of this disclosure, in one embodiment stimulation without block may be provided. Additionally, stimulation with selective block may be provided. Blocking of nerve impulses, unidirectional blocking, and selective blocking of nerve impulses is well known in the scientific literature. Some of the general literature is listed below and is incorporated herein by reference. (a) “Generation of unidirectionally propagating action potentials using a monopolar electrode cuff”, Annals of Biomedical Engineering, volume 14, pp. 437-450, By Ira J. Ungar et al. (b) “An asymmetric two electrode cuff for generation of unidirectionally propagated action potentials”, IEEE Transactions on Biomedical Engineering, volume BME-33, No. 6, June 1986, By James D. Sweeney, et al. (c) A spiral nerve cuff electrode for peripheral nerve stimulation, IEEE Transactions on Biomedical Engineering, volume 35, No. 11, November 1988, By Gregory G. Naples. et al. (d) “A nerve cuff technique for selective excitation of peripheral nerve trunk regions, IEEE Transactions on Biomedical Engineering, volume 37, No. 7, July 1990, By James D. Sweeney, et al. (e) “Generation of unidirectionally propagated action potentials in a peripheral nerve by brief stimuli”, Science, volume 206 pp.1311-1312, Dec. 14, 1979, By Van Den Honert et al. (f) “A technique for collision block of perpheral nerve: Frequency dependence” IEEE Transactions on Biomedical Engineering, MP-12, volume 28, pp. 379-382, 1981, By Van Den Honert et al. (g) “A nerve cuff design for the selective activation and blocking of myelinated nerve fibers” Ann. Conf. of the IEEE Engineering in Medicine and Biology Soc., volume 13, No. 2, p 906, 1991, By D. M Fitzpatrick et al. (h) “Orderly recruitment of motoneurons in an acute rabbit model”, “Ann. Conf. of the IEEE Engineering in Medicine and Biology Soc., volume 20, No. 5, page 2564, 1998, By N. J. M. Rijkhof, et al. (i) “Orderly stimulation of skeletal muscle motor units with tripolar nerve cuff electrode”, IEEE Transactions on Biomedical Engineering, volume 36, No. 8, pp. 836, 1989, By R. Bratta. (j) M. Devor, “Pain Networks”, Handbook of Brand Theory and Neural Networks, Ed. M. A. Arbib, MIT Press, page 698, 1998.

Blocking can be generally divided into 3 categories: (a) DC or anodal block, (b) Wedenski Block, and (c) Collision block. In anodal block there is a steady potential which is applied to the nerve causing a reversible and selective block. In Wedenski Block the nerve is stimulated at a high rate causing the rapid depletion of the neurotransmitter. In collision blocking, unidirectional action potentials are generated anti-dromically. The maximal frequency for complete block is the reciprocal of the refractory period plus the transit time, i.e. typically less than a few hundred hertz. The use of any of these blocking techniques can be applied in the practice of this disclosure, and all are considered within the scope of this disclosure.

Since one of the objects of this disclosure is to decease side effects such as hoarseness in the throat, or any cardiac side effects, blocking electrodes may be strategically placed at the relevant branches of vagus nerve.

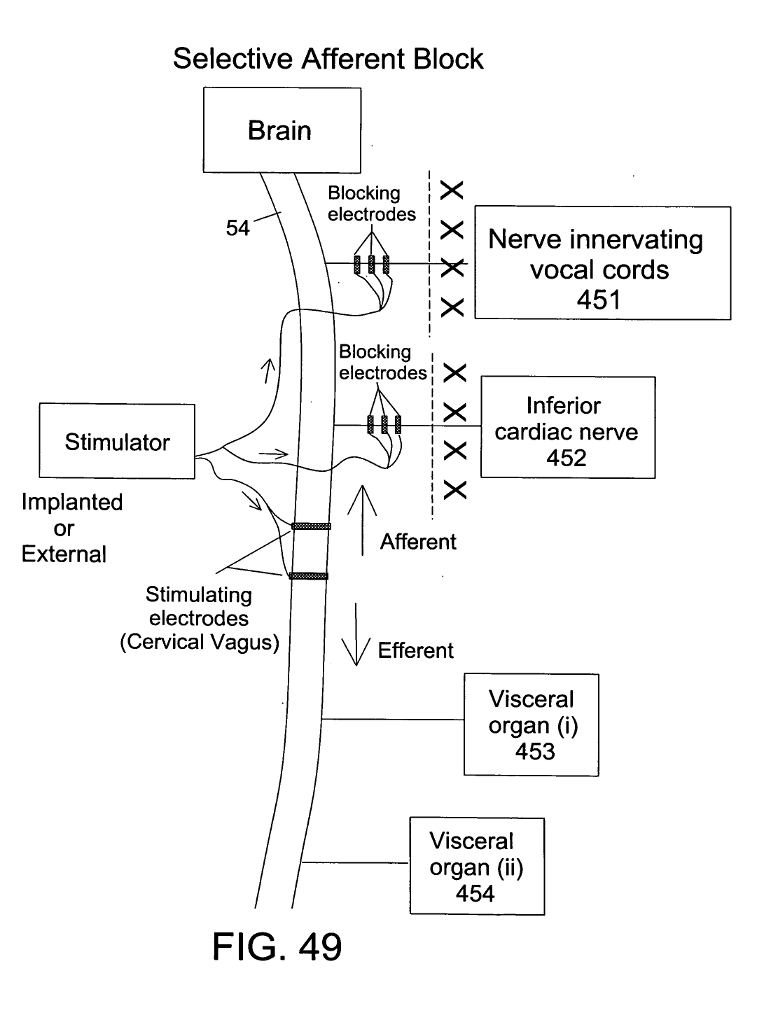

As shown in conjunction with FIG. 49, the stimulating electrodes are placed on cervical vagus, and the blocking electrodes are placed on a branch to vocal cords 451. With the blocking electrodes positioned between the vocal cords and the stimulating electrodes, and the controller supplying blocking pulses to the blocking electrode, the side effects pertaining to vocal response can be eliminated or significantly diminished. Advantageously, more aggressive therapy can be provided, leading to even better efficacy. Similarly, as also depicted in FIG. 49, the blocking electrode may be placed on the inferior cardiac nerve 452, whereby the blocking electrode would be positioned between the heart and stimulating electrode. Again, with the controller delivering blocking pulses to the blocking electrode, the cardiac side effects would be significantly diminished or virtually eliminated.

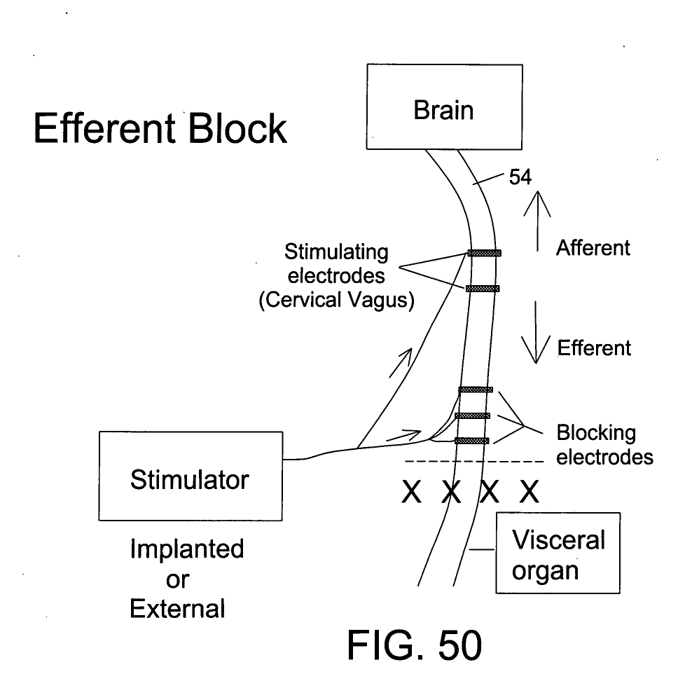

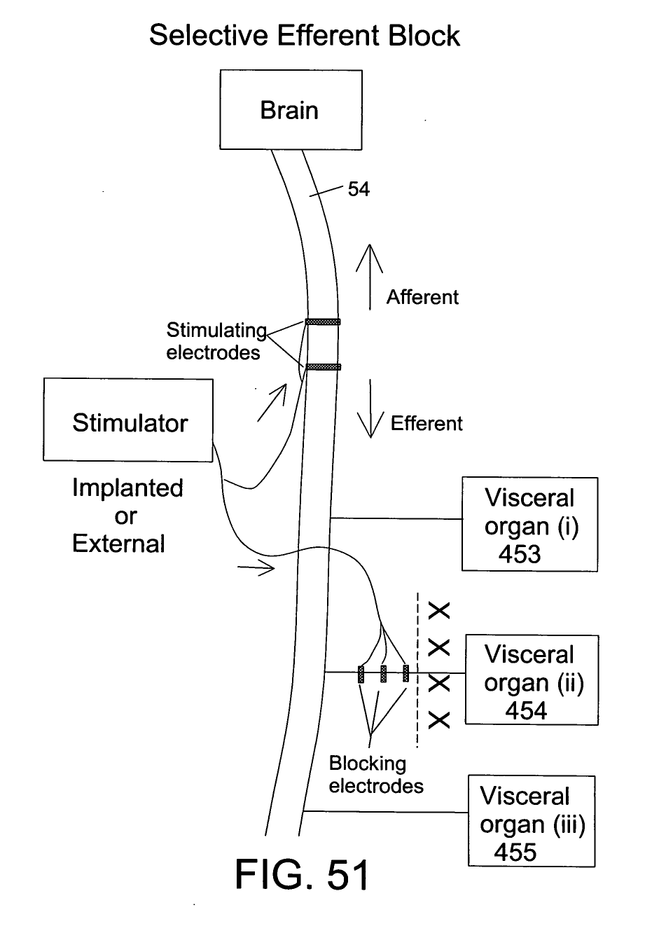

Shown in conjunction with FIG. 50 is simplified depiction of efferent block. This time with the blocking electrodes placed distal to the stimulating electrode(s), and the controller supplying blocking pulses to the blocking electrodes, the efferent pulses can be essentially blocked. Advantageously, the side effects related to cardiopulmonary system, gastrointestinal system and pancreobiliary system can be greatly diminished. It will be apparent to one skilled in the art that, as shown in conjunction with FIG. 51, selective efferent block can also be performed.

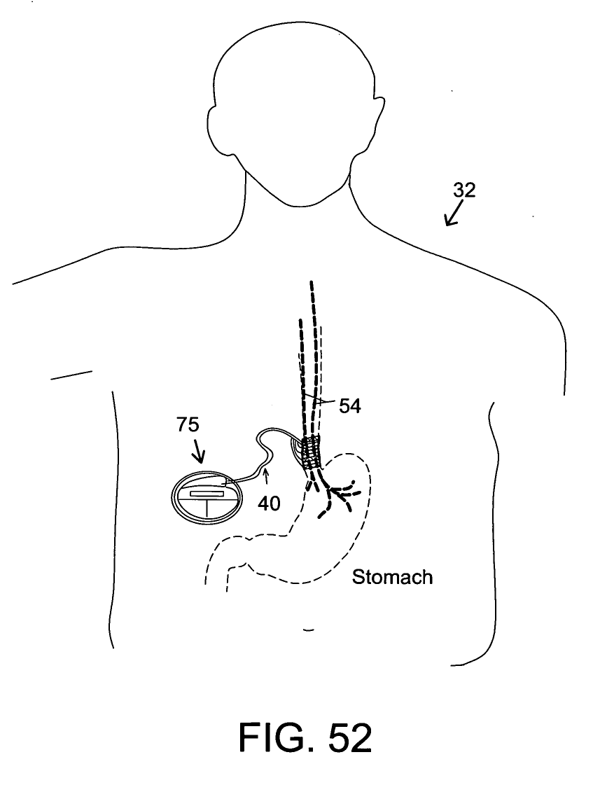

In one aspect of the disclosure, the pulsed electrical stimulation/blocking to the vagus nerve(s) may be provided anywhere along the length of the vagus nerve(s). As was shown earlier in conjunction with FIG. 20, the pulsed electrical stimulation/blocking may be at the cervical level for some applications. Alternatively, shown in conjunction with FIG. 52, the stimulation/blocking to the vagus nerve(s) may be around the diaphramatic level, either just above the diaphragm or below the diaphragm. Further, the stimulation may be unilateral or bilateral, i.e. stimulation is to one or both vagus nerves or its branches or parts thereof. Any combination of vagal nerve(s) stimulation/blocking, either unilateral or bilateral, anywhere along the length of the vagal nerve(s) is considered within the scope of this disclosure. Providing electrode configuration for stimulation and/or blocking at around the diaphragm level is particularly useful in providing therapy for obesity and other gastrointestinal (GI) disorders. In some applications, down regulating or blocking vagus nerve(s) at around the stomach level induces gastroporesis which gives a feeling of satiety, furthermore, down regulating the vagus nerve also decreases secretions from the pancreas, which also provides therapy for obesity.

### Programming

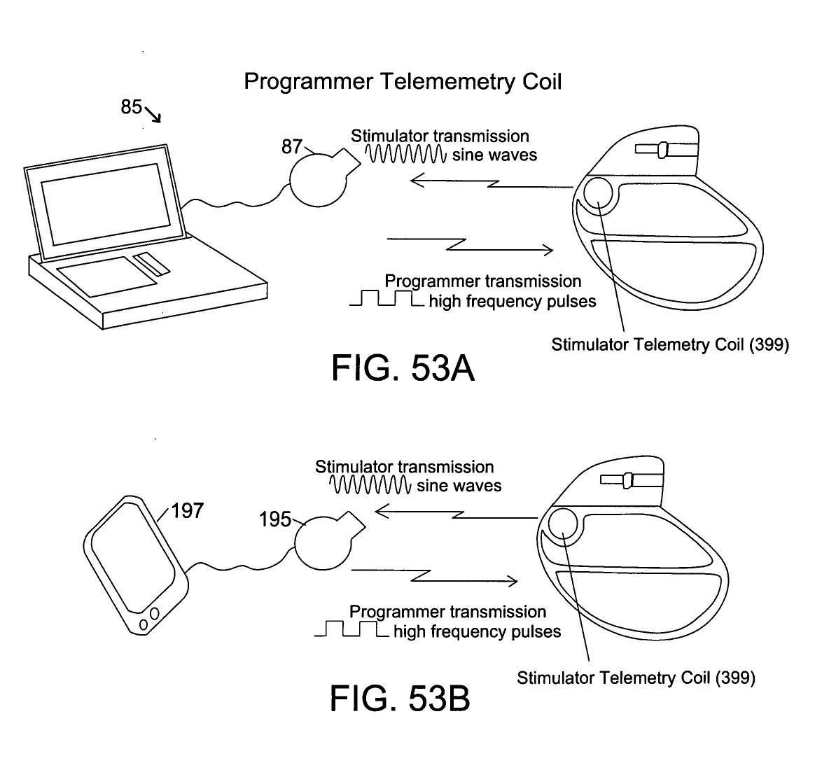

The programming of the implanted pulse generator (IPG) 391 is shown in conjunction with FIGS. 53A and 53B. With the magnetic Reed Switch 389 (FIG. 34) in the closed position, a coil in the head of the programmer 85, communicates with a telemetry coil 399 of the implanted pulse generator 391. Bi-directional inductive telemetry is used to exchange data with the implanted unit 391 by means of the external programming unit 85.

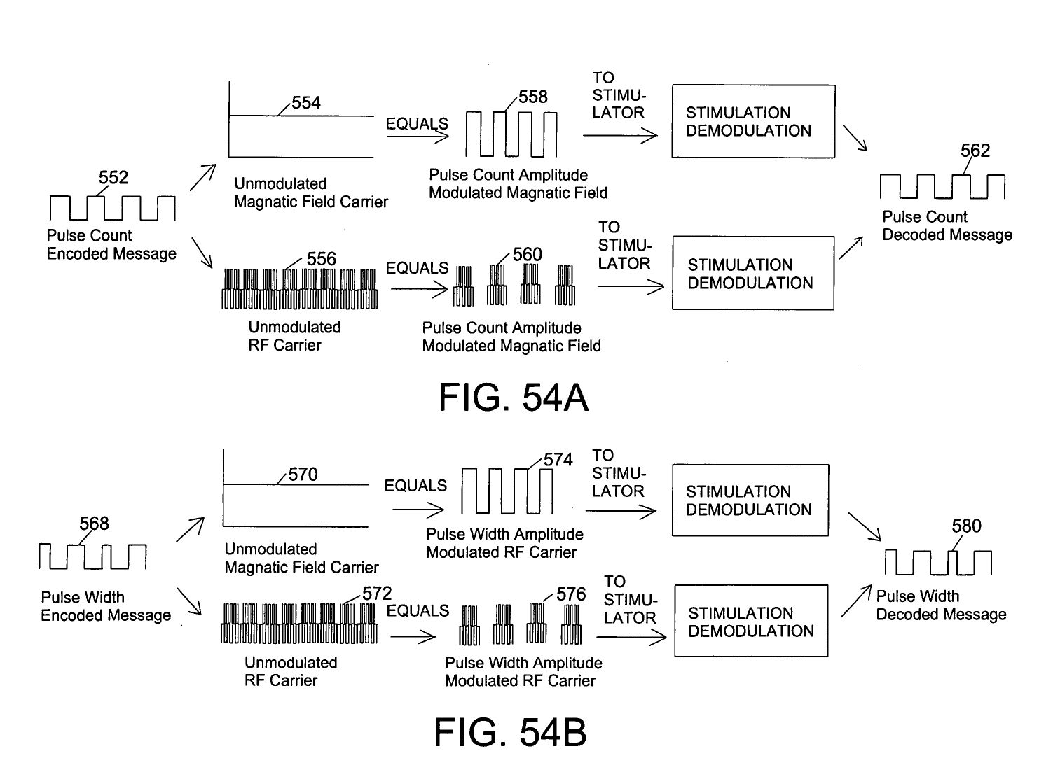

The transmission of programming information involves manipulation of the carrier signal in a manner that is recognizable by the pulse generator 391 as a valid set of instructions. The process of modulation serves as a means of encoding the programming instruction in a language that is interpretable by the implanted pulse generator 391. Modulation of signal amplitude, pulse width, and time between pulses are all used in the programming system, as will be appreciated by those skilled in the art. FIG. 54A shows an example of pulse count modulation, and FIG. 54B shows an example of pulse width modulation, that can be used for encoding.

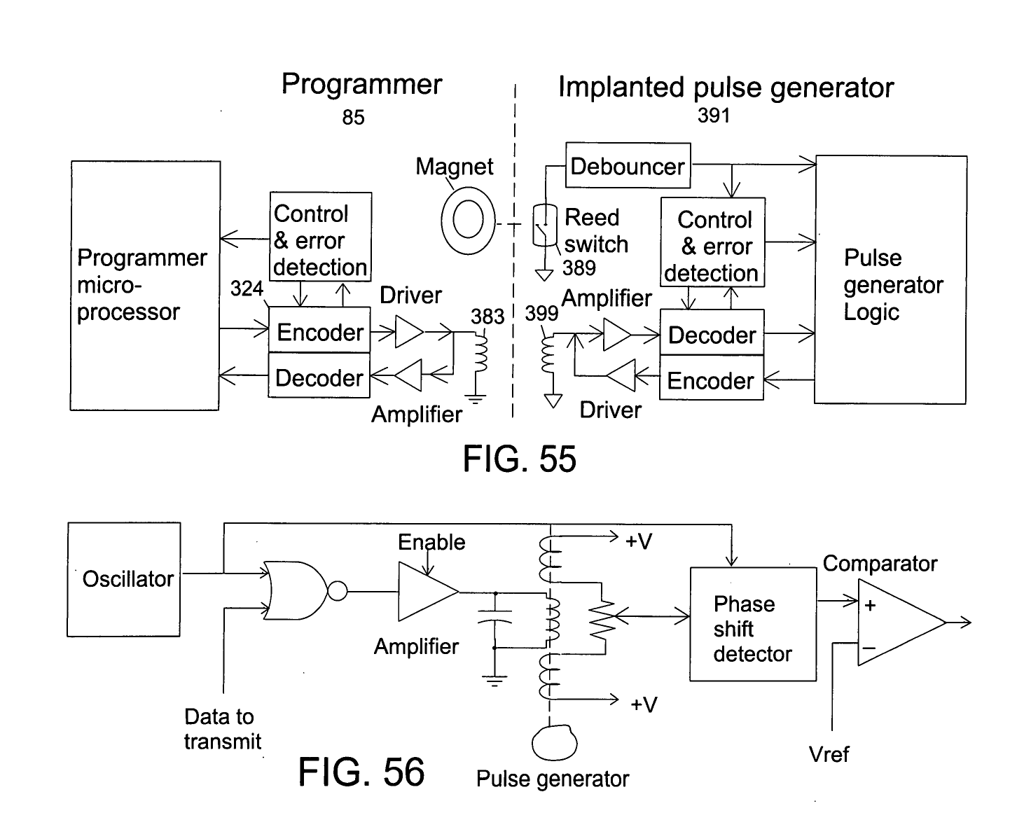

FIG. 55 shows a simplified overall block diagram of the implanted pulse generator (IPG) 391 programming and telemetry interface. The left half of FIG. 55 is programmer 85 which communicates programming and telemetry information with the IPG 391. The sections of the IPG 391 associated with programming and telemetry are shown on the right half of FIG. 55. In this case, the programming sequence is initiated by bringing a permanent magnet in the proximity of the IPG 391 which closes a reed switch 389 in the IPG 391. Information is then encoded into a special error-correcting pulse sequence and transmitted electromagnetically through a set of coils. The received message is decoded, checked for errors, and passed on to the unit's logic circuitry. The IPG 391 of this embodiment includes the capability of bidirectional communication.

The reed switch 389 is a magnetically-sensitive mechanical switch, which consists of two thin strips of metal (the “reed”) which are ferromagnetic. The reeds normally spring apart when no magnetic field is present. When a field is applied, the reeds come together to form a closed circuit because doing so creates a path of least reluctance. The programming head of the programmer contains a high-field-strength ceramic magnet.

When the switch closes, it activates the programming hardware, and initiates an interrupt of the IPG central processor. Closing the reed switch 389 also presents the logic used to encode and decode programming and telemetry signals. A nonmaskable interrupt (NMI) is sent to the IPG processor, which then executes special programming software. Since the NMI is an edge-triggered signal and the reed switch is vulnerable to mechanical bounce, a debouncing circuit is used to avoid multiple interrupts. The overall current consumption of the IPG increases during programming because of the debouncing circuit and other communication circuits.

A coil 399 is used as an antenna for both reception and transmission. Another set of coils 383 is placed in the programming head, a relatively small sized unit connected to the programmer 85. All coils are tuned to the same resonant frequency. The interface is half-duplex with one unit transmitting at a time.

Since the relative positions of the programming head 87 and IPG 391 determine the coupling of the coils, this embodiment utilizes a special circuit which has been devised to aid the positioning of the programming head, and is shown in FIG. 56. It operates on similar principles to the linear variable differential transformer. An oscillator tuned to the resonant frequency of the pacemaker coil 399 drives the center coil of a three-coil set in the programmer head. The phase difference between the original oscillator signal and the resulting signal from the two outer coils is measured using a phase shift detector. It is proportional to the distance between the implanted pulse generator and the programmer head. The phase shift, as a voltage, is compared to a reference voltage and is then used to control an indicator such as an LED. An enable signal allows switching the circuit on and off.

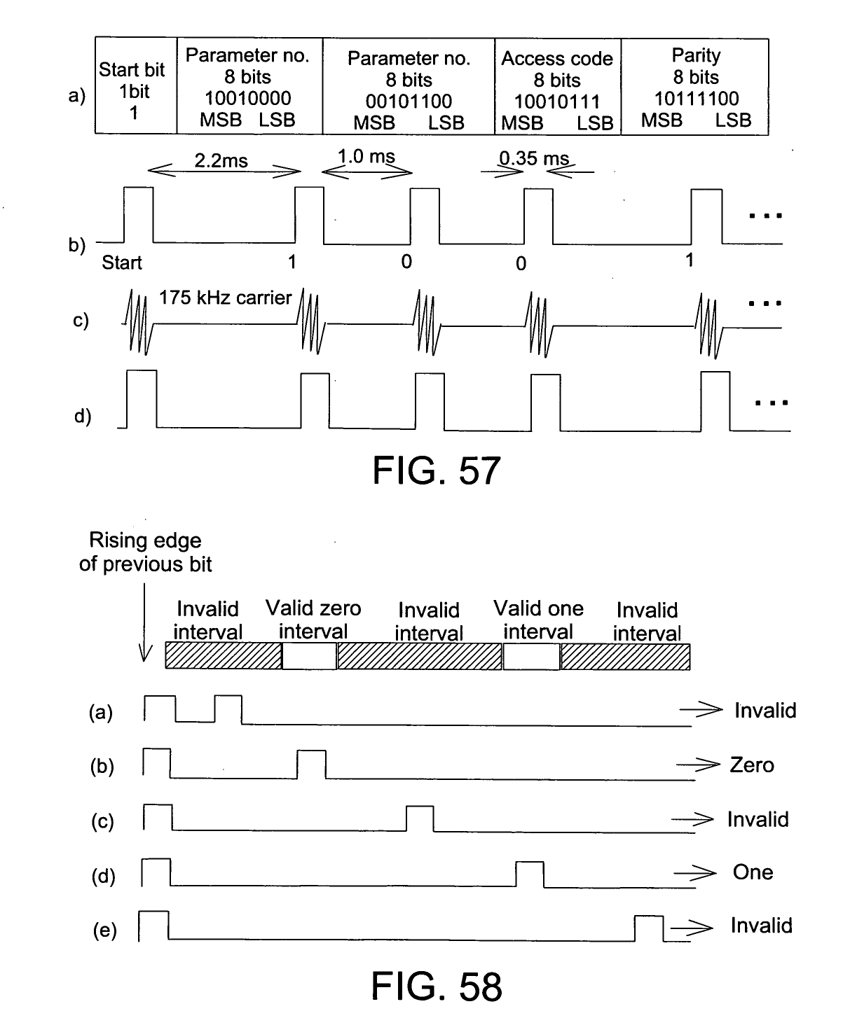

Actual programming is shown in conjunction with FIGS. 57 and 58. Programming and telemetry messages comprise many bits; however, the coil interface can only transmit one bit at a time. In addition, the signal is modulated to the resonant frequency of the coils, and must be transmitted in a relatively short period of time, and must provide detection of erroneous data.

A programming message is comprised of five parts FIG. 57(a). The start bit indicates the beginning of the message and is used to synchronize the timing of the rest of the message. The parameter number specifies which parameter (e.g., mode, pulse width, delay) is to be programmed. In the example, in FIG. 57(a) the number 10010000 specifies the pulse rate to be specified. The parameter value represents the value that the parameter should be set to. This value may be an index into a table of possible values; for example, the value 00101100 represents a pulse stimulus rate of 80 pulses/min. The access code is a fixed number based on the stimulus generator model which must be matched exactly for the message to succeed. It acts as a security mechanism against use of the wrong programmer, errors in the message, or spurious programming from environmental noise. It can also potentially allow more than one programmable implant in the patient. Finally, the parity field is the bitwise exclusive-OR of the parameter number and value fields. It is one of several error-detection mechanisms.

All of the bits are then encoded as a sequence of pulses of 0.35-ms duration FIG. 57(b). The start bit is a single pulse. The remaining bits are delayed from their previous bit according to their bit value. If the bit is a zero, the delay is short (1.0); if it is a one, the delay is long (2.2 ms). This technique of pulse position coding, makes detection of errors easier.

The serial pulse sequence is then amplitude modulated for transmission FIG. 57(c). The carrier frequency is the resonant frequency of the coils. This signal is transmitted from one set of coils to the other and then demodulated back into a pulse sequence FIG. 57(d).

FIG. 58 shows how each bit of the pulse sequence is decoded from the demodulated signal. As soon as each bit is received, a timer begins timing the delay to the next pulse. If the pulse occurs within a specific early interval, it is counted as a zero bit (FIG. 58(b)). If it otherwise occurs with a later interval, it is considered to be a one bit (FIG. 58(d)). Pulses that come too early, too late, or between the two intervals are considered to be errors and the entire message is discarded (FIG. 58(a, c, e)). Each bit begins the timing of the bit that follows it. The start bit is used only to time the first bit.

Telemetry data may be either analog or digital. Digital signals are first converted into a serial bit stream using an encoding such as shown in FIG. 58(b). The serial stream or the analog data is then frequency modulated for transmission.

An advantage of this and other encodings is that they provide multiple forms of error detection. The coils and receiver circuitry are tuned to the modulation frequency, eliminating noise at other frequencies. Pulse-position coding can detect errors by accepting pulses only within narrowly-intervals. The access code acts as a security key to prevent programming by spurious noise or other equipment. Finally, the parity field and other checksums provides a final verification that the message is valid. At any time, if an error is detected, the entire message is discarded.

Another more sophisticated type of pulse position modulation may be used to increase the bit transmission rate. In this, the position of a pulse within a frame is encoded into one of a finite number of values, e.g. 16. A special synchronizing bit is transmitted to signal the start of the frame. Typically, the frame contains a code which specifies the type or data contained in the remainder of the frame.

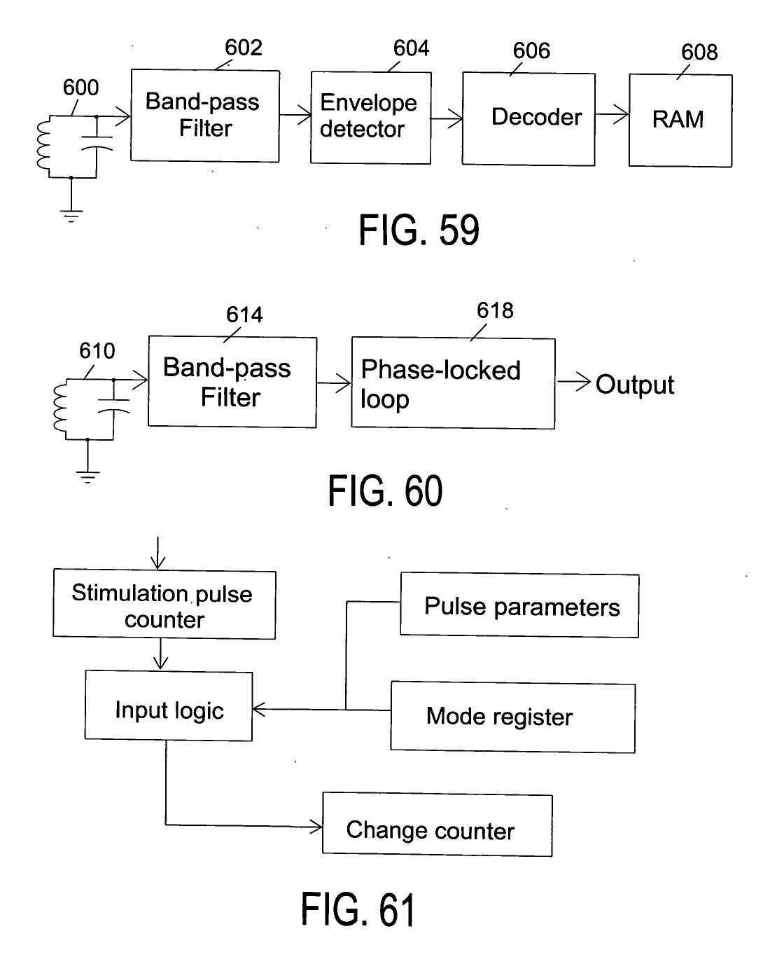

FIG. 59 shows a diagram of receiving and decoding circuitry for programming data. The IPG coil, in parallel with capacitor creates a tuned circuit for receiving data. The signal is band-pass filtered 602 and envelope detected 604 to create the pulsed signal in FIG. 57(d). After decoding, the parameter value is placed in a RAM at the location specified by the parameter number. The IPG can have two copies of the RAM—a permanent set and a temporary set—which makes it easy for the physician to set the IPG to a temporary configuration and later reprogram it back to the usual settings.

FIG. 60 shows the basic circuit used to receive telemetry data. Again, a coil and capacitor create a resonant circuit tuned to the carrier frequency. The signal is further band-pass filtered 614 and then frequency-demodulated using a phase-locked loop 618.

This embodiment also comprises an optional battery status test circuit. Shown in conjunction with FIG. 61, the charge delivered by the battery is estimated by keeping track of the number of pulses delivered by the IPG 391. An internal charge counter is updated during each test mode to read the total charge delivered. This information about battery status is read from the IPG 391 via telemetry.

### Wireless Telemetry with Programmer

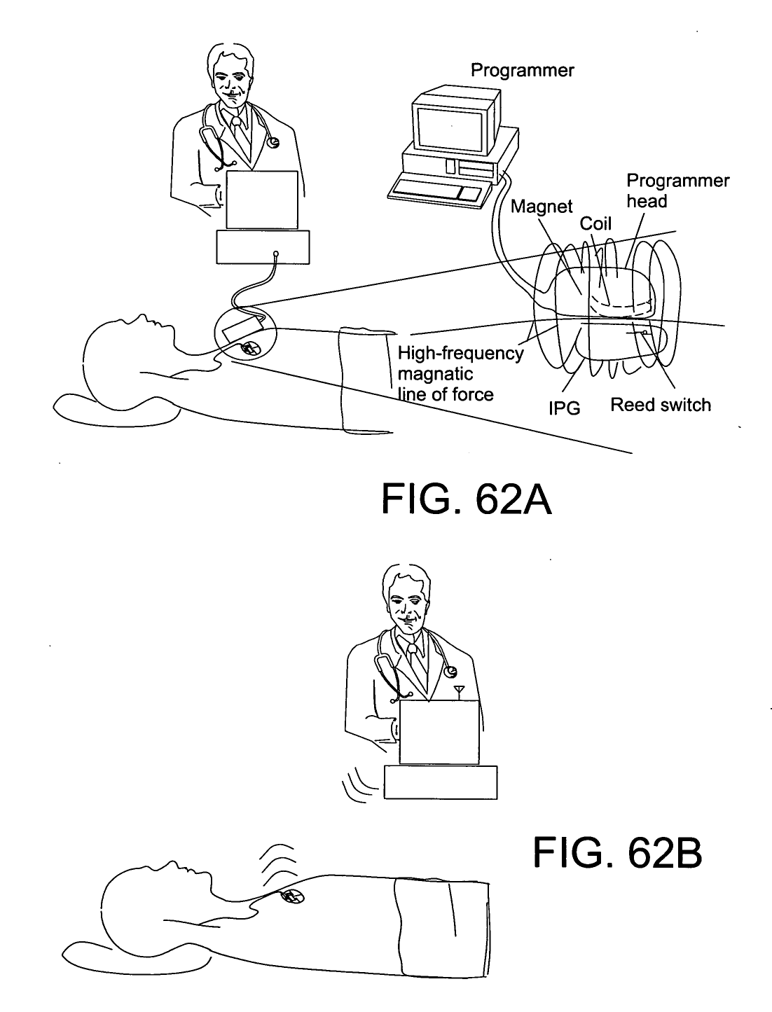

The communication between the external programmer and the implanted pulse generator may be via magnetic inductive-coupling as depicted in FIG. 62A, or may be utilizing wireless communication, as depicted in FIG. 62B, using Medical Implant Communications Service (MICS) or other UHF band of frequency.

The Federal Communications Commission (FCC) has assigned the MICS band of frequencies in the 402-405 MHz range for implanted medical devices (IMD). In the FCC's MICS standard and European Standards for ultra-low-power active medical implants (ULP-AMIS), the devices are optimized for operation in the 402-405 MHz frequency band. The 402-405 MHz frequency band is available for MICS operations on a shared, secondary basis. Currently, the MICS standard allows 10 channels of 300 kHz each and limits the output power to 25 microwatts. The FCC has proposed to revise its nomenclature and designate the entire 401-406 MHz band as MedRadio service. For the purposes of this disclosure, any mention of the MICS band will include the entire 401-406 MHz band which will be the MedRadio service.

For using such wireless telemetry, implant antenna design poses several technical hurdles stemming primarily from the small antenna size and location within the body, with the poor transmission medium—muscle, fat and skin—through which the signal must pass. The body has a high electric conductivity that results in large path loss in the transmission of energy from the implant to free air space.

The 401-406 MHz frequency band has several advantages such as: a) a low power transmitter and antenna designed specifically for the MICS band can be made small enough and still have excellent performance over a six-foot transmission range; b) the frequency range does not pose an interference risk with other radios operating in the same band; and c) the frequencies in the MICS band have propagation characteristics that are conducive to the transmission of radio signals within the human body.

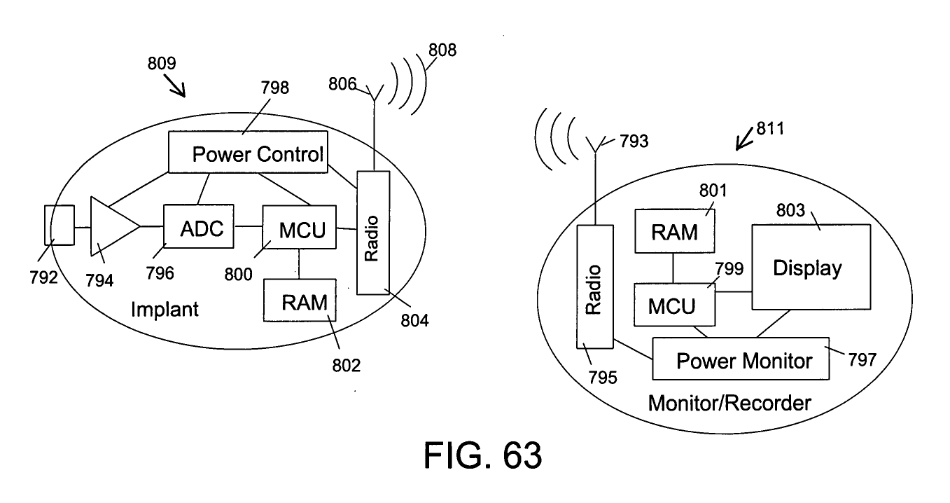

FIG. 63 generally illustrates the main components of a typical MICS RF system. The sensor block 792 acquires analog signals which are amplified and filtered 794 before being digitized via an A/D converter 796. The signals are processed via a microcontroller 800 (in conjunction with on-board memory 802), modulated on to a carrier signal, conditioned, and are transmitted 808 via an antenna 806 in the implanted device 809. An antenna 793 in an external device 811 which is physically within 6 feet of the implanted device 809, picks up the signals 808, after some conditioning the signals are demodulated, processed using the microcontroller 799 and displayed 803.

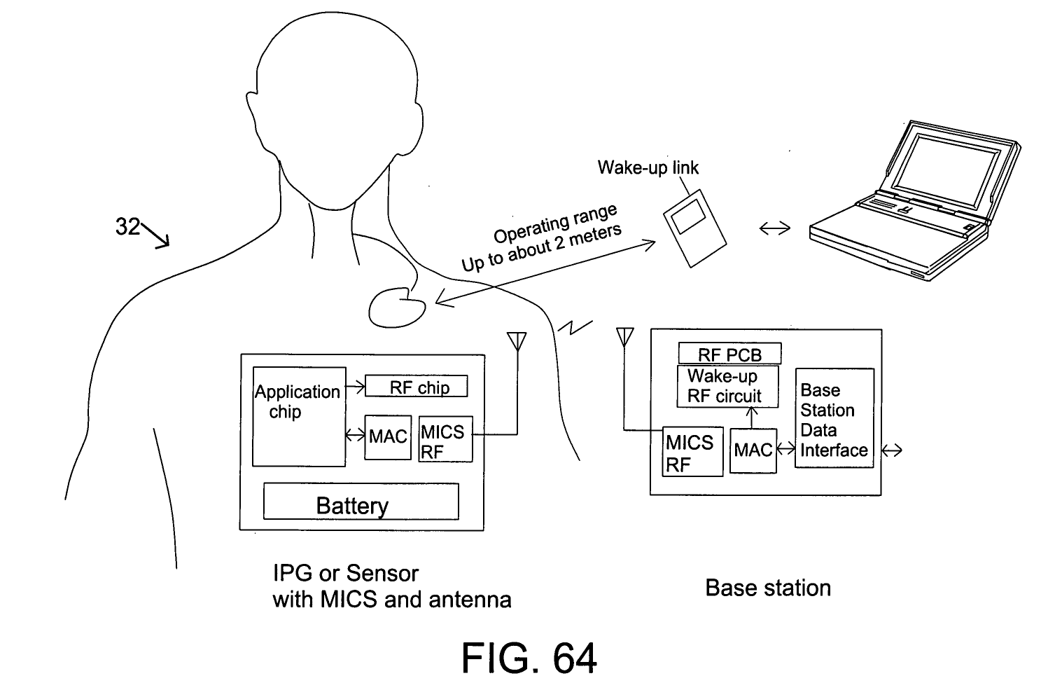

FIG. 64 depicts the application of transceiver IC specifically designed for implanted medical devices operating in the 401-406 MHz MICS band, for the current application of vagal nerve modulation. The concepts of duty cycling, ultra-low-power circuit design, and high integration levels are incorporated, with specific attention paid to the special needs of an implanted pulse generator (IPG) system.

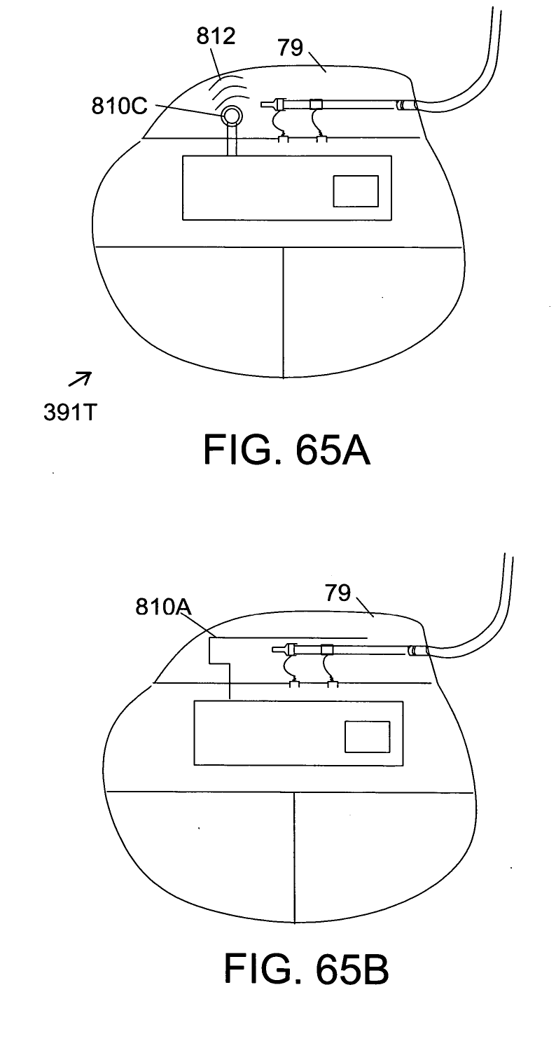

In this disclosure, in embodiments where wireless telemetry is utilized, the telemetry coil (antenna) of the implanted pulse generator 391T is externalized outside the titanium case into the header region 79. This is done utilizing standard glass-metal, or ceramic-metal feed-through as is known in the art. This is depicted in conjunction with FIGS. 65A and 65B, where the telemetry antenna 810C is shown above the titanium case, in the header region 79 which is generally made of a clear solid encapsulated material such as an epoxy, thermal setting polymer such as silicone or the like. The difference between FIGS. 65A and 65B is the shape of the antenna 810C in FIG. 65A and antenna 810A in FIG. 65B

Currently, magnetic inductively-coupled systems support one-way communications at data rates of about 50 kbits/s and at a range of only a few inches. Advantageously, an RF link can achieve up to 250 kbits/s at a six-foot range, but the penalty comes in power consumption. For an implant device with a desired battery life of five to seven years, every joule of energy must be carefully conserved.

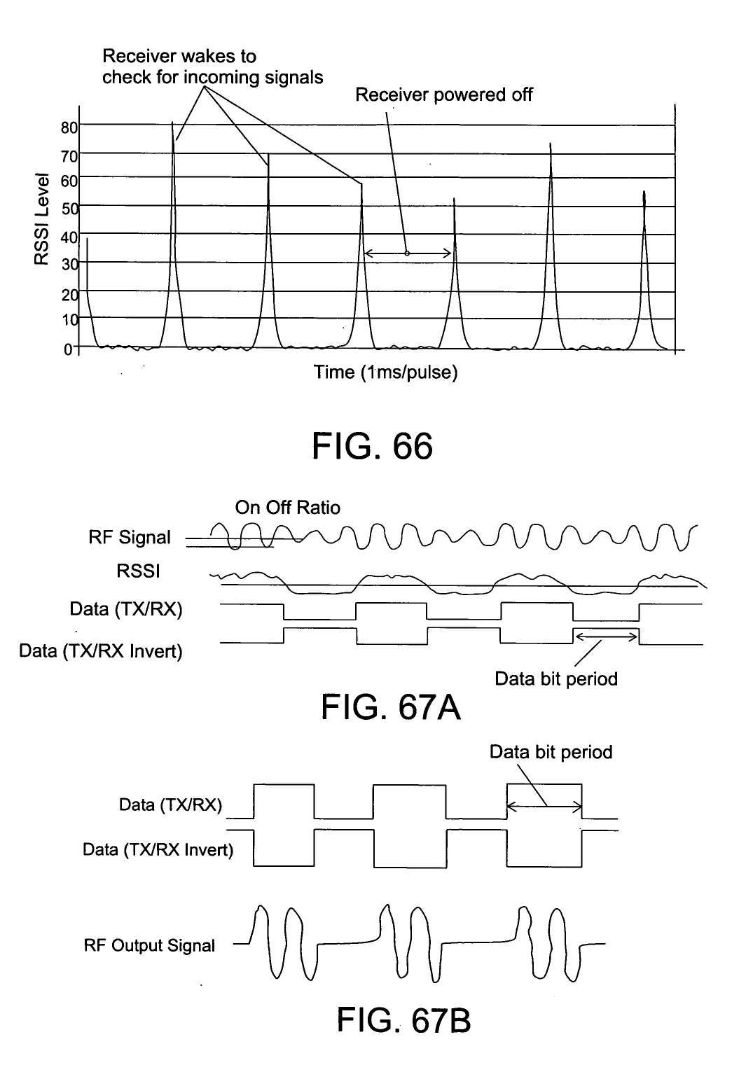

Chip-based RF transceivers employ several different techniques to keep the power down. One technique is to keep the power-hungry transmitter circuits powered off when not required. An additional technique is to periodically power-down the receiver circuits to conserve additional power, as is shown in FIG. 66. The entire duty cycle can be completed in less than 100 micro-seconds, which makes it possible to achieve very low average power consumption while monitoring the MICS channel for transmitted messages. The transceiver is “off” most of the time, meaning the off-state current and the current required to periodically look for a communication device is extremely low (less than 1 μA). In both cases, low power (less than 6 mA) for transmit and receive is also required. Both techniques require a rapid-start oscillator that can wake up the receiver (if needed) and the transmitter in an extremely short period of time.

Either amplitude or frequency modulation schemes may be used. The amplitude-shift keying/on-off keying (ASK/OOK) and frequency-shift keying (FSK) methods are popular modulation schemes in narrow MICS channel application. FIG. 67A depicts amplitude-shift keying, and FIG. 67B depicts on-off keying, both of which may be used. Twin-independent receive channels helps improve the reliability of MICS transmissions so that power is not wasted with retransmissions.

More sophisticated transceivers also contain baseband clock and data recovery (CDR) circuits that post-process the demodulated incoming data stream to produce both a sampled data bit stream and a clock signal. That process helps improve transmission reliability by synchronizing the data processing clock with the incoming data.

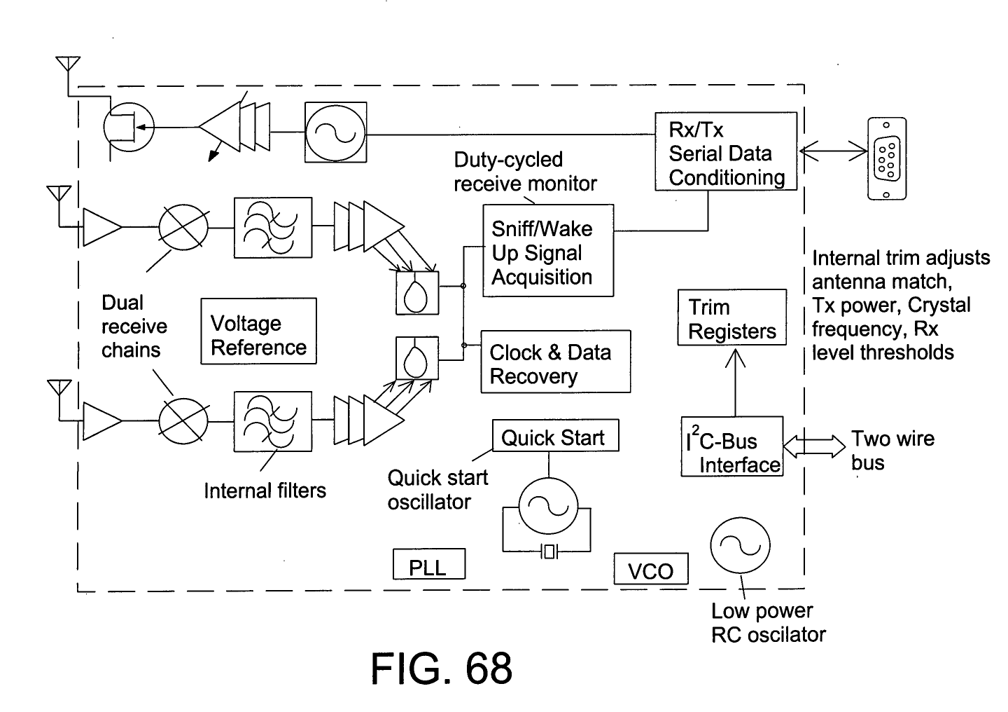

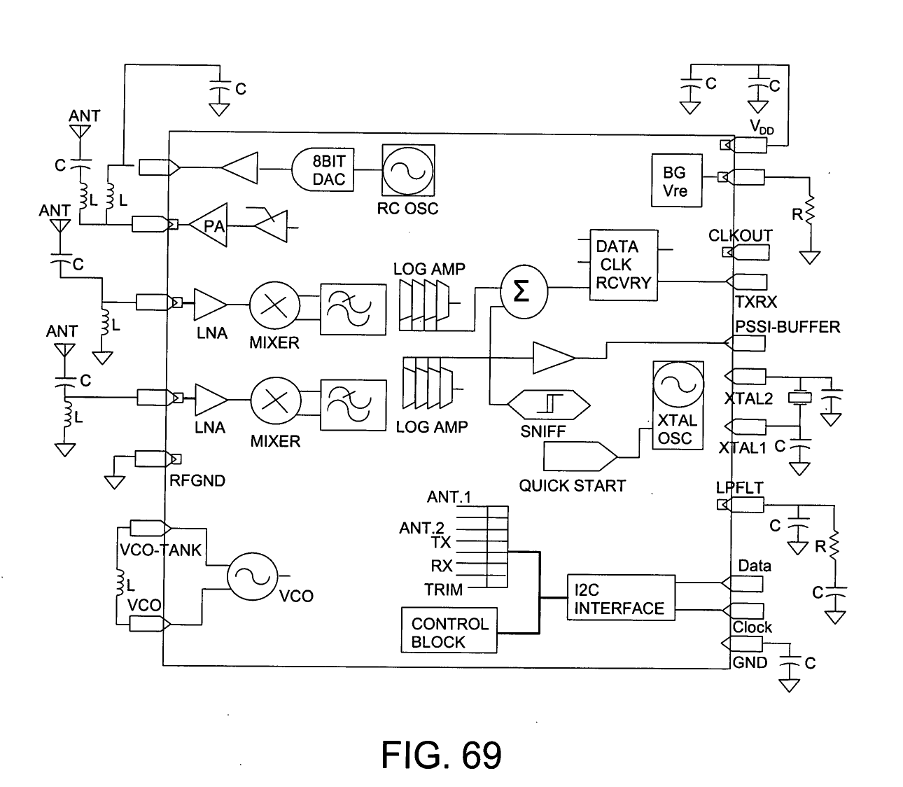

A block diagram of a low-power radio transceiver is shown in FIG. 68. In one embodiment, a crystal controlled, micropower transceiver chip such as available from AMI Semiconductor (Pocatello, Id.) AMIS-52100 (shown in FIG. 69), which is specifically designed for such applications may be used. A detailed circuitry of this chip is shown in FIG. 69. This chip is responsible for generating the RF carrier during transmissions and for amplifying, receiving, and detecting (converting to a logic level) the received RF signals. One skilled in the art will readily appreciate that other functionally equivalent chips such as available from Zarlink Semiconductor, may also be used. For example, a recently introduced chip from Zarlink, the ZL70100 allows data transmission rates of 500 Kbits/S over a typical 2 meter range.

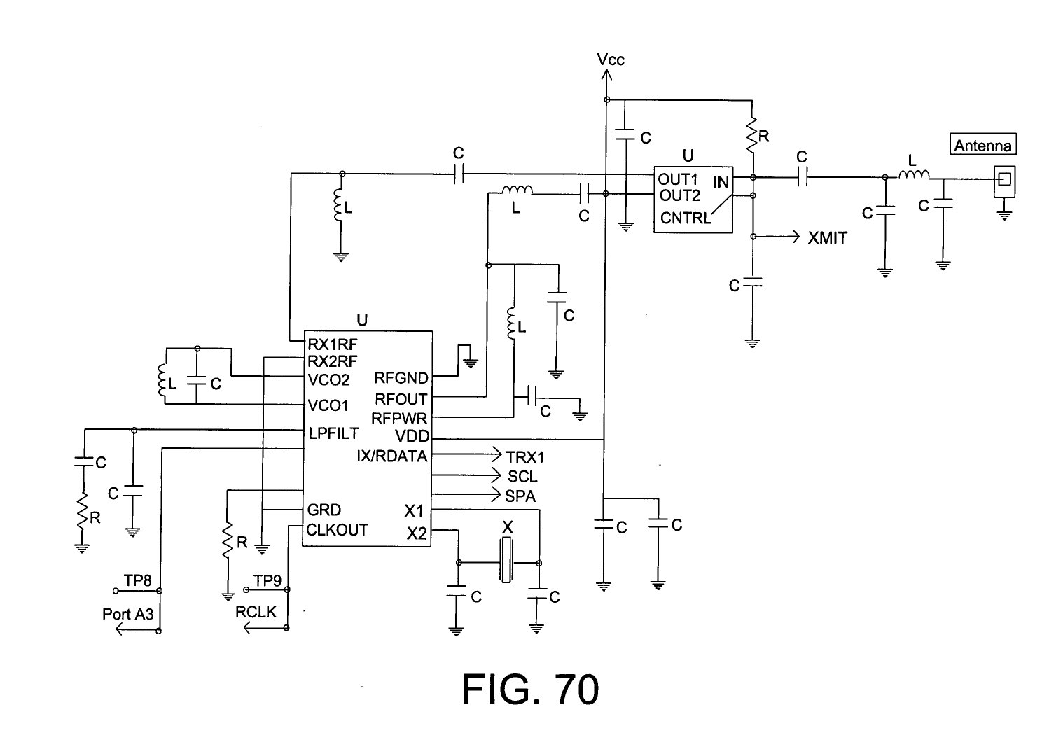

An example of a prior art workable telemetry circuitry is shown in FIG. 70

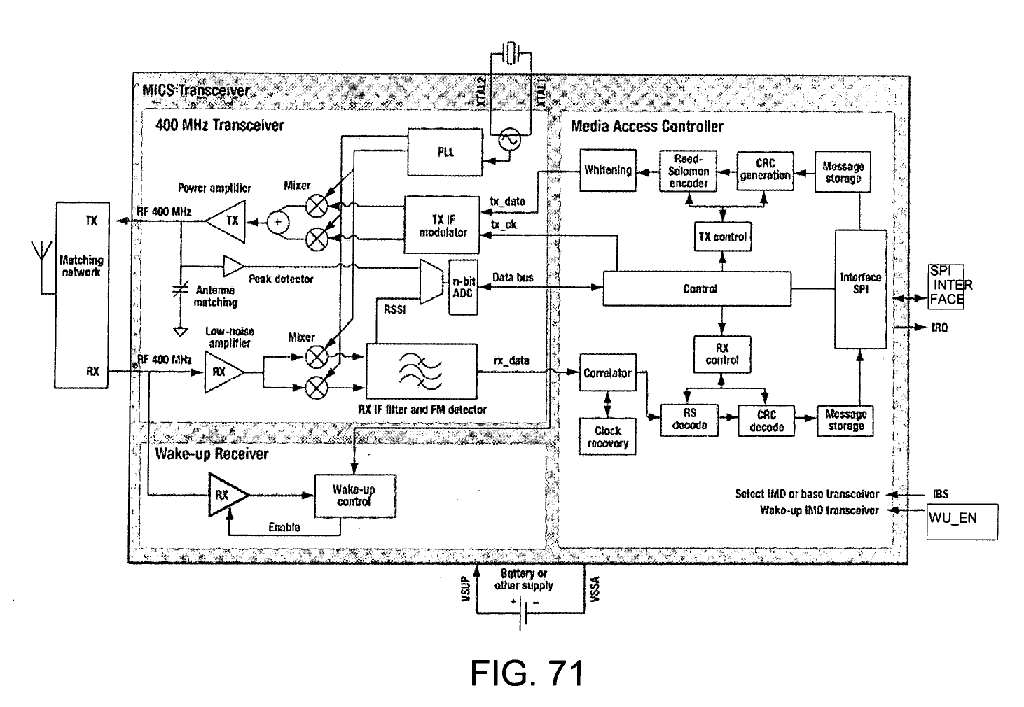

As was previously mentioned one of several modulation schemes may be used. Quadature amplitude modulation and Nyquist-filtered M-ary (or multiple) phase modulation both offer good bandwidth efficiencies. However, constant-envelope signals (i.e., FSK) are advantageous because they result in replaced requirements on the linearity of the system. Of the available modulation schemes, FSK modulation is another scheme which has been found to provide a good compromise between data rate, complexity, and requirements on linearity. FSK allows for a high-data-rate, low-power receiver. FIG. 71 shows a block diagram of a Ultra-Low-Power MICS Transceiver architecture which uses frequency-shift-keyed (FSK) modulation with varying frequency deviations.

Since most implant applications use the MICS RF link infrequently (because of their overriding need to conserve battery power), in very-low-power applications, the transceiver spends most of the time asleep in a very-low-current state and periodically sniffs for a wake-up signal. This sniffing operation has to be frequent enough to provide reasonable start-up latency, and because it will occur regularly it should consume a very low current. It should also be immune to noise sources that invoke an erroneous start-up. In this situation, an on-off keyed (OOK) modulation scheme is used because the OOK scheme removes the need for a local oscillator and synthesizer in the receiver, both of which require time and power to start up.

The wake-up system-shown in FIG. 71 uses an ultra-low-power RF receiver to read OOK transmitted data. The receiver's main function is to detect the incoming signal from the programmer and then to activate the rest of the chip. The example shown may also be started directly by pin control, which allows either an external programmer to initiate communication or the implant itself to send an emergency communication.

### Patient Hand-held Programmer

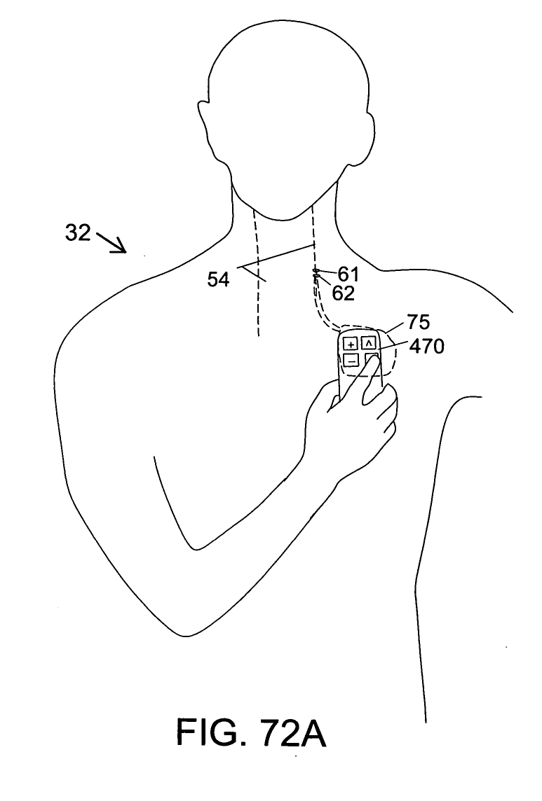

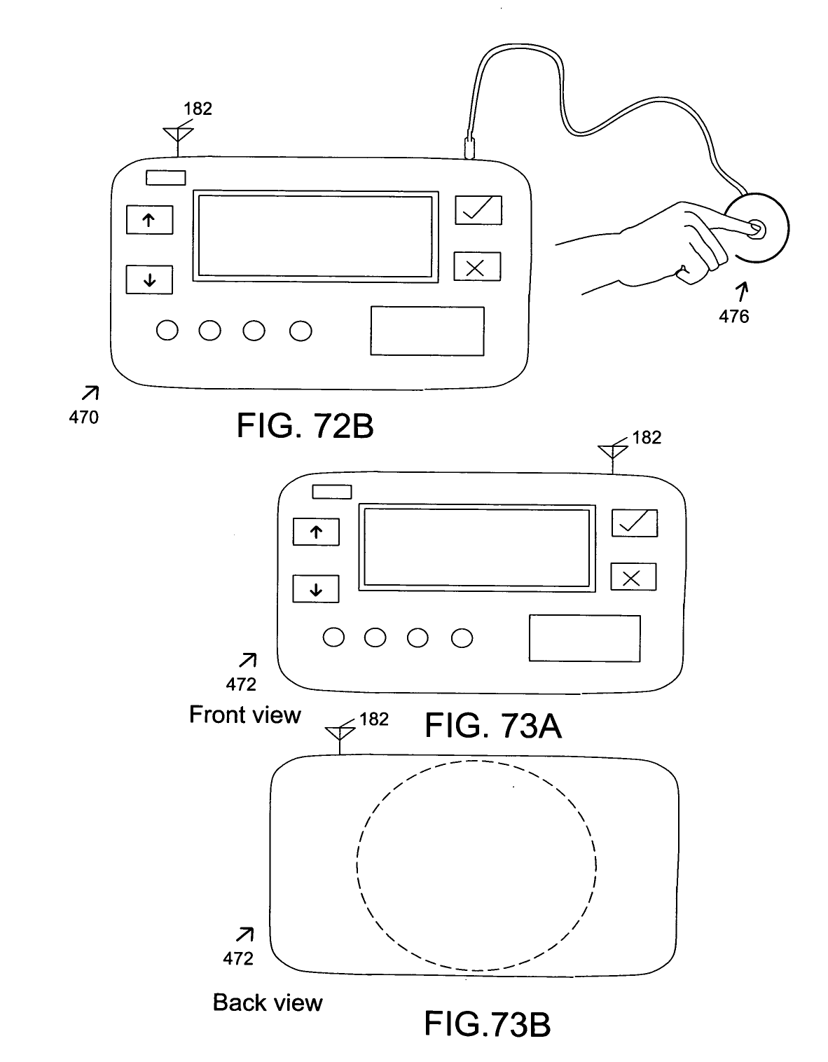

In one aspect of the disclosure, the patient 32 is provided with a separate hand-held programmer (with limited functionality) and can adjust the level of stimulation with a patient programmer 470, within predefined limits established by a physician or clinician. The patient programmer 470 may also comprise any number of predetermine/pre-packaged programs stored in the memory. The patient programmer 470 is shown in conjunction with FIGS. 72A and 72B. The patient programmer of this embodiment may also be used to activate specific predetermine/pre-packaged program from a limited selection specified by a physician or clinician, or to adjust certain variable parameters. The patient programmer 470 differs from a general programmer 85 (as used by a physician) in that the patient programmer has limited functionality, is extremely simple to use and is specifically adapted to be rugged.

As discussed in the previous section, the patient programmer may utilize wireless telemetry for communication with the implanted stimulator. In other embodiments, the communication between a patient programmer 470 and an implanted stimulator 75 may be magnetic inductively coupled.

As shown in conjunction with FIG. 72A, in one embodiment the patient places the patient programmer 470 over the implanted device 75 and simply presses and holds the increase or decrease buttons for a pre-determined period of time. In an one embodiment, as shown in conjunction with FIG. 72B, an optional external coil (antenna) 476 is also provided with the patient programmer 470. The optional external coil 476 is attached to the patient programmer 470 via a flimsy tubing which covers the conductor. In this embodiment the patient programmer 470 may remain on a belt, or in a pocket, and the patient positions the external coil on the implanted device 75 with one hand and presses the increase or decrease buttons for a pre-determined time period.

In one aspect, the patient programmer 470 may also be networked via an antenna 182, as shown in conjunction with FIGS. 73A and 73B, and is described later.

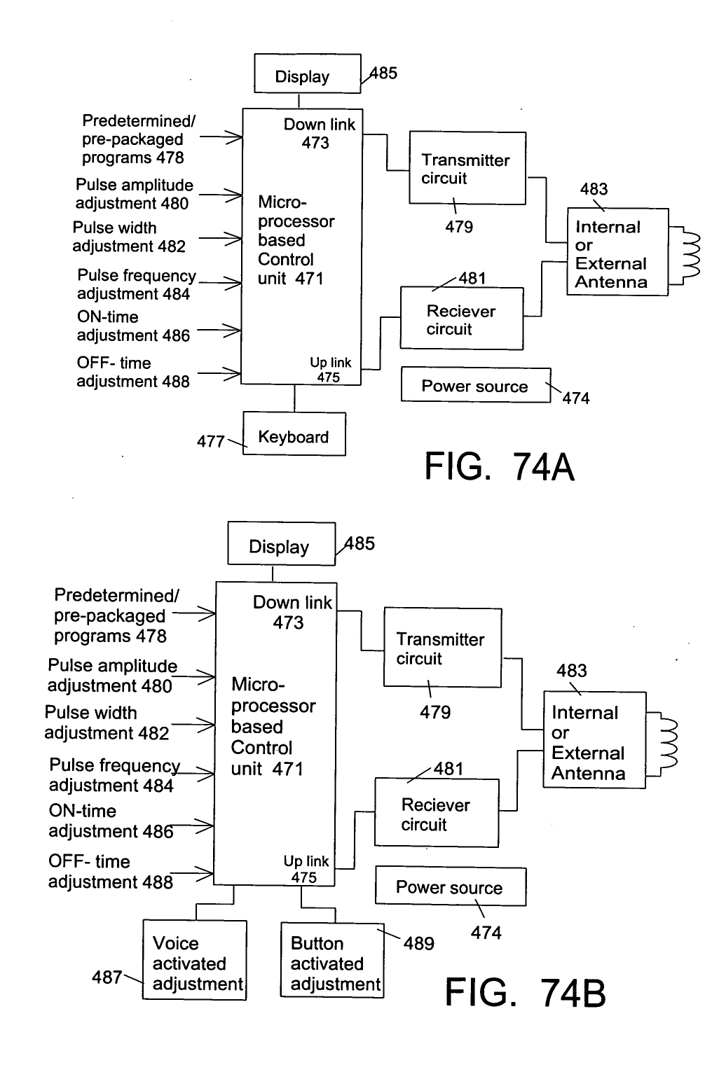

Shown in conjunction with FIG. 74A is an overall block diagram of the patient programmer 470 of one embodiment. The microprocessor based control unit 471 is configured to change between different predetermined/pre-packaged programs 478, or to increase or decrease individual parameters of a program that has been programmed or activated. These individual parameters include pulse amplitude 480, pulse width 482, pulse frequency 484, ON-time 486, and OFF-time 488. Other parameter adjustments may also be incorporated into the patient programmer 470.

The patient programmer 470 comprises a radio-frequency (RF) transmitter 479 and receiver 481 which communicate with the corresponding RF transmitter and receiver in the IPG as was shown previously. The electrical signals are transmitted or received via an antenna 483, which communicates with the implanted device. Within the implantable device, the transmitter and receiver utilize a wire coil as an antenna for receiving down-link telemetry signals and for radiating RF signals for up-plink telemetry. In order to communicate digital data using RF telemetry, a digital encoding scheme such as described in U.S. Pat. No. 5,127,404 to Wyborny et al. may be used. Other digital encoding schemes well known in the art may also be used.

In one embodiment, as shown in conjunction with FIG. 74B, parameter adjustments may be performed either utilizing buttons 489 or via voice-activated commands 487. Software and hardware to configure voice activated commands is well known in the art.

### Combination Implantable Device Comprising Both a Stimulus-receiver and a Programmable Implantable Pulse Generator (IPG)

In one embodiment, the implantable device may comprise both a stimulus-receiver and a programmable implantable pulse generator (IPG) in one device. Another embodiment of a similar device is disclosed in applicant's co-pending application Ser. No. 10/436,017 which is incorporated herein by reference. This embodiment optionally comprises predetermined/pre-packaged programs. Examples of several stimulation states were given in the previous sections, under “Programmer-less Implantable Pulse Generator (IPG)” and “Programmable Implantable Pulse Generator”. These predetermined/pre-packaged programs comprise unique combinations of pulse amplitude, pulse width, pulse frequency, ON-time and OFF-time. One predetermined/pre-packaged program is ON/OFF program.

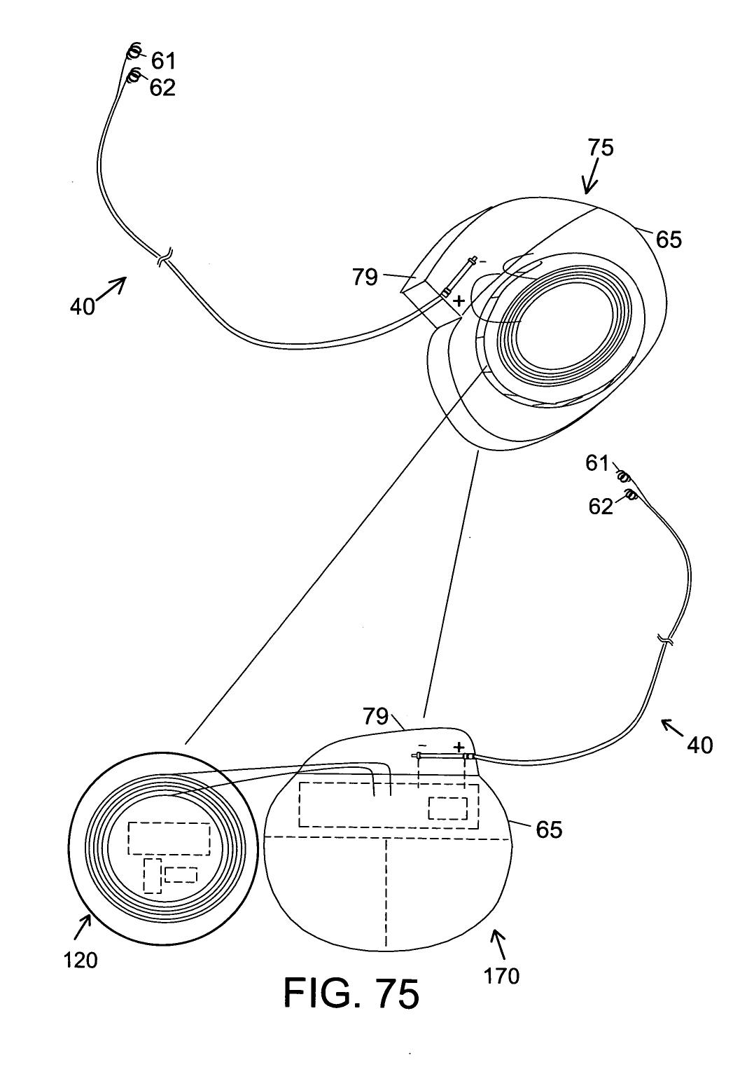

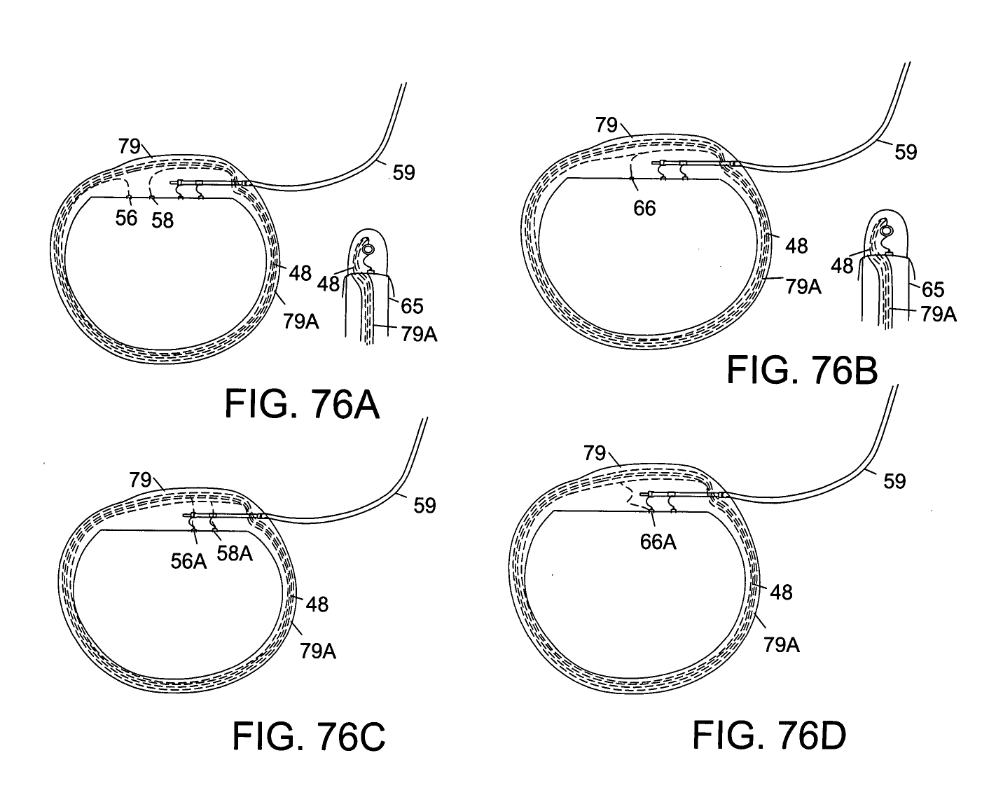

FIG. 75 shows a close up view of the packaging of the implanted stimulator 75 of this embodiment, showing the two subassemblies 120, 170. The two subassemblies are the stimulus-receiver module 120 and the battery operated pulse generator module 170. The electrical components of the stimulus-receiver module 120 may be substantially in the titanium case along with other circuitry, except for a coil. The coil may be outside the titanium case as shown in FIG. 75, or the coil 48C may be externalized at the header portion 79 of the implanted device, and may be wrapped around the titanium can. In this case, the coil is encased in the same material as the header 79, as shown in FIGS. 76A-76D. FIG. 76A depicts a bipolar configuration with two separate feed-throughs, 56, 58. FIG. 76B depicts a unipolar configuration with one separate feed-through 66. FIG. 76C, and 76D depict the same configuration except the feed-throughs are common with the feed-throughs 66A for the lead.

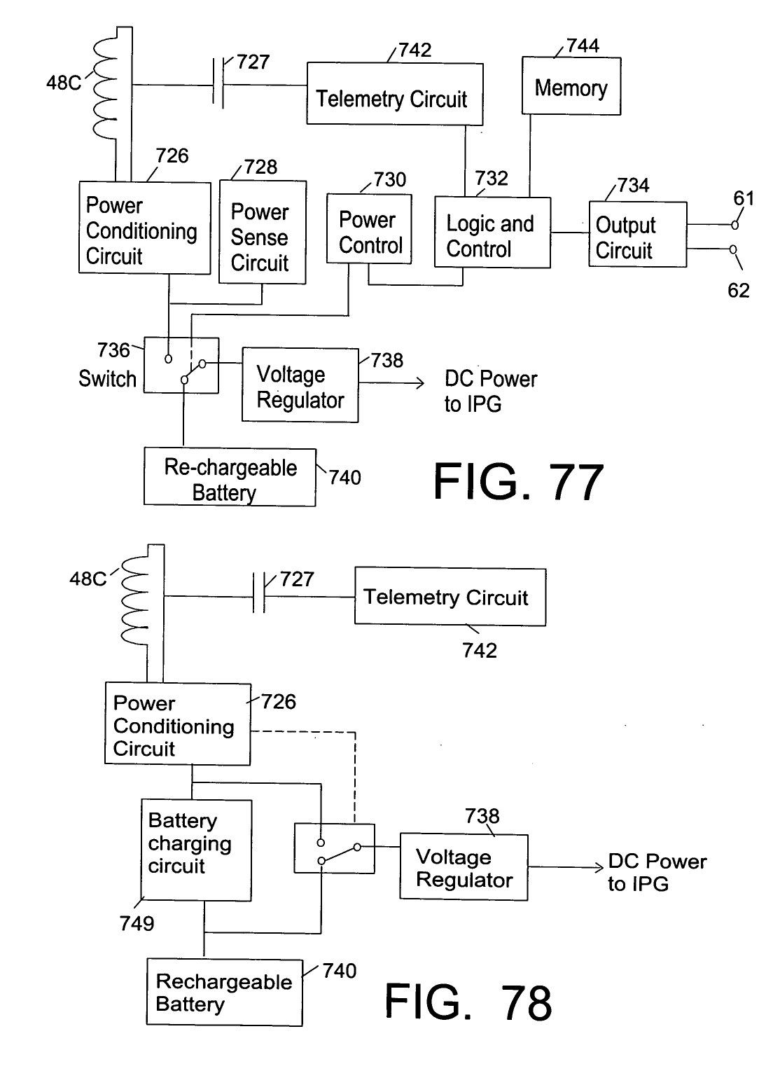

FIG. 77 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 77, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored changeable parameters. Using input for the telemetry circuit 742 and power control 730, this section controls the output circuit 734 that generates the output pulses.

It will be clear to one skilled in the art that this embodiment of the invention can also be practiced with a rechargeable battery. This version is shown in conjunction with FIG. 78. The circuitry in the two versions are similar except for the battery charging circuitry 749. This circuit is energized when external power is available. It senses the charge state of the battery and provides appropriate charge current to safely recharge the battery without overcharging.

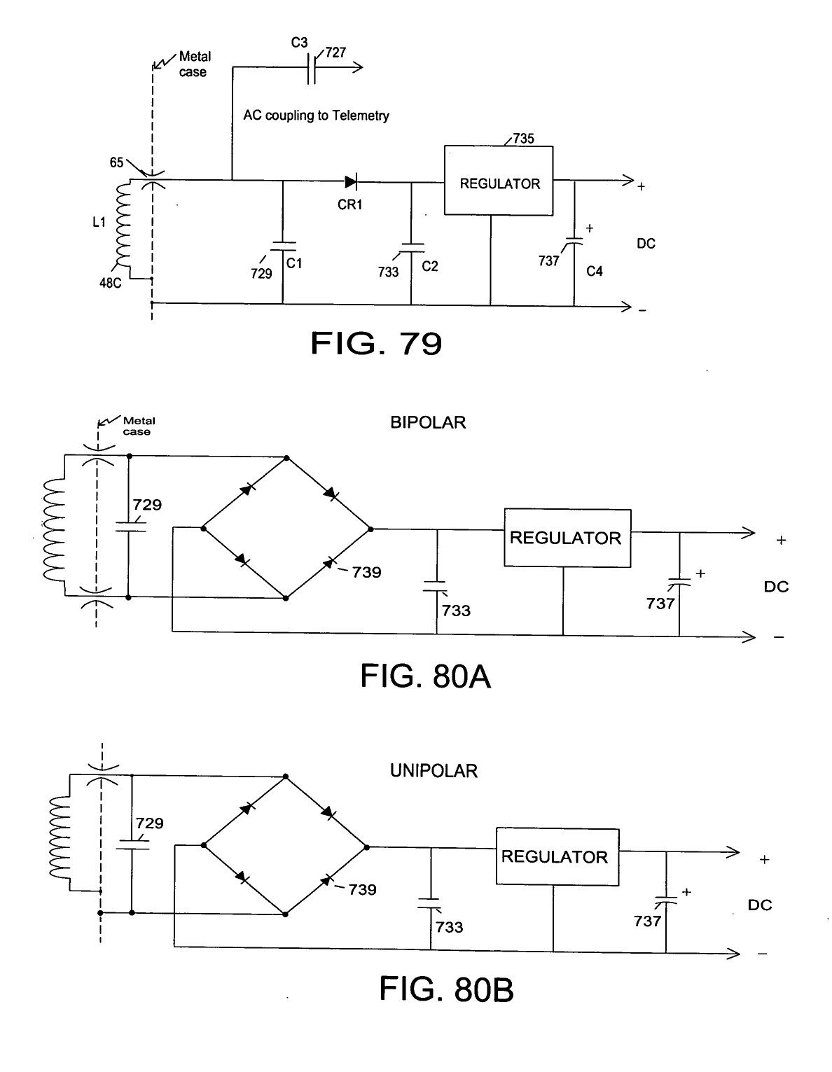

The stimulus-receiver portion of the circuitry is shown in conjunction with FIG. 79. Capacitor C1 (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 79, a capacitor C3 (727) couples signals for forward and back telemetry.

FIGS. 80A and 80B show alternate connection of the receiving coil. In FIG. 80A, each end of the coil is connected to the circuit through a hermetic feedthrough filter. In this instance, the DC output is floating with respect to the IPG's case. In FIG. 80B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliability of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case a return for the pulse current.

In the unipolar configuration, advantageously a bigger tissue area is stimulated since the difference between the tip (cathode) and case (anode) is larger. Stimulation using both configuration is considered within the scope of this invention.

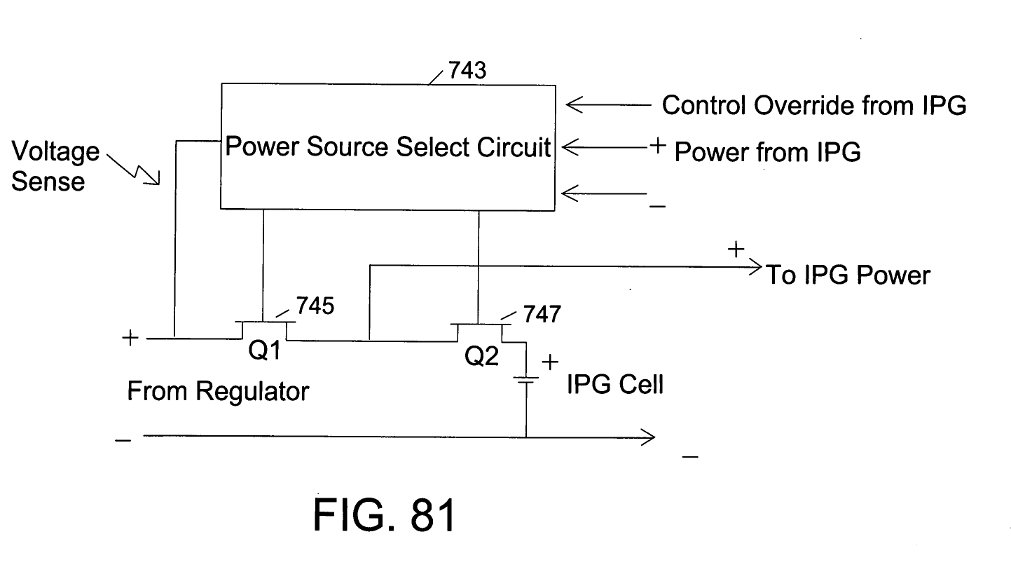

The power source select circuit is highlighted in conjunction with FIG. 81. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

### Implantable Pulse Generator (IPG) Comprising a Rechargable Battery

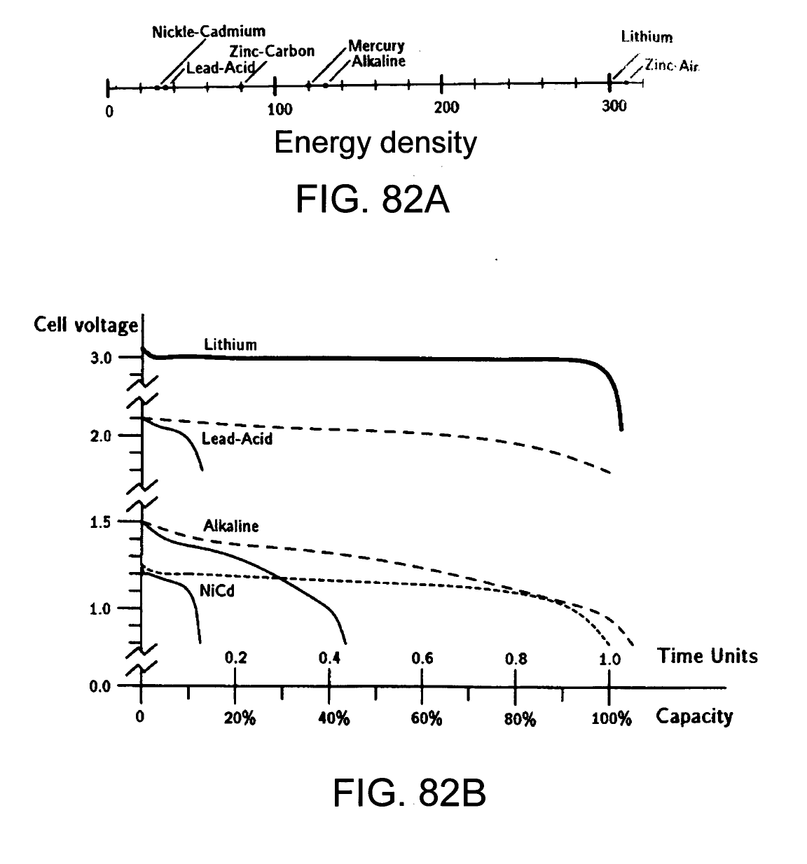

In one embodiment, an implantable pulse generator with rechargeable power source can be used. Because of the rapidity of the pulses required for modulating nerve tissue 54 with stimulating and/or blocking pulses, there is a real need for power sources that will provide an acceptable service life under conditions of continuous delivery of high frequency pulses. FIG. 82A shows a graph of the energy density of several commonly used battery technologies. Lithium batteries have by far the highest energy density of commonly available batteries. Also, a lithium battery maintains a nearly constant voltage during discharge. This is shown in conjunction with FIG. 82B, which is normalized to the performance of the lithium battery. Lithium-ion batteries also have a long cycle life, and no memory effect. However, Lithium-ion batteries are not as tolerant to overcharging and overdischarging. One of the most recent development in rechargable battery technology is the Lithium-ion polymer battery. Recently the major battery manufacturers (Sony, Panasonic, Sanyo) have announced plans for Lithium-ion polymer battery production.

This embodiment also comprises predetermined/pre-packaged programs. Examples of several stimulation states were given in the previous sections, under “Programmer-less Implantable Pulse Generator (IPG)” and “Programmable Implantable Pulse Generator”. These pre-packaged/pre-determined programs comprise unique combinations of pulse amplitude, pulse width, pulse frequency, ON-time and OFF-time. Additionally, predetermined programs comprising blocking pulses may also be stored in the memory of the pulse generator.

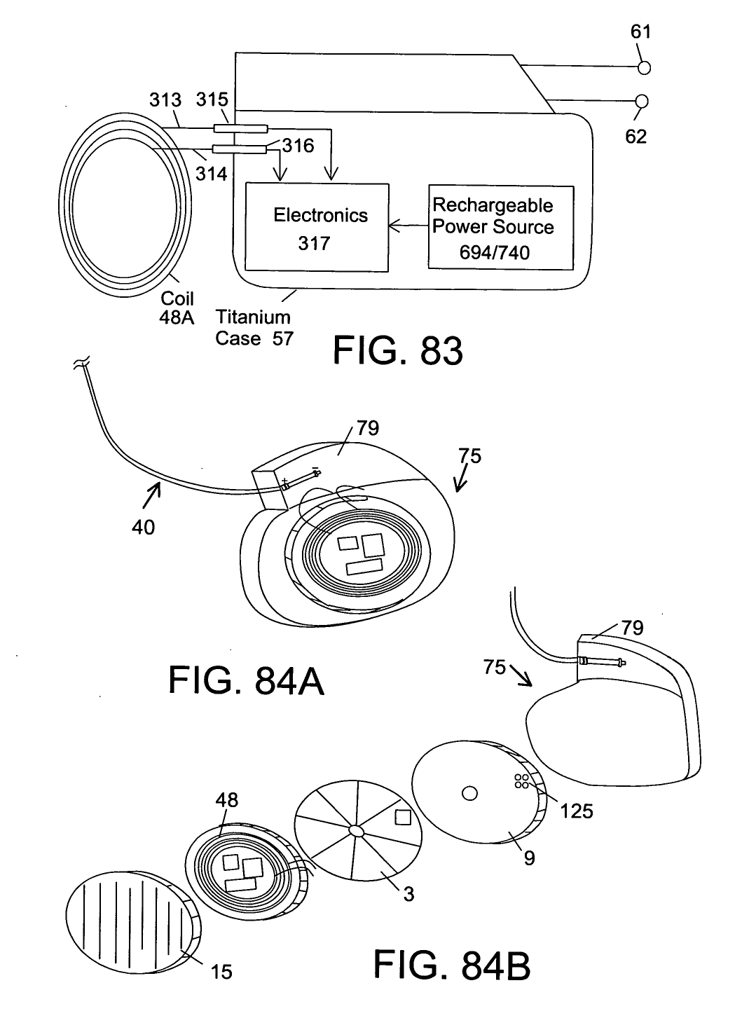

As shown in conjunction with FIG. 83, the coil is externalized from the titanium case 57. The RF pulses transmitted via coil 46 and received via subcutaneous coil 48A are rectified via a diode bridge. These DC pulses are processed and the resulting current applied to recharge the battery 6941740 in the implanted pulse generator. In one embodiment the coil 48C may be externalized at the header portion 79 of the implanted device, and may be wrapped around the titanium can, as was previously shown in FIGS. 76A-D.

In one embodiment, the coil may also be positioned on the titanium case as shown in conjunction with FIGS. 84A and 84B. FIG. 84A shows a diagram of the finished implantable stimulator 391R of one embodiment. FIG. 84B shows the pulse generator with some of the components used in assembly in an exploded view. These components include a coil cover 15, the secondary coil 48 and associated components, a magnetic shield 7, and a coil assembly carrier 19. The coil assembly carrier 9 has at least one positioning detail 125 located between the coil assembly and the feed through for positioning the electrical connection. The positioning detail 125 secures the electrical connection.

One skilled in the art will readily appreciate that in one embodiment, the recharge coil may be placed external to the titanium case and on the case with a magnetic shield between the coil and the titanium case. This is shown in conjunction with FIG. 83. In one embodiment, the recharge coil may be placed outside the titanium case, and around the case (FIGS. 76A-76D). In this embodiment, a magnetic shield is generally not required. Alternatively, in one embodiment the recharge coil may be place inside the titanium case. If the recharge coil is placed inside the titanium case, the thickness of the titanium case is carefully chosen such that there is a balance between the greater power absorption and shielding effects, to the low to medium frequency magnetic field used to transcutaneously recharge the Lithium Ion battery. In this embodiment, preferably low frequency (e.g., 30 KHz to 300 KHz) RF magnetic field are used.

In one embodiment, the recharge coil may be inside the titanium case. In this embodiment, the recharge coil, which desirably comprises a multi-turn, fine copper wire coil near the inside perimeter of the implantable stimulator. Preferably, the recharge coil includes a predetermined construction, e.g., desirably 250 to 350 turns, and more desirably 300 turns of four strands of #40 enameled magnetic wire, or the like. The maximizing of the coil's diameter and reduction of its effective RF resistance allows necessary power transfer at typical implant depth of about one centimeter.

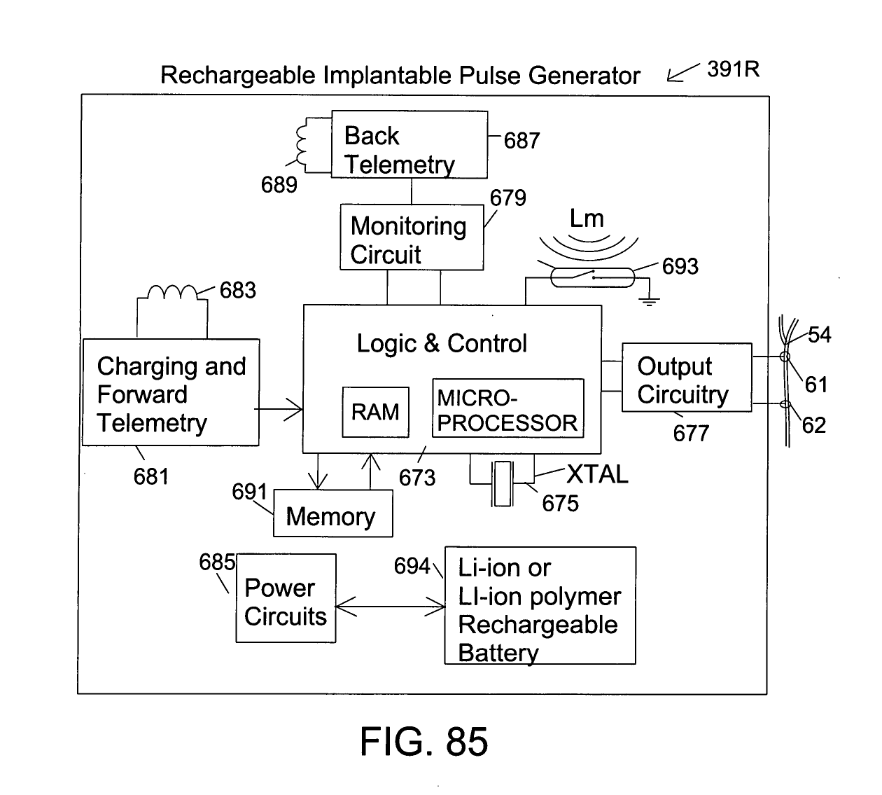

A schematic diagram of the implanted pulse generator (IPG 391R), with re-chargeable battery 694, is shown in conjunction with FIG. 85. The IPG 391R includes logic and control circuitry 673 connected to memory circuitry 691. The operating program and stimulation parameters are typically stored within the memory 691 via forward telemetry. Stimulation pulses are provided to the nerve tissue 54 via output circuitry 677 controlled by the microcontroller.

The operating power for the IPG 391R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

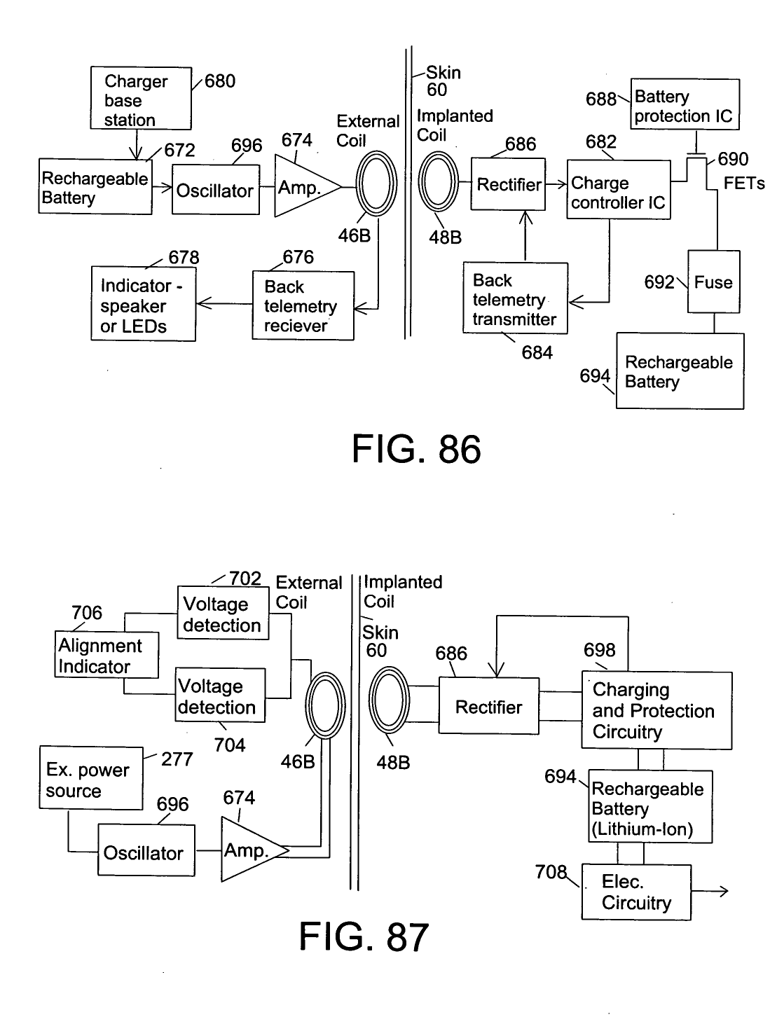

Shown in conjunction with FIG. 86 are the recharging elements of this embodiment. The re-charging system uses a portable external charger to couple energy into the power source of the IPG 391R. The DC-to-AC conversion circuitry 696 of the recharger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 86, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

A simplified block diagram of charge completion and misalignment detection circuitry is shown in conjunction with FIG. 87. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage Vₛ sensed by voltage detector 704 is at a minimum level because maximum energy transfer is taking place. If and when the coils 46B and 48B become misaligned, then less than a maximum energy transfer occurs, and the voltage Vₛ sensed by detection circuit 704 increases significantly. If the voltage Vₛ reaches a predetermined level, alignment indicator 706 is activated via an audible speaker and/or LEDs for visual feedback. After adjustment, when an optimum energy transfer condition is established, causing Vₛ to decrease below the predetermined threshold level, the alignment indicator 706 is turned off.

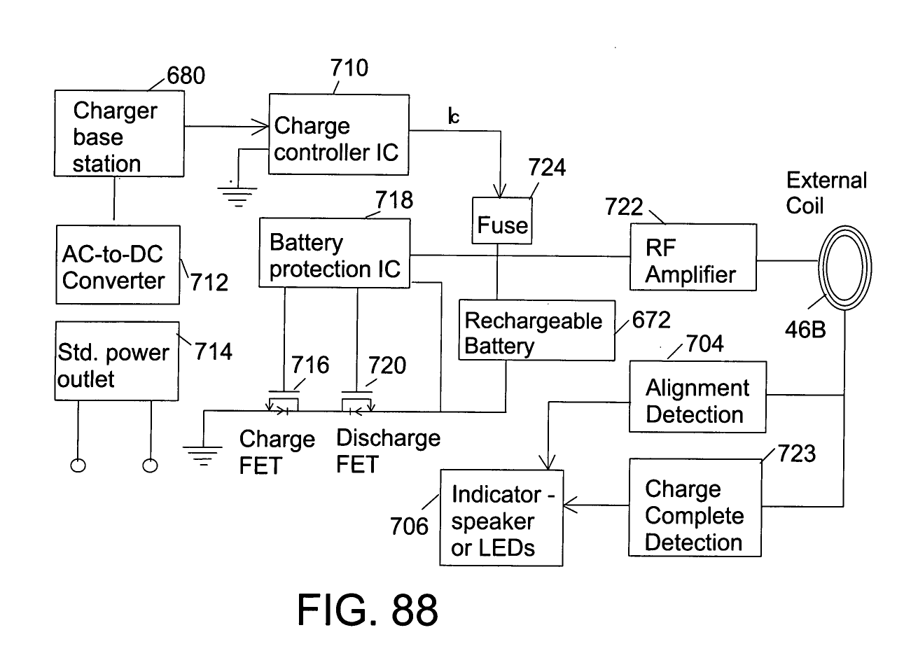

The elements of the external recharger are shown as a block diagram in conjunction with FIG. 88. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG 391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 88, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

In summary, the method of the current disclosure for neuromodulation of cranial nerve such as the vagus nerve(s), to provide therapy for neurological and neuropsychiatric disorders, can be practiced with any of the several power sources disclosed including,

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a programmable implantable pulse generator;

e) a combination implantable device comprising both a stimulus-receiver and a programmable IPG; and

f) an IPG comprising a rechargeable battery.

Neuromodulation of vagus nerve(s) with any of these systems is considered within the scope of this invention.

In one embodiment, the external stimulator and/or the programmer has a telecommunications module, as described in a co-pending application, and summarized here for reader convenience. The telecommunications module has two-way communications capabilities.

### Remote Telemetry

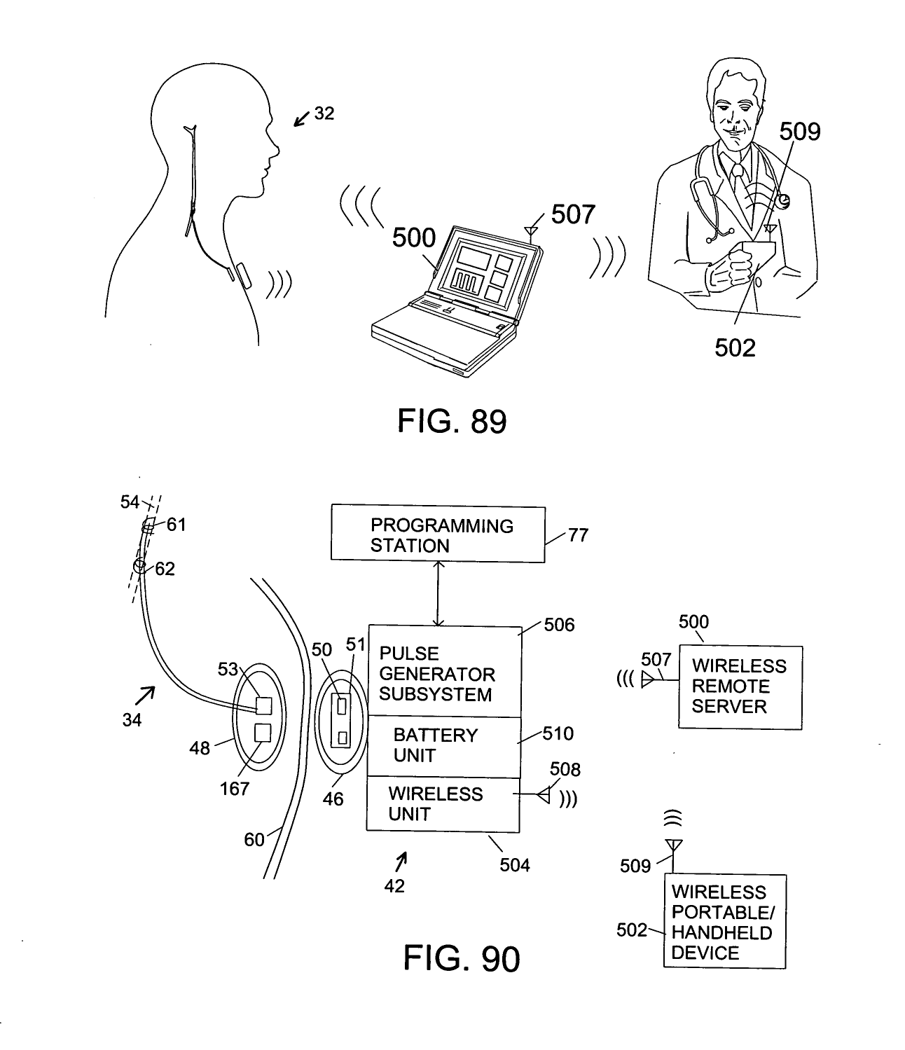

FIGS. 89 and 90 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

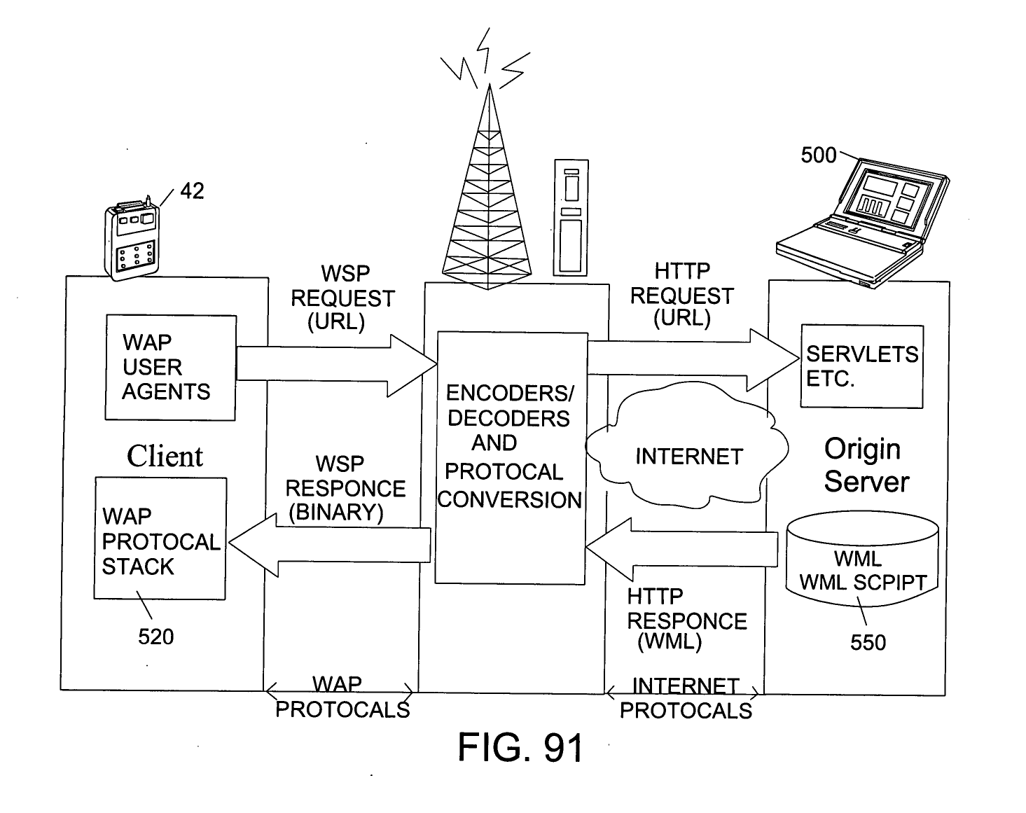

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 91. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 91, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

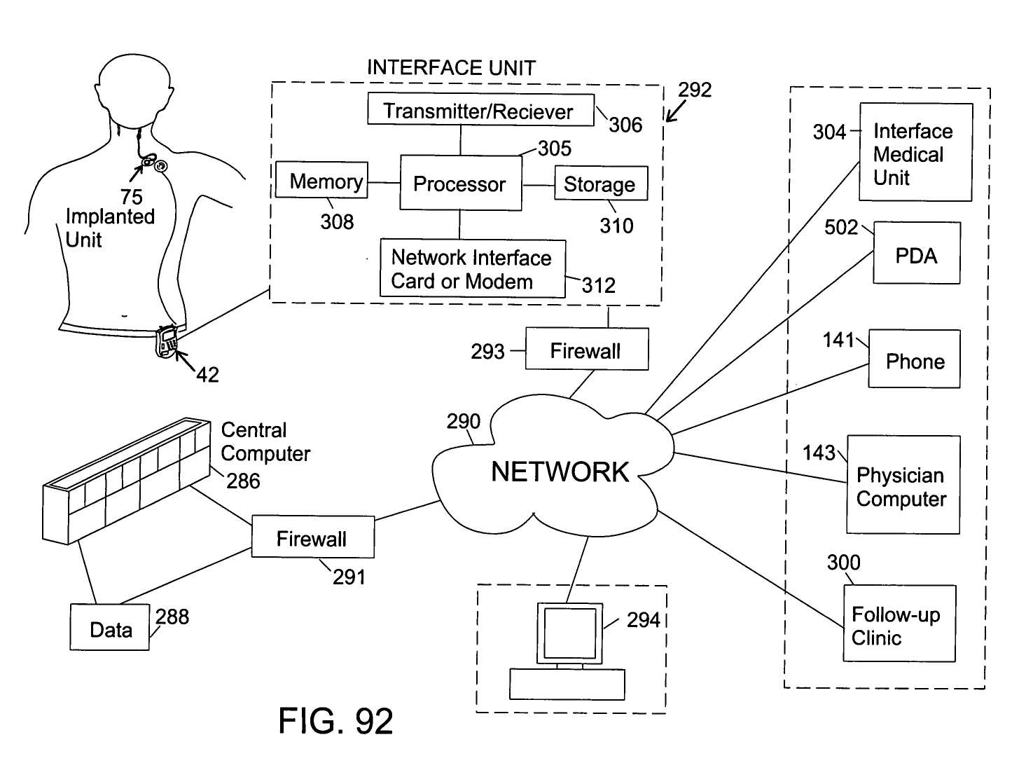

Shown in conjunction with FIG. 92, in one embodiment, the external stimulator 42 and/or the programmer 85 or patient controller 470 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

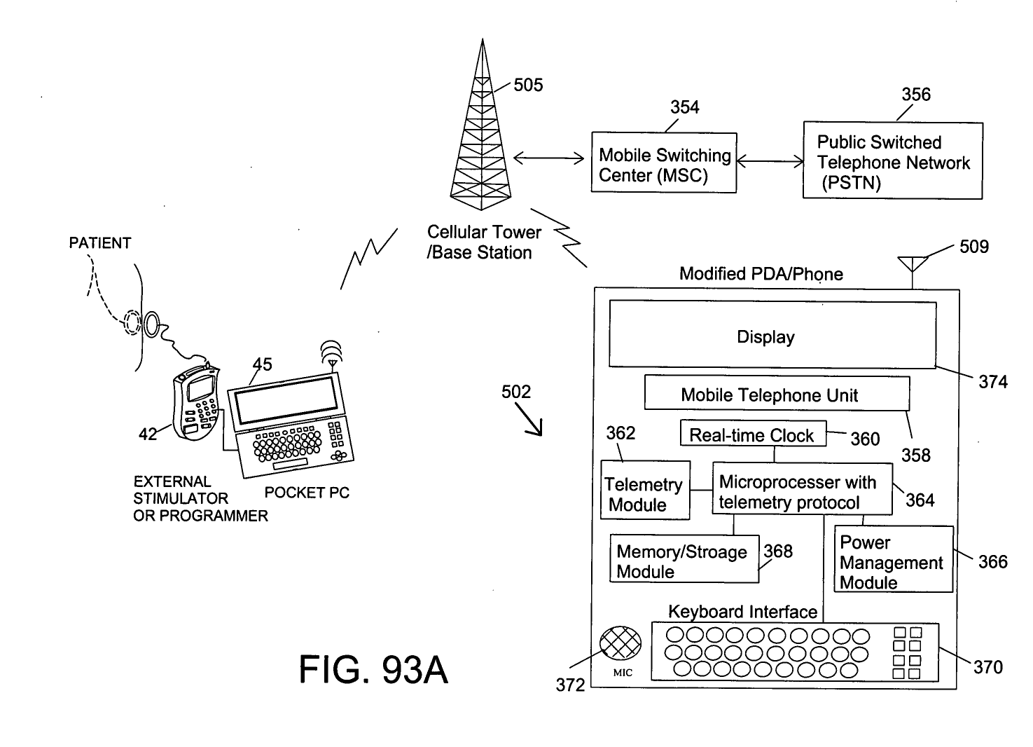

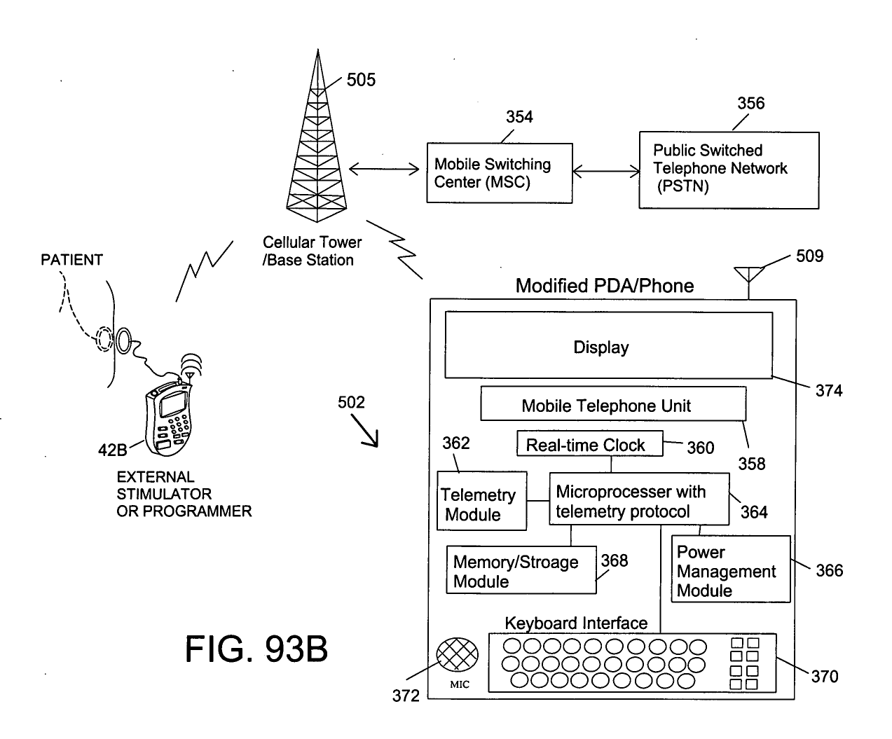

Shown in conjunction with FIGS. 93A and 93B the physician's remote communication's module is a Modified PDA/Phone 502 in this embodiment. The Modified PDA/Phone 502 is a microprocessor based device as shown in a simplified block diagram in FIGS. 79A and 79B. The PDA/Phone 502 is configured to accept PCM/CIA cards specially configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 502, is considered within the scope of the invention. Further, it will be evident to one of ordinary skill in the art that as future 4G systems, which will include new technologies such as improved modulation and smart antennas, can be easily incorporated into the system and method of current invention, and are also considered within the scope of the invention.

### Networking Combined with Wireless Telemetry

As was previously mentioned, in one aspect the communication of the implanted pulse generator (IPG) 391T and an external device may be via wireless telemetry utilizing the MICS band. With the availability of wireless telemetry, the IPG 391T is able to communicate with health care professional in real time or near real time.

This communication may be facilitated by means of a repeater—a device for receiving electronic communication signals and delivering corresponding amplified ones. In embodiments where the IPG is utilizing sensing, it would be desirable to have a base station 750 which may additionally process data received via such electronic communication signals prior to forwarding the data to a remote location. Data can be forwarded from the base station 750 via telephone land line, wireless cell phone communication, or the Internet to a doctor or medical professional.

With a two-way RF link, doctors can remotely monitor the patients or devices and wirelessly adjust the performance of the implanted device. Implanted devices may be programmed via the base station 750 and its associated data link(s). Such programming could replace the magnetic wand programming of the current systems which typically must be performed in a health care facility or doctor's office.

An implantable device such as the IPG 391T of the current disclosure may be paired with a base station or repeater 750 and linked by MICS transceivers. In such a system, data from the IPG 391T may be downloaded to the base station for data processing and analysis using higher performance data processing equipment (which typically has higher power consumption than lower performance processors). Moreover, the base station may provide a communication interface to telecommunications networks such as the Public Switched Telephone Network (PSTN), computer networks including the Internet, and radio-based systems including cellular telephone networks, satellite phone systems and paging systems.

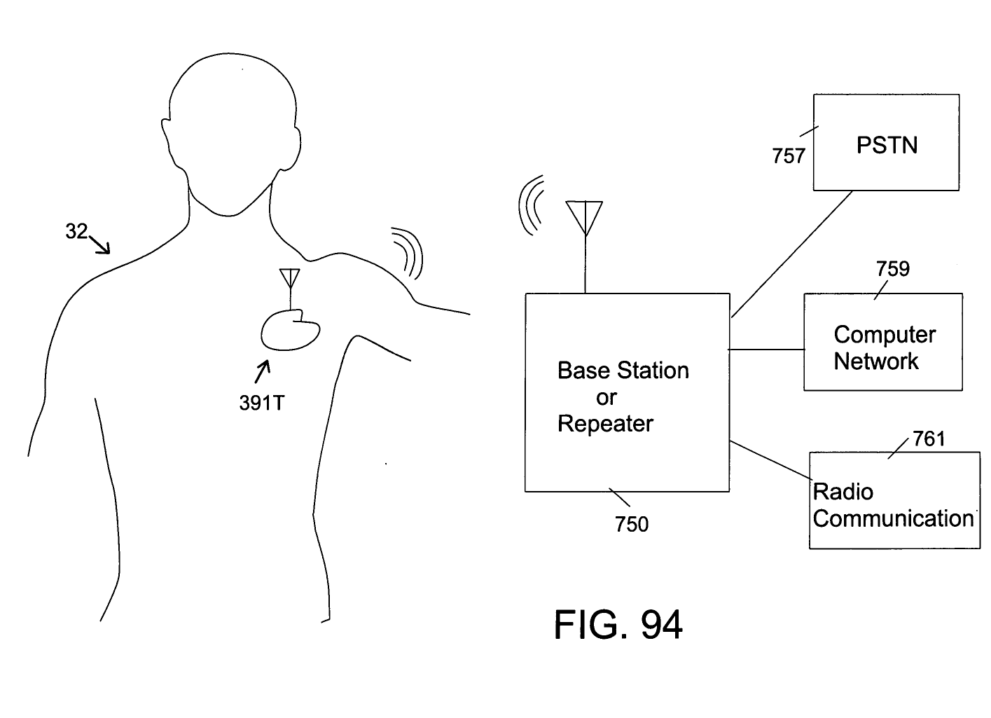

Shown in conjunction with FIG. 94, IPG 391T includes a short-range radio transceiver which utilizes the Medical Implant Communication Service. A corresponding transceiver in base station 750 receives data from device 391 T, processes and/or stores the data and sends it to a remote location using one or more of the Public Switched Telephone Network (PSTN) 757, computer network 759, and radio communications system 761. Computer network 759 may be a local area network (LAN), wide area network (WAN), an intranet or internet. Radio communications system 761 may, in certain embodiments, be a cellular telephone system, the PCS system, a satellite phone system, a pager system or a two-way radio link.

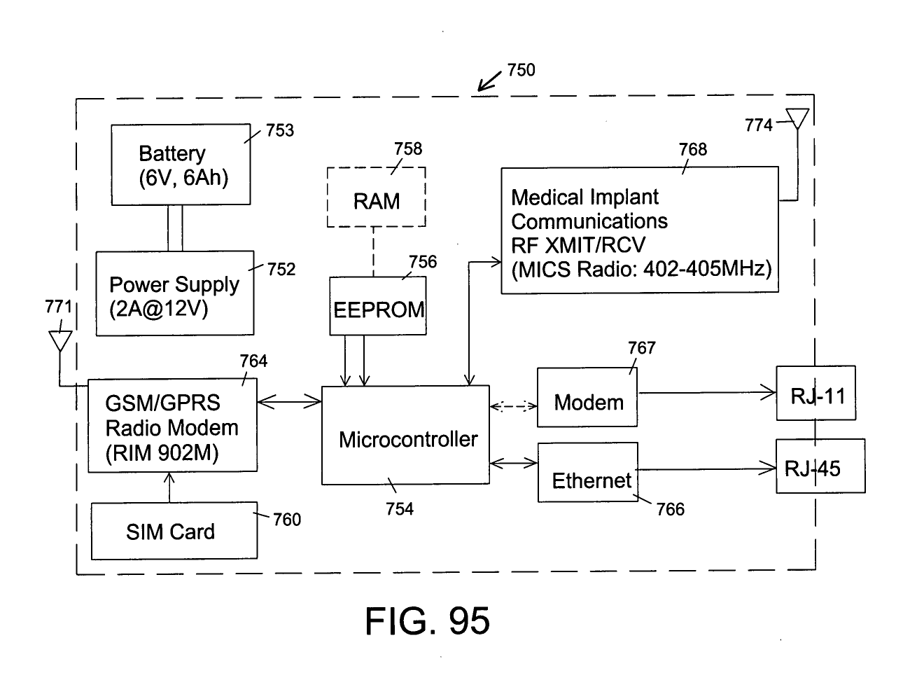

FIG. 95 is a block diagram of an exemplary base station or repeater 750. A power supply 752 may rectify and convert AC line voltage to DC at the voltage level(s) required by the various sybsystems within the base station 750. In some embodiments, power supply 752 may include an uninterruptible power supply (UPS) or battery 753 for operation during utility power interruptions or to permit brief operation of the base station at locations without external power. Power supply 752 may use an external wall transformer to deliver 9 or 12 volts DC to the system. An internal DC-DC converter may be used to step the voltage down to 3.3V (digital supply) or other appropriate value and 5V (analog supply & radio(s) supply). An internal DC-DC converter may help to reduce noise (60 Hz line noise, etc). This would help the SNR (Signal to Noise Ratio) of both the wireless data radio modem, and the medical band radio—improving the range and efficiency of both.

Processor (microcontroller) 754 may be a microprocessor or similar programmed system for implementing the methods of the system and controlling the various subsystems comprising base station 750. As noted above, one particularly significant advantage of base station 750 is its ability to use a powerful processor 754 whose electrical power consumption would be prohibitive for use within a battery-operated, energy sensitive implanted device such as IPG 391T of the current disclosure. Microcontroller 754 may be an 8 bit, 16 bit, 32 bit, or even 64 bit microcontroller.

Attached EEPROM 756 may be used for code/firmware storage, or additionally used as a temporary storage location for data in the event that a network connection is not immediately available. Attached RAM 758 may be used for code execution/scratchpad, or additionally used as a data buffer during transmit or receive.

Certain embodiments of base station 750 may optionally include display or alarms (not shown) for displaying operational and/or alerting the patient or caregiver of parameters which exceed defined limits.

For short-range communication with IPG 391T, base station or repeater 750 may include MICS transceiver 768 and antenna 774. Electrically small antennas are generally considered to be those with major dimension less than 0.05 lambda, or in the MICS band, 37 mm. In some embodiments the corresponding antenna with which the base station or repeater 750 communicates may be folded within the case of IPG. In other embodiments, the antenna may be outside of the IPG and encased in epoxy resin or other bio-compatible dielectric material, as was previously shown in FIGS. 65A and 65B. In this way, the usually metal case will not significantly impede RF transmission to and from the antenna of IPG.

The MICS transceiver 768 enables communication with the implantable pulse generator (IPG) 391T. It may operate on a different frequency than the GSM bands to avoid interference with radio modem 764. Interface 768 provides high-speed wireless data communication with the implanted pulse generator 391T, within approximately 6 feet. A stationary base station 750 could be place near a bed or other location where the patient could be close enough for data transmission. Similarly, a portable device could be worn by the patient, including wearable computer as discussed later.

For data communication with remote locations, such as doctor's offices, base station 750 may include network interface 766. One example of network interface 766 is an Ethernet Network Interface Card (NIC). Ethernet can be used as an alternative connection to the Internet for uploading patient data, in the event that wireless data service is not available or is expensive. Ethernet interface 766 can also be used for remote management and uploading/upgrading system firmware. An optional modem 767 for data transmission using the public switched telephone network 757 may also be incorporated.

An alternative data communication interface for base station 750 is radio modem 764. In certain embodiments, radio modem 764 may be a cellular telephone with a modem, or may be operated similarly thereto. Radio modem 764 may couple to an antenna 771 which, in some embodiments, may be external to or remote from the main housing of base station 750. The specific design of the antenna depends on the particular band used, but in any event could conform with GSM 900, 850, 1900, or 1880 standard. Use of a dedicated GSM/GPRS radio modem 764 can reduce system complexity, as this would require only a power supply and a data connection to the system. This reduces overall system complexity.

Data may be transmitted to the Internet using GPRS (General Packet Radio Service) over the GSM band. Alternatively, instead of a self-contained radio modem, a quad-band (or tri-band) GSM/GPRS RF (Radio Frequency) transceiver can be used. However only the radio and associated components are included, so further additional hardware might be required for baseband processing, etc. This can connect to and switch between a greater number of GSM networks, allowing for greater coverage area.

Alternatively, instead of GPRS/GSM, a different cellular data service, such as X.25 or Cellular Digital Packet Data (CDPD) can be used. One preferred embodiment uses a RIM 902M Radio Modem operating in the GSM 900 band. For example, RIM's proprietary Radio Access Protocol could be used to communicate with the modem 764 in this example. Alternatively, if a self-contained radio modem from another manufacturer is used, a different protocol, such as RS-232, may be used.

In other embodiments, if a custom GSM/GPRS RF solution is used instead of a self-contained radio modem, a custom interface could be defined, for example using memory-mapped I/O, or simply the GPIO (General Purpose I/O) pins on the controller to communicate with and control the radio.

SIM Card 760 is a Subscriber Identity Module that identifies a particular user of a GSM network. SIM card 760 could be keyed to a Patient ID, for example, for billing purposes, Patient ID could be encrypted and sent separately along with patient data.

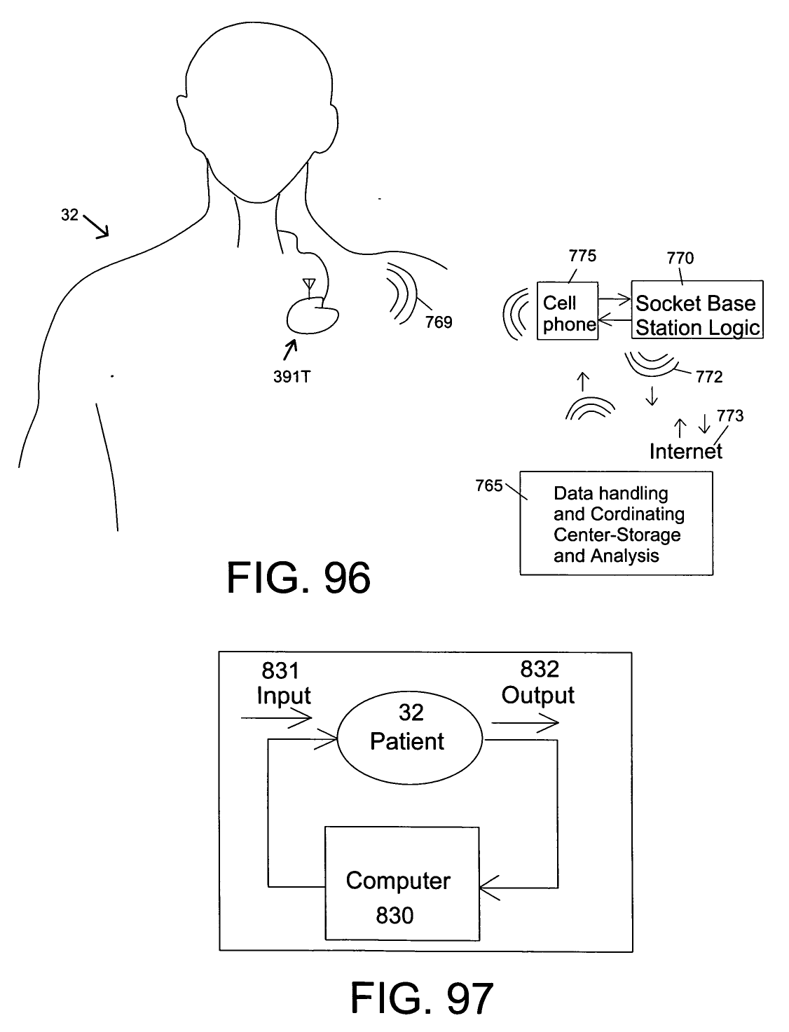

FIG. 96 illustrates another embodiment of networking using wireless telemetry 769. In this embodiment, communication occurs with a cell phone 775 which acts as a repeater/base station programmed with appropriate logic 770 to perform the function of the base station as discussed above. Such logic 770 can appear within the phone 775 itself, or in a traditional phone socket or cradle. Cell phone 775 communicates via an RF interface with the implant device 391T, and further communicates via an RF telephone link 772 to, for example, the Internet 773, which can comprise one network intervening between the phone 775 and a coordination center 765, where data is stored and/or analyzed. One skilled in the art will realize that other communication networks in addition to the Internet would logically be used, but are not shown. If needed, alerts can be sent to the patient via the Internet 773 either to the cell phone 775, or through wireless cell phone communication to cell phone 775. Further communication of the alert to the patient can then be communicated through radio frequency link 769 to the IPG 391T.

### Wearable Computer

As previously mentioned, the implanted pulse generator (IPG) 391T communicates wirelessly with an external programmer or an external computer. In one aspect of this disclosure, for patient convenience the external computer may be a wearable computer, in which the system components are distributed on a patient's body (which is currently available or to be developed). Advantageously, the wearable computer system is minimally obstrusive to the movements and actions of the user 32, and in the future wearables will likely be almost invisible, integrating seamlessly with everyday clothing and accessories.

A wearable computer 830 is a computer that is subsumed into the personal space of the user (patient 32), controlled by the user, and has both operation and interactional constancy, i.e. is always on and always accessible. Most notably, it is a device that is always with the user, and into which the user can always enter commands and execute a set of such entered commands, and in which the user can do so while walking around or doing other activities. Generally a salient aspect of computer whether wearable or not, is their reconfigurability and their generality, e.g. that their function can be made to vary widely, depending on the instructions provided for program execution. With the wearable computer, this is no exception, e.g. the wearable computer is more than just a wristwatch or regular eyeglasses: it has the full functionality of a computer system but in addition to being a fully featured computer, it is also inextricably intertwined with the wearer.

Generally, the assumption of wearable computing is that the user (patient 32) will be doing something else at the same time as the computer is being used. The signal flow between patient 32 and the computer 830 will serve to augment some function. The signal flow between patient 32 and the computer 830 is depicted in FIG. 97.

Wearable computers are currently available from several vendors such as, Symbol Technologies, Circus Systems Wearable, Charmed Technology, EMJ Embedded Systems, Xybernaut, and Adastra among some of the venders.

For example, one wearable computer currently available from Symbol Technologies uses Windows CE 5.0. It runs the 520-MHz Intel Xscale PXA270 processor, with 128 MB of RAM and 68 MB of flash memory. Of course the capacity will improve significantly over time, and the wearable computer 830 will be able to communicate with the implanted pulse generator (IPG) 391T, and with other computers that are situated remote to the patient.

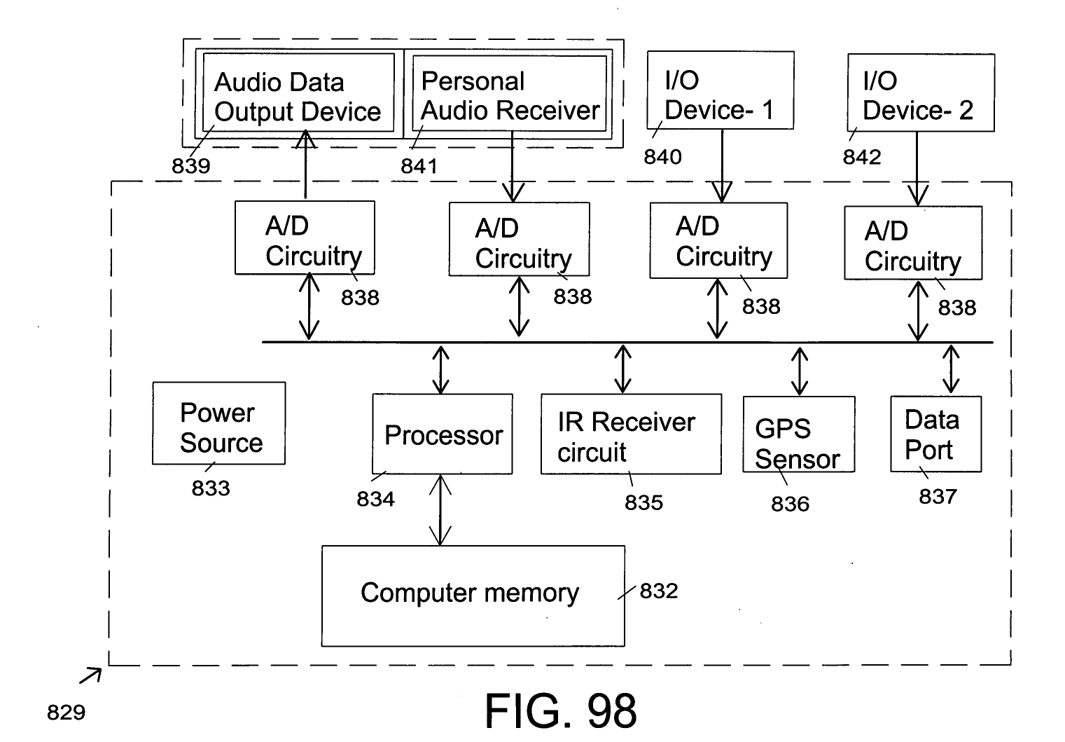

A general block diagram of a wearable computer 830 is shown in conjunction with FIG. 98. A processor 834 is connected to a computer memory 832 inside the computer unit 829. A power source 833, such as a battery, may be housed within a computer unit 829 for supplying power to all the circuitry in the system. The computer may optionally comprise a personal microphone 839, in such a case the personal microphone receives audio signals from the user (patient 32) and sends electrical signals, such as analog signals, to the computer unit 829. The computer unit 829 includes conventional analog-digital circuitry 838 that digitizes the analog signal from the personal microphone 839. The computer memory 832 includes a voice recognition engine that receives the digitized signals from the analog-digital circuitry 838 and interprets the proper commands to be executed by the processor 834. Similar analog-digital circuitry 839 may be connected to personal audio receiver 841, the environmental microphone (not shown), and other input/output (I/O) devices 840, 842.

While the data input directly from the user 32 to the wearable computer system 829 consists of audio data, the wearable computer 830 may automatically input data from other sources that do not employ a user interface. A conventional GPS sensor 836 to input the location of the user 32 may be enclosed inside the computer unit 829 of the wearable computer 830 and connected to the processor 834.

A data port 837 is used to upload saved data from the computer unit 829 directly to a remote computer (not shown) or to download information, such as software updates, from the remote computer to the computer unit 829. The data port 837 may use a conventional connection to the remote computer, such as a USB or IR port, or a wireless network connection. In one embodiment, the data port 837 of the computer unit 829 may be connected to a wireless radio frequency (RF) transmitter (for example, a cellular telephone), for transmissions to or from another person or remote computer. The data port 837, the GPS sensor 836, and the IR receiver circuit 835 are all examples of sources that may be used by the wearable computer system 830 to input information without employing a user interface, and thus enabling the wearable computer system 830 to be less noticeable on the user 32.

In one embodiment a personal digital assistant (PDA), or hand-held computer, may be integrated with the computer unit 829, or serve as the computer unit 829. As such, the PDA provides a display for the user when hands-free operation is not needed.

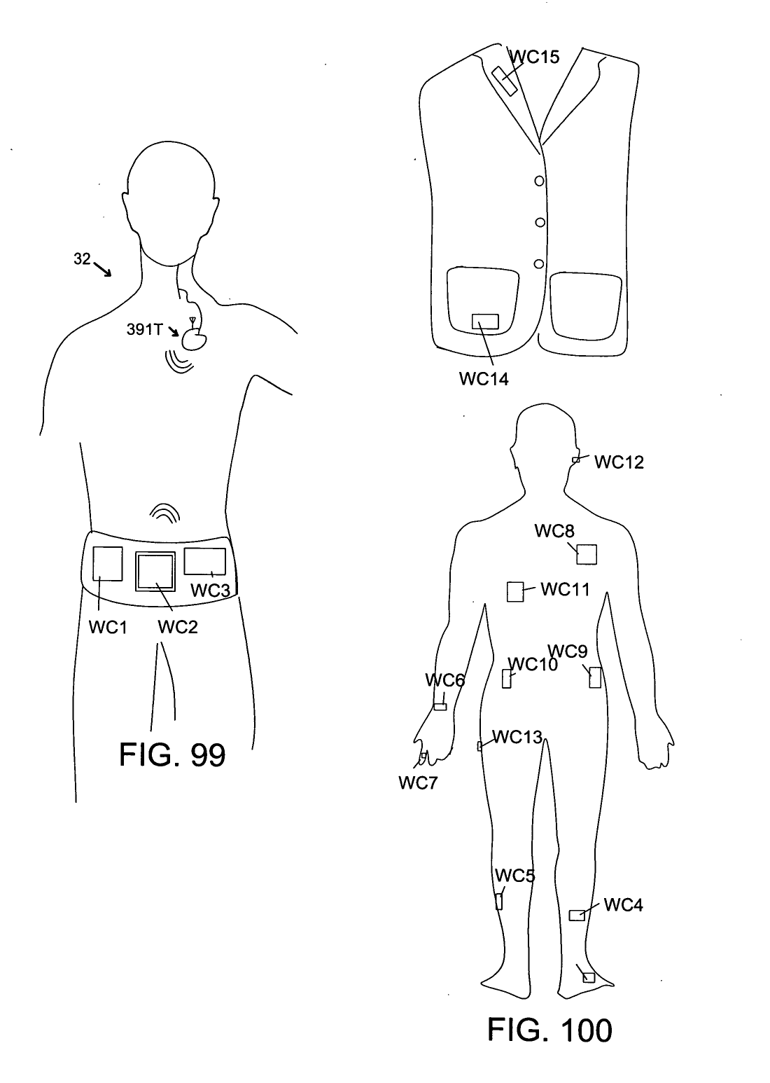

Additional measures may be taken to make the wearable computer system 830 even more unintrusive for the user and people who interact with the user. For example FIG. 99 show the computer unit 829 attached to the belt 844 on user 32, but the computer unit 15 may alternatively be carried in a pocket of the user's clothing, depending on the size of the computer's unit 829. Further examples of different embodiments of wearable computers are shown in conjunction with FIG. 100, where wearable computer such as WC1, WC2, . . . WC14 are incorporated into the clothing and placement examples on a patient are depicted.

In another aspect of the invention, the wearable computer system 830 uses natural voice commands from the user 32. The predetermined voice commands, whether natural or explicit, may be customized by the user through a set-up procedure.

In a further embodiment of the invention, the data port 837 may use a conventional wireless connection to upload and down load information between a remote computer and the computer unit 829.

### Device Identification for Follow-Up

In one aspect, for follow-up convenience and identification the implanted pulse generator (IPG) 391 or the patient 32 may also be equipped with a radio frequency identification (RFID) tags 850. Alternatively or additionally, the patient 32 may be provided with a card, bracelet, or a smart card. Advantageously, device and/or patient information may be incorporated in the smart card which can be updated periodically. The device information can include, for example, model number, serial number, and any statement regarding the implanted system such as single lead, dual leads, type of lead etc. or like information. The patient information, for example, can include patient's name, physician's name, underlying condition, brief history as desired.

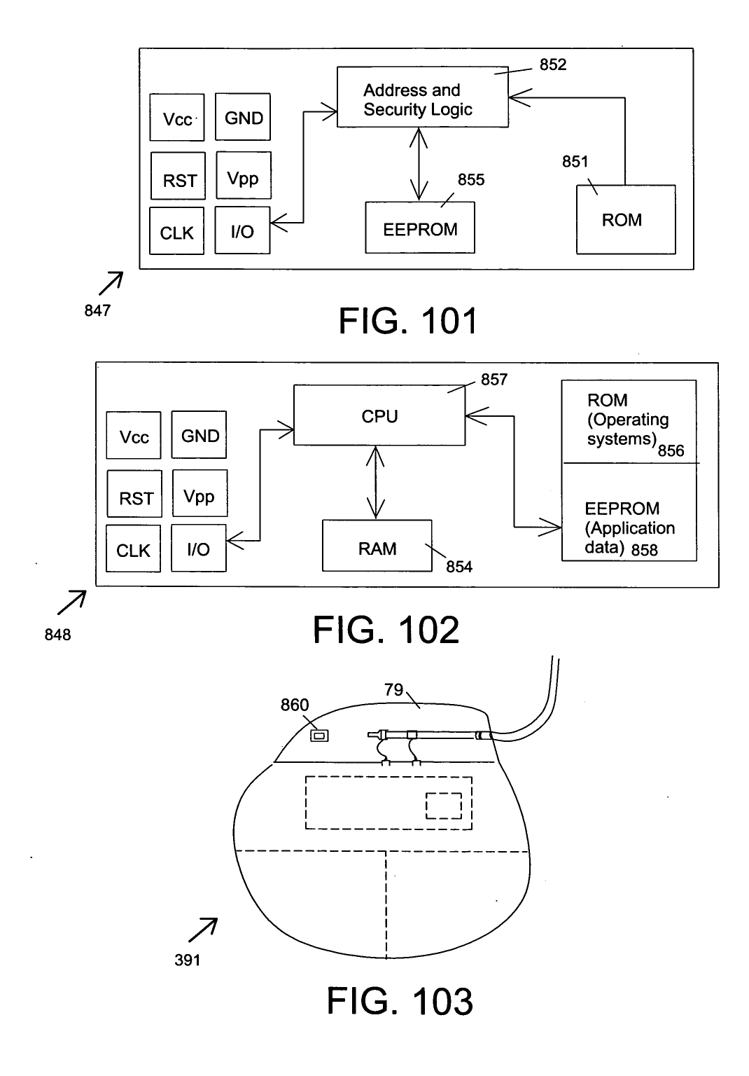

A smart card is an electronic data storage system, possibly with additional computing capacity (microprocessor card), which for convenience is incorporated into a plastic card the size of a credit card. The smart card is supplied with energy and a clock pulse from the reader via contact surfaces. Data transfer between the reader and the card takes place using a bidirectional serial interface (I/O port). In the method and system of this disclosure, two basic types of smart cards may be used which are a memory card 847 or a microprocessor card 848, which are differentiated based upon their functionality. Shown in conjunction with FIG. 101, is block diagram of a memory card 847. In memory cards 847 the memory 851 usually an EEPROM 855 is accessed using a sequential logic (state machine). In one aspect simple security algorithms, e.g. stream ciphering may be incorporated. The functionality of the memory card 847 is adapted and optimized for the current application.

In one aspect, a microprocessor card 848 may be provided to the patient. A typical architecture of a microprocessor card 848 is shown in conjunction with FIG. 102. As depicted in FIG. 102, the microprocessor card contains a microprocessor 857, which is connected to a segmented memory (ROM 856, RAM 854, and EEPROM 858 segments).

The mask programmed ROM 856 incorporates an operating system (higher program code) for the microprocessor 857 and is inserted during chip manufacture. The contents of the ROM 856 are determined during manufacturing, are identical for all microchips from the same production batch, and cannot be overwritten.

The chip's EEPROM 858 contains application data and application-related program code. Reading from or writing to this memory area is controlled by the operating system. The RAM 854 is the microprocessor's temporary working memory. Data stored in the RAM 854 are lost when the supply voltage is disconnected.

Microprocessor cards are very flexible. In modern smart card systems it is also possible to integrate different applications in a single card (multi-application). The application-specific parts of the program are not loaded in the EEPROM until after manufacture and can be initiated via the operating system. Microprocessor cards 848 are also used in many security sensitive applications, which brings the price to a reasonable level for the current application. The option of programming the microprocessor cards 848 also facilitates the use for the current application.

### RFID Tag for Device Identification

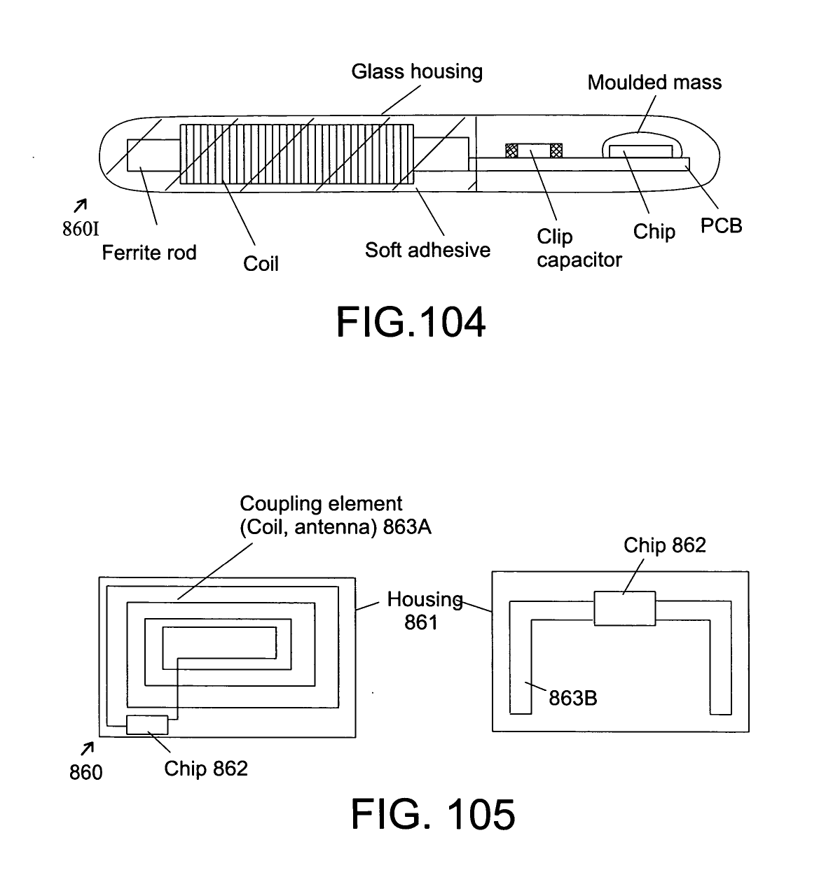

For follow-up device identification and convenience in addition to a card or bracelet, or a smart card, a Radio Frequency Identification (RFID) tag may be placed in the implanted device or the patient 32. In one embodiment (shown in conjunction with FIG. 103), the RFID tag 860 may be placed in the header portion 79 of the IPG 391, which is typically made of silicone or like material. In another embodiment, a separate stand alone RFID tag may be injected or implanted anywhere in the body. An example of a prior art injectable RFID tags is shown in FIG. 104. A stand-alone RFID tag may be encased in glass or may have ceramic housing. Stand alone RFID tag such as depicted in FIG. 104 is injected into the body, as is known in the art. Advantageously, these RFID tags are passive devices and do not require a battery.

In one aspect, when a patient 32 with the implanted device appears at any health care facility, a reader (or interrogator) scans the patient 32 and is flashed back with a unique code and a web address of the manufacturer. The health care professional then navigates to the web site and enters the unique code to get all the relevant information about the device and any special instructions about programming the device, or like information.

The detailed technology of RFID tags is well known in the art, and the reader is also referred to RFID Handbook-Fundamental's and Application in Contactless Smart Cards and Identification (Second Edition), by Klaus Finkenzeller—Wiley Press, the relevant contents of which are incorporated herein in its entirety by reference.

As is well known in the art the RFID system comprises an RFID tag, which in the current application is implanted in the body. An external reader (also known as interrogator or hand held scanner) wirelessly communicates with the transponder. In one embodiment 13.56 MHz frequency range may be used (the range of 13.553-13.567 MHz). Other frequency ranges such as 6.78 MHz, 27.125 MHz, 40.680 MHz or even other frequencies may also be used in this application.

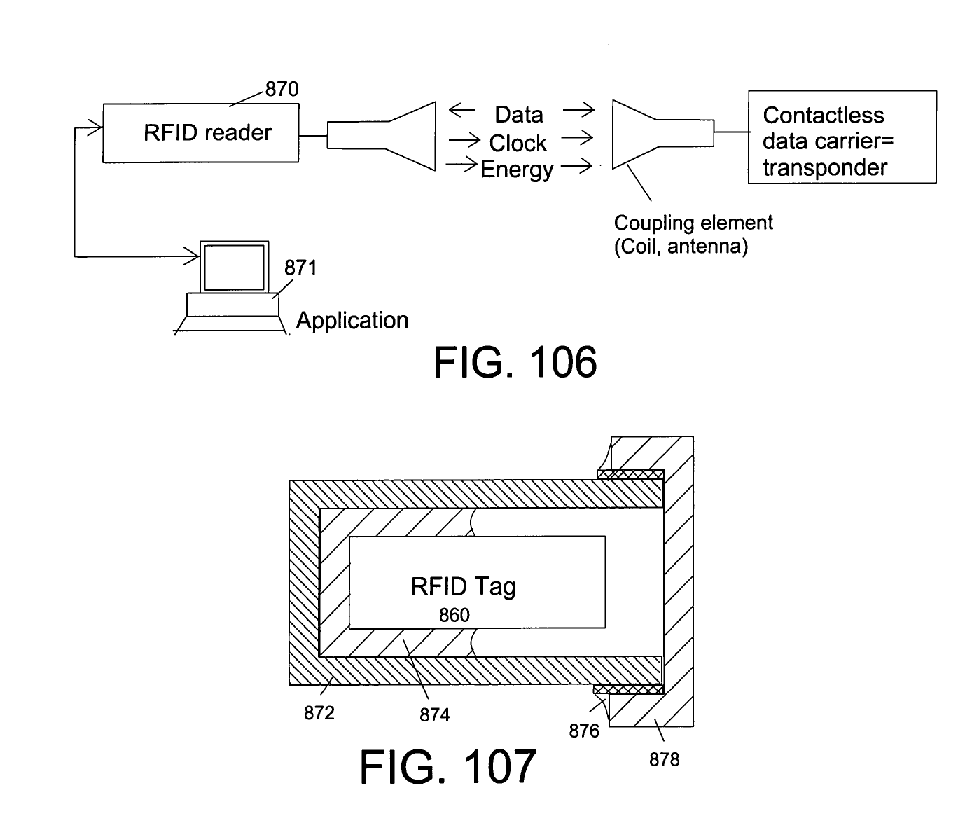

As shown in conjunction with FIG. 105, the RFID tag 860 comprises a substrate 861, an RFID chip 862, and an antenna or coil 863 for both receiving electromagnetic energy to power the RFID chip 862 and for retransmitting a digital pulse. For operation of the RFID system, shown in conjunction with FIG. 106, the interrogator (RFID reader) 870 with associated antenna discharges electromagnetic energy to the antenna 863 of the RFID tag 860, which powers up the RFID chip 862 and allows it to produce the electromagnetic return signal. The electromagnetic return signal is detected by the interrogator 870 and presented as a digital code sequence. In addition, as is shown in FIG. 106 the reader may be fitted with an additional interface (RS 232, RS 485, etc.) to enable them to forward the data received to another system including a PC system 871.

The RFID tag 860 may be read-only (RO) or read/write (RW). With an RW RFID tag 860, a physician may use an external programmer or interrogator 20 to write additional patient information to the RFID tag 860. The interrogator 870 may comprise programmer or programmer/reader, which would permit direct display of all of the information contained on the RFID tag 860.

One skilled in the art will appreciate that the injectable version of RFID tags will need to be enclosed in a biocompatible and hermatically sealed containers. Since hermaticity is important for the current application, the RFID tag 860 is preferably encased in a ceramic housing 872 such that the components of the RFID are hermetically sealed. This is shown in conjunction with FIG. 107. The RFID tag 860 is placed in a ceramic housing 872 where it is surrounded by a ceramic encapsulant 874. The ceramic housing 872 is capped with a ceramic or metallic cap 878. A gold brazed joint 876 for metallergical hermetic connection is typically used for binding the typically metallic end cap 878 to the ceramic housing 872.

The hermatically sealed housing is preferably made of ceramic material. Advantageously, a ceramic housing allows electromagnetic fields to freely pass to and from the RFID tag 860. The ceramic housing 872 is generally made by taking alumina ceramic powders which has been formulated with binder system and making them into the desired shape. Further, before sealing or capping the ceramic cell dessicants such as anhydrous magnesium and calcium sulfate may be added to absorb moisture. These dessicants have a very strong affinity for water.

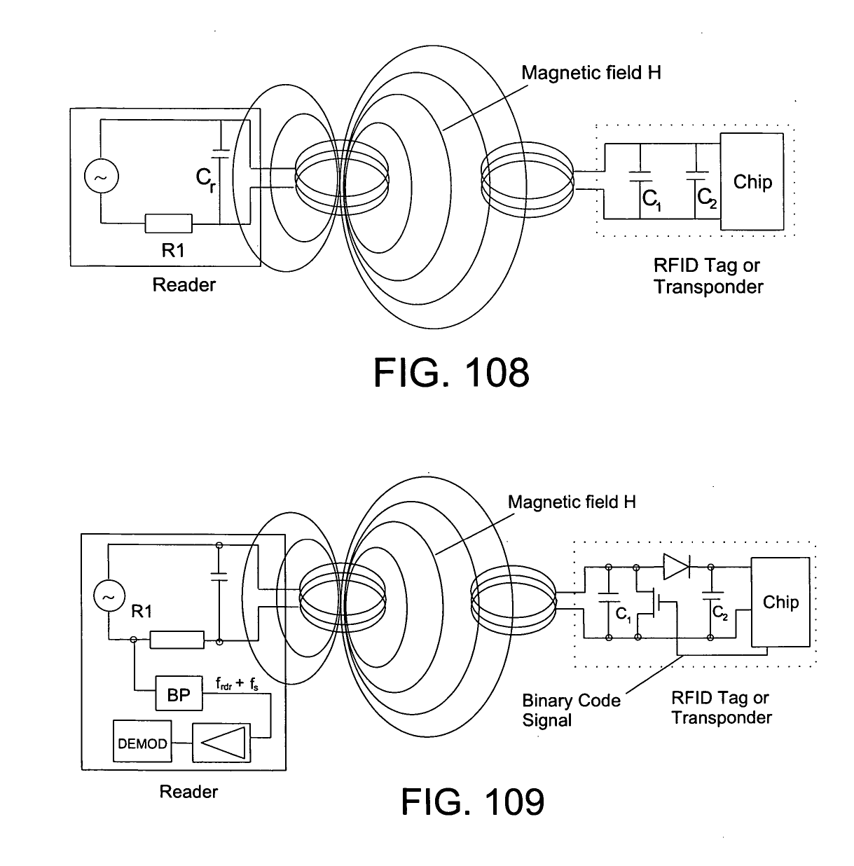

The operation of the RFID device is described below in conjunction with FIGS. 108 and 109. An inductively coupled transponder comprises an electronic data-carrying device, usually a single microchip, and a coil that functions as an antenna.

Inductively coupled transponders are operated passively. This means that all the energy needed for the operation of the microchip has to be provided by the reader. This is shown in conjunction with FIG. 108. For this purpose, the reader's antenna coil generates a strong, high frequency electromagnetic field, which penetrates the cross-section of the coil area and the area around the coil. Because the wavelength of the frequency range used (e.g. 13.56 MHz) is several times greater than the distance between the reader's antenna and the transponder, the electromagnetic field may be treated as a simple magnetic alternating field with regard to the distance between transponder and antenna.

A small part of the emitted field penetrates the antenna coil of the transponder, which is some distance away from the coil of the reader. A voltage Uᵢ is generated in the transponder's antenna coil by inductance. This voltage is rectified and serves as the power supply for the data-carrying device (microchip). A capacitor Cᵣ is connected in parallel with the reader's antenna coil, the capacitance of this capacitor being selected such that it works with the coil inductance of the antenna coil to form a parallel resonant circuit with a resonant frequency that corresponds with the transmission frequency of the reader. Very high currents are generated in the antenna coil of the reader by resonance step-up in the parallel resonant circuit, which can be used to generate the required field strengths for the operation of the remote transponder.

The antenna coil of the transponder and the capacitor C1 form a resonant circuit tuned to the transmission frequency of the reader. The voltage U at the transponder coil reaches a maximum due to resonance step-up in the parallel resonant circuit.

The layout of the two coils can also be interpreted as a transformer (transformer coupling), in which case there is only a very weak coupling between the two windings . The efficiency of power transfer between the antenna coil of the reader or interrogator and the transponder (or RFID) tag is proportional to the operating frequency f, the number of windings n, the area A enclosed by the transponder coil, the angle of the two coils relative to each other and the distance between the two coils.

As frequency f increases, the required coil inductance of the transponder coil, and thus the number of windings n decreases (for 13.56 MHz: typically 3-10 windings). Because the voltage induced in the transponder is still proportional to frequency f, the reduced number of windings barely affects the efficiency of power transfer at higher frequencies.

If a resonant transponder (i.e. a transponder with a self-resonant frequency corresponding with the transmission frequency of the reader) is placed within the magnetic alternating field of the reader's antenna, the transponder draws energy from the magnetic field. The resulting feedback of the transponder on the reader's antenna can be represented as transformed impedance Z_{T} in the antenna coil of the reader. Switching a load resistor on and off at the transponder's antenna therefore brings about a change in the impedance Z_{T}, and thus voltage changes at the reader's antenna. This has the effect of an amplitude modulation of the voltage U_{L} at the reader's antenna coil by the remote transponder. If the timing with which the load resistor is switched on and off is controlled by data, this data can be transferred from the transponder to the reader. This type of data transfer is load modulation.

To reclaim the data at the reader, the voltage tapped at the reader's antenna is rectified. This represents the demodulation of an amplitude modulated signal.

Shown in conjunction with FIG. 109, due to the weak coupling between the reader antenna and the transponder antenna, the voltage fluctuations at the antenna of the reader that represent the useful signal are smaller by orders of magnitude than the output voltage of the reader.

In practice, for a 13.56 MHz system, given an antenna voltage of approximately 100 V (voltage step-up by resonance) a useful signal of around 10 mV can be expected (=80 dB signal/noise ratio). Because detecting this slight voltage change requires highly complicated circuitry, the modulation sidebands created by the amplitude modulation of the antenna voltage are utilized (FIG. 109).

If the additional load resistor in the transponder is switched on and off at a very high elementary frequency f_{S}, then two spectral lines are created at a sistance of +/−f_{S} around the transmission frequency of the reader f_{READER}, and these can be easily detected (however f_{S} must be less than f_{READER}). In the terminology of radio technology the new elementary frequency is called a subcarrier. Data transfer is by ASK, FSK or PSK modulation of the subcarrier in time with the data flow. This represents an amplitude modulation of the subcarrier.

Load modulation with a subcarrier creates two modulation sidebands at the reader's antenna at the distance of the subcarrier frequency around the operating frequency f_{READER}. These modulation sidebands can be separated from the significantly stronger signal of the reader by bandpass (BP) filtering on one of the two frequencies f_{READER}+/−f_{S}. Once it has been amplified, the subcarrier signal is now very simple to demodulate.

Because of the large bandwidth required for the transmission of a subcarrier, this procedure can only be used in the ISM frequency ranges for which this is permitted, 6.78 MHz, 13.56 MHz and 27.125 MHz.

Although various embodiments have been described in detail for purposes of illustration, various modifications may be made without departing from the scope and spirit of the invention.

What is claimed is:

1. A method of providing complex electrical pulses to a vagus nerve(s) its branches or parts thereof of a patient for treating or alleviating the symptoms for at least one of depression, anxiety disorders, autism, epilepsy and involuntary movement disorders, neurogenic/psychogenic pain, obsessive compulsive disorders, compulsive eating disorders, bulimia, obesity, dementia including Alzheimer's disease, or migraines, comprising the steps of:
providing a microprocessor based implantable pulse generator (IPG) capable of providing complex electrical pulses, and comprising at least one predetermined/pre-packaged program(s), electrical circuitry, and rechargeable or non-rechargeable battery;
providing an implanted lead(s) in electrical contact with said implanted pulse generator, wherein said implanted lead(s) comprise at least one electrode adapted to be in contact with said vagus nerve(s);
choosing a predetermined/pre-packaged program from at least one predetermined/pre-packaged program(s); and
activating said predetermined/pre-packaged program with a programmer using bi-directional telemetry, wherein said bidirectional telemetry utilizes magnetic inductive coupling or wireless telemetry within two meter range
whereby, complex electrical pulses are provided to said vagus nerve(s), its branches or parts thereof according to said at least one predetermined/pre-packaged program to provide therapy or alleviate symptoms for at least one of said disorders.

2. The method of claim 1, wherein said at least one predetermined/pre-packaged programs define unique combinations of variable electrical parameters.

3. The method of claim 1, wherein said predetermined/pre-packaged programs provides changes in regional cerebral blood flow (rCBF), and/or can alter neurochemicals in the brain, and/or can alter neural activity in the brain.

4. The method of claim 1, wherein said complex electrical pulses provided are in a range between 0 Hz and 5,000 Hz.

5. The method of claim 1, wherein said predetermined/pre-packaged program(s) are provided independently of synchronization or desynchronization patient's EEG.

6. The method of claim 1, wherein said predetermined/pre-packaged program(s) can be altered or modified.

7. The method of claim 1, wherein said predetermined/pre-packaged program(s) can be remotely interrogated and/or programmed over a network.

8. The method of claim 1, wherein said implantable pulse generator communicates wirelessly with a wearable computer on a patient, and the wearable computer is capable of being networked with remote computers.

9. The method of claim 1, wherein said implantable pulse generator further comprises a recharge coil which may be inside or outside a titanium case of said implantable pulse generator.

10. The method of claim 1, wherein patients implanted with said implantable pulse generator are provided with a smart card which comprises device and/or patient information, which can also be updated.

11. The method of claim 1, wherein said implantable pulse generator further comprises a radiofrequency identification tag (RFID) within a header of said implantable pulse generator for patient and/or device follow-up.

12. The method of claim 1, wherein a patient being implanted with said implantable pulse generator is also injected with a radiofrequency identification tag (RFID) which comprises device and/or patient information.

13. The method of claim 1, wherein said predetermined program(s) can be changed by a patient utilizing a patient programmer.

14. A method of providing neuromodulation with predetermined complex electrical pulses to a vagus nerve(s) to provide therapy for at least one of epilepsy, depression, anxiety disorders, neurogenic pain, compulsive eating disorders, obesity, dementia including Alzheimer's disease, and migraine, comprising the steps of:
providing a programmable implantable pulse generator capable of providing complex electrical pulses comprising microprocessor, electrical circuitry, memory, and power source;
providing an implantable lead in electrical contact with said implantable pulse generator, and at least one electrode adapted to be in contact with said vagus nerve(s);
providing an external programmer comprising circuitry that programs said implantable pulse generator using magnetic inductive coupling or wireless telemetry for bi-directional data exchange, wherein said external programmer being capable of network connection for remote communication using a wide area network;
programming said implanted pulse generator with said external programmer to deliver predetermined electrical pulses for providing therapy for at least of said disorders; and
remotely communicating with said external programmer for data exchange over a wide area network.

15. The method of claim 14, wherein said neuromodulation is performed independently of synchronization or desynchronization of patient's electroencephalogram (EEG).

16. The method of claim 14, wherein said electrical pulses are further provided alone or as adjunct therapy with at least one of drug therapy, transcranial magnetic stimulation (rTMS) therapy, or electroconvulsive therapy (ECT), in any combination or sequence to provide therapy or alleviate symptoms of depression.

17. The method of claim 14, wherein said electrical pulses are provided to further cause regional cerebral blood flow (rCBF) changes in at least one region of the brain, and/or alter neurochemicals.

18. A method of treating, controlling or alleviating the symptoms of neurological or neuropsychiatric disorders, comprising the steps of:
selecting a type of pulse generator system suitable for a patient for providing complex electrical pulses to a vagus nerve;
implanting said selected pulse generator system; and
applying a predetermined/pre-packaged program of complex electrical pulses to a vagus nerve(s) its branches or part thereof for altering regional cerebral blood flow (rCBF) in the patient to alleviate the symptoms of the neurological or neuropsychiatric disorder exhibited by the patient being treated.

19. The method of claim 18 wherein said neurological or neuropsychiatric disorders comprises depression, anxiety disorders, autism, epilepsy and involuntary movement disorders, neurogenic/psychogenic pain, obsessive compulsive disorders, compulsive eating disorders, bulimia, obesity, dementia including Alzheimer's disease, or migraines.

20. The method of claim 18, wherein said pulse generator system is one of; a combination implantable device wherein said implantable device comprises both a stimulus-receiver module and a programmable implanted pulse generator module (IPG), an implantable pulse generator (IPG) comprising a rechargeable battery, or a programmable implanted pulse generator (IPG).
